SEQUENCE LISTING (37 sequences)

SEQ ID NO: 1 - DNA, 3114 bp, Neisseria meningitidis
atggctgttattatatttgttaacggaattcgggctgtaaatggccttgttaaatcatctatcaatactgcaaacgcttttgctgaagaaggactggatgttcatttaattaattttgttggcaatattactggagcagagcatttataccccccattccacttacatcccaatgtcaaaacctccagcatcatagatttatttaatgacattccagaaaatgttagctgccgaaatactcctttttattctattcatcaacaattcttcaaagctgaatatagtgcccactataagcatgttttgatgaaaattgaatctttattatctgcagaagatagcattatcttcactcatcctcttcaactggaaatgtatcgtttagcgaataatgatatcaagtcaaaagccaaactaattgtacaaattcatggtaattatatggaagaaatccataactatgaaattttggcacgaaatatcgattatgttgactatcttcaaacggtatctgatgaaatgctggaagaaatgcattcccatttcaaaatcaaaaaagacaaattagtttttattccaaacatcacttatcccatttcattagaaaaaaaagaagctgatttctttattaaggataatgaagacatcgataatgctcagaaatttaaacgtatctctattgttggcagcattcagccaagaaaaaaccaattggatgccattaaaatcatcaataaaattaaaaatgaaaattacattttacagatatatggcaaatctattaataaagattactttgaattaattaaaaaatatattaaagacaataagttacaaaaccgtatcttattcaaaggtgaatcttccgagcaggaaatttatgaaaatacagatatcctgatcatgacatcagaaagtgagggatttccatatatatttatggaaggcatggtgtatgatattccaatcgttgtatatgattttaaatatggagcgaatgattacagtaactataatgaaaatggttgtgtttttaaaactggtgatatttctggaatggcaaaaaaaataattgagctattaaataacccagaaaaatataaagaattagttcaatataatcacaatcgcttcttaaaagaatatgcaaaagatgtggttatggctaaatatttcactattcttccgcgcagctttaataacgtatcattatcgtctgctttcagccgaaaagaattggacgaattccaaaatattactttttctattgaagattctaatgatttagctcatatttggaatttcgagctaaccaatcctgcacaaaatatgaatttttttgctttagttggcaagcgaaaatttccaatggatgctcatatccaaggaacacagtgtacgattaagatagctcataaaaagacagggaatttattgtcgcttttactaaaaaaacgaaatcagttgaatttatcaaggggatataccttaattgcagaagataatagctatgaaaaatatattggagcaatatctaataaaggtaactttgaaattattgcaaataaaaagagctcattagttactataaacaaaagtaccttagagttgcatgagattccccatgaactacatcagaataaattactgattgctttacccaacatgcaaacgcctctaaaaattactgatgataatttaatacctatccaagcctccataaaattagaaaagattggaaatacttattacccatgtttcttgccatctggcatatttaataatatctgcttagattacggtgaagaatccaaaattattaattttagtaaatattcttataaatatatctatgactcaattcgtcatattgagcaacatacagatatatcggatattatcgtttgcaatgtttattcttgggaacttattcgtgcctcagttattgagagccttatggaatttaccggaaaatgggaaaaacactttcagacttctcctaaaattgattatcgatttgatcatgaaggtaagcgttcgatggatgatgtcttttcagaagaaacatttattatggaatttccgcgtaaaaatggtatagataagaaaacagcagccttccaaaatataccaaacagtattgtaatggagtatccgcagaccaatggttacagtatgcgcagtcattcactgaaaagtaatgtagttgcggcaaaacattttcttgaaaaattaaataaaattaaggtagatattaaatttaaaaagcatgaccttgcaaacatcaaaaaaatgaatcgaattatttatgagcatttaggcattaacataaatatcgaagcatttctaaaaccacgattagaaaaatttaagcgtgaagaaaaatattttcatgatttcttcaaaagaaataattttaaagaggtaatttttccaagcacttattggaatccaggtattatttgtgctgcacataaacaaggtattaaggtatctgatattcaatatgctgccattactccttatcatcctgcgtattttaaatcaccaaaatcacattacgttgctgataaattgttcttatggtctgaatattggaatcatgagcttttaccaaatccaacacgagagattggttctggtgccgcatattggtatgcattagatgatgtgagattttcagaaaaactgaattatgactatatctttctatctcaaagtaggatttcttcgcgcttgcttagttttgcaattgagtttgcattaaaaaatcctcaactacagcttttattttctaagcatccagatgaaaatatagatttaaagaacagaattattcctgataatcttataatctccacggaatcttctatacaaggcatcaatgaatctcgcgttgctgtaggtgtttattcaactagcttatttgaggcattagcatgcggcaaacaaacttttgttgttaaatatccgggatatgaaattatgtcaaatgaaatagattcagggttattctttgcagtagaaacacctgaagaaatgcttgagaaaacaagcccgaattgggtggctgtggcagatattgaaaaccagttttttggccaagaaaaataa

SEQ ID NO: 2 - protein, 1037 aa, Neisseria meningitidis
MAVIIFVNGIRAVNGLVKSSINTANAFAEEGLDVHLINFVGNITGAEHLYPPFHLHPNVKTSSIIDLFNDIPENVSCRNTPFYSIHQQFFKAEYSAHYKHVLMKIESLLSAEDSIIFTHPLQLEMYRLANNDIKSKAKLIVQIHGNYMEEIHNYEILARNIDYVDYLQTVSDEMLEEMHSHFKIKKDKLVFIPNITYPISLEKKEADFFIKDNEDIDNAQKFKRISIVGSIQPRKNQLDAIKIINKIKNENYILQIYGKSINKDYFELIKKYIKDNKLQNRILFKGESSEQEIYENTDILIMTSESEGFPYIFMEGMVYDIPIVVYDFKYGANDYSNYNENGCVFKTGDISGMAKKIIELLNNPEKYKELVQYNHNRFLKEYAKDVVMAKYFTILPRSFNNVSLSSAFSRKELDEFQNITFSIEDSNDLAHIWNFELTNPAQNMNFFALVGKRKFPMDAHIQGTQCTIKIAHKKTGNLLSLLLKKRNQLNLSRGYTLIAEDNSYEKYIGAISNKGNFEIIANKKSSLVTINKSTLELHEIPHELHQNKLLIALPNMQTPLKITDDNLIPIQASIKLEKIGNTYYPCFLPSGIFNNICLDYGEESKIINFSKYSYKYIYDSIRHIEQHTDISDIIVCNVYSWELIRASVIESLMEFTGKWEKHFQTSPKIDYRFDHEGKRSMDDVFSEETFIMEFPRKNGIDKKTAAFQNIPNSIVMEYPQTNGYSMRSHSLKSNVVAAKHFLEKLNKIKVDIKFKKHDLANIKKMNRIIYEHLGININIEAFLKPRLEKFKREEKYFHDFFKRNNFKEVIFPSTYWNPGIICAAHKQGIKVSDIQYAAITPYHPAYFKSPKSHYVADKLFLWSEYWNHELLPNPTREIGSGAAYWYALDDVRFSEKLNYDYIFLSQSRISSRLLSFAIEFALKNPQLQLLFSKHPDENIDLKNRIIPDNLIISTESSIQGINESRVAVGVYSTSLFEALACGKQTFVVKYPGYEIMSNEIDSGLFFAVETPEEMLEKTSPNWVAVADIENQFFGQEK

SEQ ID NO: 3 - DNA, 3114 bp, Neisseria meningitidis
atggctgttattatatttgttaacggaattcgggctgtaaatggccttgttaaatcatctatcaatactgcaaacgcttttgctgaagaaggactggatgttcatttaattaattttgttggcaatattactggagcagagcatttatcccccccattccacttacatcccaatgtcaaaacctccagcatcatagatttatttaatgacattccagaaaatgttagctgccgaaatattcctttttattctatccatcaacaattcttcaaagccgaatacagtgcccactataagcatgttttgatgaaaattgaatctttattatctgaagaagatagcattatcttcactcatcctcttcaactggaaatgtatcgtttagcgaataataatattaagtcaaaagccaagctaattgtacaaattcatggtaactatatggaagaaatccataactatgaaatttgggcacgaaatatcgattatgttgattatcttcaaacggtatctgatgaaatgctggaagaaatgcattcccatttcaaaatcaaaaaagacaaattagtttttattccaaacatcacttatcccatttcattagaaaaaaaagaagctgatttctttattaaggataatgaagacattgataatgctcagaaatttaaacgtatctctattgttggcagtattcagccaagaaaaaaccaattggatgccattaaaatcatcaataaaattaaaaatgaaaattacattttacagatatatggcaaatctattaataaagattactttgaattaattaaaaaatatattaaagacaataagttacaaaaccgtatcttattcaaaggtgaatcttccgagcaggaaatttatgagaatacagatatcctaatcatgacatctcaaagcgaaggctttggttatatatttctagagggtatggtgtacgatatccctatccttgcctataattttaaatatggagcgaatgattttagcaattataatgaaaacgcttcagtttttaaaactggtgatatttctggaatggcaaaaaaaataattgagctattaaataacccagaaaaatataaagaattagttcaatataatcacaatcgcttcttaaaagaatatgcaaaagatgtggttatggctaaatatttcactattcttccgcgcagctttaataacgtatcattatcgtctgctttcagccgaaaagaattggacgaattccaaaatattactttttctattgaagattctaatgatttagctcatatttggaatttcgagctaaccaatcctgcacaaaatatgaatttttttgctttagttggcaagcgaaaatttccaatggatgctcatatccaaggaacacagtgtacgattaagatagctcataaaaagacagggaatttattgtcgcttttactaaaaaaacgaaatcagttgaatttatcaaggggatataccttaattgcagaagataatagctatgaaaaatatattggagcaatatctaataaaggtaactttgaaattattgcaaataaaaagaactcattagttactataaacaaaagtaccttagagttgcatgagattccccatgaactacatcagaataaattactgattgctttacccaacatgcaaacgcctctaaaaattactgatgataatttaatacctatccaagcctccataaaattagaaaagattggaaatacttattacccatgtttcttgccatctggcatatttaataatatctgcttagattacggtgaagaatccaaaattattaattttagtaaatattcttataaatatatctatgactcaattcgtcatattgagcaacatacagatatatcggatattatcgtttgcaatgtttattcttgggaacttattcgtgcctcagttattgagagccttatggaatttaccggaaaatgggaaaaacactttcagacttctcctaaaattgattatcgatttgatcatgaaggtaagcgttcgatggatgatgtcttttcagaagaaacatttattatggaatttccgcgtaaaaatggtatagataagaaaacagcagccttccaaaatataccaaacagtattgtaatggagtatccgcagaccaatggttacagtatgcgcagtcattcactgaaaagtaatgtagttgcggcaaaacattttcttgaaaaattaaataaaattaaggtagatattaaatttaaaaagcatgaccttgcaaacatcaaaaaaatgaatcgaattatttatgagcatttaggcattaacataaatatcgaagcatttctaaaaccacgattagaaaaatttaagcgtgaagaaaaatattttcatgatttcttcaaaagaaataattttaaagaggtaatttttccaagcacttattggaatccaggtattatttgtgctgcacataaacaaggtattaaggtatctgatattcaatatgctgccattactccttatcatcctgcgtattttaaatcaccaaaatcacattacgttgctgataaattgttcttatggtctgaatattggaatcatgagcttttaccaaatccaacacgagagattggttctggtgccgcatattggtatgcattagatgatgtgagattttcagaaaaactgaattatgactatatctttctatctcaaagtaggatttcttcgcgcttgcttagttttgcaattgagtttgcattaaaaaatcctcaactacagcttttattttctaagcatctagatgaaaatatagatttaaagaacagaattattcctgataatcttataatctccacggaatcttctatacaaggcatcaatgaatctcgcgttgctgtaggtgtttattcaactagcttatttgaggcattagcatgcggcaaacaaacttttgttgttaaatatccgggatatgaaattatgtcaaatgaaatagattcagggttattctttgcagtagaaacacctgaagaaatgcttgagaaaacaagcccgaattgggtggctgtggcagatattgaaaaccagttttttggccaagaaaaataa

SEQ ID NO: 4 - protein, 1037 aa, Neisseria meningitidis
MAVIIFVNGIRAVNGLVKSSINTANAFAEEGLDVHLINFVGNITGAEHLSPPFHLHPNVKTSSIIDLFNDIPENVSCRNIPFYSIHQQFFKAEYSAHYKHVLMKIESLLSEEDSIIFTHPLQLEMYRLANNNIKSKAKLIVQIHGNYMEEIHNYEIWARNIDYVDYLQTVSDEMLEEMHSHFKIKKDKLVFIPNITYPISLEKKEADFFIKDNEDIDNAQKFKRISIVGSIQPRKNQLDAIKIINKIKNENYILQIYGKSINKDYFELIKKYIKDNKLQNRILFKGESSEQEIYENTDILIMTSQSEGFGYIFLEGMVYDIPILAYNFKYGANDFSNYNENASVFKTGDISGMAKKIIELLNNPEKYKELVQYNHNRFLKEYAKDVVMAKYFTILPRSFNNVSLSSAFSRKELDEFQNITFSIEDSNDLAHIWNFELTNPAQNMNFFALVGKRKFPMDAHIQGTQCTIKIAHKKTGNLLSLLLKKRNQLNLSRGYTLIAEDNSYEKYIGAISNKGNFEIIANKKNSLVTINKSTLELHEIPHELHQNKLLIALPNMQTPLKITDDNLIPIQASIKLEKIGNTYYPCFLPSGIFNNICLDYGEESKIINFSKYSYKYIYDSIRHIEQHTDISDIIVCNVYSWELIRASVIESLMEFTGKWEKHFQTSPKIDYRFDHEGKRSMDDVFSEETFIMEFPRKNGIDKKTAAFQNIPNSIVMEYPQTNGYSMRSHSLKSNVVAAKHFLEKLNKIKVDIKFKKHDLANIKKMNRIIYEHLGININIEAFLKPRLEKFKREEKYFHDFFKRNNFKEVIFPSTYWNPGIICAAHKQGIKVSDIQYAAITPYHPAYFKSPKSHYVADKLFLWSEYWNHELLPNPTREIGSGAAYWYALDDVRFSEKLNYDYIFLSQSRISSRLLSFAIEFALKNPQLQLLFSKHLDENIDLKNRIIPDNLIISTESSIQGINESRVAVGVYSTSLFEALACGKQTFVVKYPGYEIMSNEIDSGLFFAVETPEEMLEKTSPNWVAVADIENQFFGQEK

SEQ ID NO: 5 - DNA, 1461 bp, Neisseria meningitidis
atgattatgagcaaaattagcaaattggtaacccacccaaaccttttctttcgagattatttcttaaaaaaagcaccgttaaattatggcgaaaatattaaacctttaccagtcgaaacctcttctcatagcaaaaaaaatacagcccataaaacacccgtatcatccgaccaaccaattgaagatccatacccagtaacatttccaattgatgtagtttatacttgggtagattcagatgatgaaaaattcaatgaagaacgcctaaagtttcaaaattcaagcacatctgagactctacaaggcaaagcagaaagcaccgatattgcaagattccaatcacgcgacgaattaaaatattcgattcgaagcctgatgaagtatgccccatgggtaaatcatatttacattgtaacaaatggtcaaataccaaaatggttagataccaacaatacaaaggtaacgattatccctcactcaactattatcgacagtcaatttctccctacttttaattctcacgtcattgaatcctctctatataaaatcccaggattatcagagcattacatttatttcaatgatgatgtcatgctagctagagatttaagcccatcttatttctttacaagcagcggattagcaaaactgtttattaccaactctcgtctaccaaatggctataagaatgtgaaagacacaccaacccaatgggcctcaaaaaattcccgtgagcttttacatgcagaaacaggattttgggctgaagccatgtttgcacatacttttcatccacaacgtaaaagtgtacatgaatctattgaacacctatggcatgaacaattaaatgtttgtcgtcaaaaccgtttccgtgatatttcagatattaacatggcgacattcctgcaccaccattttgccattttgacaggccaagctcttgctacacgcactaaatgtatttactttaacgttcgctctcctcaagcagctcagcattacaaaacattattagctcgaaaaggaagcgaatacagcccacattctatctgcttaaatgatcatacatcgagcaataaaaatattttatctaattacgaagccaaattacaaagctttttagaaacatactatccagatgtatcagaagcagaaattctccttcctactaaatctgaagtagctgaattagttaaacataaagattatttaactgtatatactaaattattacctattatcaataagcagctggtcaataaatataataaaccttattcatatcttttctattatttaggtttatctgcccggtttttatttgaagaaacgcaacaagaacactaccgggaaactgctgaagaaaatttacaaatcttttgtggcctaaacccaaaacatacactagccctcaaatacttagcggatgtcaccctcacatcacagcctagtggacaataa

SEQ ID NO: 6 - protein, 486 aa, Neisseria meningitidis
MIMSKISKLVTHPNLFFRDYFLKKAPLNYGENIKPLPVETSSHSKKNTAHKTPVSSDQPIEDPYPVTFPIDVVYTWVDSDDEKFNEERLKFQNSSTSETLQGKAESTDIARFQSRDELKYSIRSLMKYAPWVNHIYIVTNGQIPKWLDTNNTKVTIIPHSTIIDSQFLPTFNSHVIESSLYKIPGLSEHYIYFNDDVMLARDLSPSYFFTSSGLAKLFITNSRLPNGYKNVKDTPTQWASKNSRELLHAETGFWAEAMFAHTFHPQRKSVHESIEHLWHEQLNVCRQNRFRDISDINMATFLHHHFAILTGQALATRTKCIYFNVRSPQAAQHYKTLLARKGSEYSPHSICLNDHTSSNKNILSNYEAKLQSFLETYYPDVSEAEILLPTKSEVAELVKHKDYLTVYTKLLPIINKQLVNKYNKPYSYLFYYLGLSARFLFEETQQEHYRETAEENLQIFCGLNPKHTLALKYLADVTLTSQPSGQ

SEQ ID NO: 7 - DNA, 1638 bp, Neisseria meningitidis
atgtttatacttaataacagaaaatggcgtaaacttaaaagagaccctagcgctttctttcgagatagtaaatttaactttttaagatatttttctgctaaaaaatttgcaaagaattttaaaaattcatcacatatccataaaactaatataagtaaagctcaatcaaatatttcttcaaccttaaaacaaaatcggaaacaagatatgttaattcctattaatttttttaattttgaatatatagttaaaaaacttaacaatcaaaacgcaataggtgtatatattcttccttctaatcttactcttaagcctgcattatgtattctagaatcacataaagaagactttttaaataaatttcttcttactatttcctctgaaaatttaaagcttcaatacaaatttaatggacaaataaaaaatcctaagtccgtaaatgaaatttggacagatttatttagcattgctcatgttgacatgaaactcagcacagatagaactttaagttcatctatatctcaattttggttcagattagagttctgtaaagaagataaggattttatcttatttcctacagctaacagatattctagaaaactttggaagcactctattaaaaataatcaattatttaaagaaggcatacgaaactattcagaaatatcttcattaccctatgaagaagatcataattttgatattgatttagtatttacttgggtcaactcagaagataagaattggcaagagttatataaaaaatataagcccgactttaatagcgatgcaaccagtacatcaagattccttagtagagatgaattaaaattcgcattacgctcttgggaaatgaatggatccttcattcgaaaaatttttattgtctctaattgtgctcccccagcatggctagatttaaataaccctaaaattcaatgggtatatcacgaagaaattatgccacaaagtgcccttcctacttttagctcacatgctattgaaaccagcttgcaccatataccaggaattagtaactattttatttacagcaatgacgacttcctattaactaaaccattgaataaagacaatttcttctattcgaatggtattgcaaagttaagattagaagcatggggaaatgttaatggtgaatgtactgaaggagaacctgactacttaaatggtgctcgcaatgcgaacactctcttagaaaaggaatttaaaaaatttactactaaactacatactcactcccctcaatccatgagaactgatattttatttgagatggaaaaaaaatatccagaagagtttaatagaacactacataataaattccgatctttagatgatattgcagtaacgggctatctctatcatcattatgccctactctctggacgagcactacaaagttctgacaagacggaacttgtacagcaaaatcatgatttcaaaaagaaactaaataatgtagtgaccttaactaaagaaaggaattttgacaaacttcctttgagcgtatgtatcaacgatggtgctgatagtcacttgaatgaagaatggaatgttcaagttattaagttcttagaaactcttttcccattaccatcatcatttgagaaataa

SEQ ID NO: 8 - protein, 545 aa, Neisseria meningitidis
MFILNNRKWRKLKRDPSAFFRDSKFNFLRYFSAKKFAKNFKNSSHIHKTNISKAQSNISSTLKQNRKQDMLIPINFFNFEYIVKKLNNQNAIGVYILPSNLTLKPALCILESHKEDFLNKFLLTISSENLKLQYKFNGQIKNPKSVNEIWTDLFSIAHVDMKLSTDRTLSSSISQFWFRLEFCKEDKDFILFPTANRYSRKLWKHSIKNNQLFKEGIRNYSEISSLPYEEDHNFDIDLVFTWVNSEDKNWQELYKKYKPDFNSDATSTSRFLSRDELKFALRSWEMNGSFIRKIFIVSNCAPPAWLDLNNPKIQWVYHEEIMPQSALPTFSSHAIETSLHHIPGISNYFIYSNDDFLLTKPLNKDNFFYSNGIAKLRLEAWGNVNGECTEGEPDYLNGARNANTLLEKEFKKFTTKLHTHSPQSMRTDILFEMEKKYPEEFNRTLHNKFRSLDDIAVTGYLYHHYALLSGRALQSSDKTELVQQNHDFKKKLNNVVTLTKERNFDKLPLSVCINDGADSHLNEEWNVQVIKFLETLFPLPSSFEK

SEQ ID NO: 9 - DNA, 1893 bp, Leishmania major
atgacgaacccgtccaactccaacctgcaggccttgcgcgaggagctctgcacgcctggcctggatcagggtcacctcttcgagggatggccggagactgtggatgagtgcaacgagaggcagatcgccctcctcacagatttgtacatgttttccaacatgtatcccggcggcgttgctcagtacatccgcaacgggcacgagctgctggcgcgtgagagcgaagaggtggactttgcagcgctggagatgccccctctcatcttcgaggcgccgtcgctgcaccggcgcacggctgagaggacggcgctggagaacgccggaaccgcgatgctgtgcaagacggtgttcgtgctggttgctggcggtctgggcgaacgtctgggctactcgagcatcaaggtgagcctgccggtggagacggcgacgaacacaacgtatctcgcctactacctccggtgggcccagcgggtgggggggaaggaggtaccatttgtgataatgacctctgacgacacgcacgaccgcacgctgcagctcctgcgcgagctgcagttggaggtgcccaacttgcatgtgctcaagcaggggcaggtcttctgttttgccgacagcgccgcgcacctcgccctggacgagacagggaagctgctgcgcaagccacacggtcacggcgacgtgcactccctcatctacaacgcgactgtgaagagagacgtggtgccggactccggcgacggtaccgcgacggcgcagccactcgtgaacgactggctggcggccggctacgagtccattgtcttcatccaggacaccaacgccggcgcgacgatcacaatccccatcagcctcgccttgagtgccgagcactcgctcgacatgaacttcacctgcatccctcgtgtgccgaaggagccgatcgggctgctatgccgaaccaagaagaatagcggcgacccgtggctggtcgcgaacgtggagtacaacgtctttgccgaggtctcgcgcgcgcttaacaaggatggtggcgatgaagtcagtgaccccactggcttctccccgttccctggcagcgtcaacaccctcgtgttcaagctctccagctacgtggaccggctgcgggagtcgcacggtatcgtgccggagttcatcaatcccaagtactcggacgagacgcgccgctccttcaagaagcccgcacgcatcgagtccctgatgcaggacatcgcgctgctcttctccgaggatgactaccgtgtcggcggtaccgtctttgagcgattctcgtaccagccagtgaagaactcgctagaggaggcggcagggcttgtggcgcagggcaacggcgcctactgcgccgccacgggagaggctgccttctacgagctgcagcggcgccgtctcaaggccatcgggctgccgctcttctacagctcgcagccggaggtgacggtggcgaaggacgcctttggcgtgcgtctcttcccgataatcgtgctggatacgatgtgcgcgtcaagcggatccctcgacgaccttgcgcgcgtctttccgacgccggaaaaggtgcacatcgatcagcacagcaccttgattgttgagggccgtgtcatcatcgagagcctggagctatacggtgcactcacgattcgcggcccgacagactcgatggcgctgccgcacgtagtacgaaacgctgtggtgcgcaatgccggctggtcggtacacgcgatcttgtctctctgcgctgggcgcgatagcaggctgtccgaggtggaccgcatccgcgggtttgtgctgaagaagacagccatggcggtgatggactgcaatacgaagggcgagtccgaggccggtgcaccgtctggtgcggctgacccggcaaagttgtag

SEQ ID NO: 10 - protein, 630 aa, Leishmania major
MTNPSNSNLQALREELCTPGLDQGHLFEGWPETVDECNERQIALLTDLYMFSNMYPGGVAQYIRNGHELLARESEEVDFAALEMPPLIFEAPSLHRRTAERTALENAGTAMLCKTVFVLVAGGLGERLGYSSIKVSLPVETATNTTYLAYYLRWAQRVGGKEVPFVIMTSDDTHDRTLQLLRELQLEVPNLHVLKQGQVFCFADSAAHLALDETGKLLRKPHGHGDVHSLIYNATVKRDVVPDSGDGTATAQPLVNDWLAAGYESIVFIQDTNAGATITIPISLALSAEHSLDMNFTCIPRVPKEPIGLLCRTKKNSGDPWLVANVEYNVFAEVSRALNKDGGDEVSDPTGFSPFPGSVNTLVFKLSSYVDRLRESHGIVPEFINPKYSDETRRSFKKPARIESLMQDIALLFSEDDYRVGGTVFERFSYQPVKNSLEEAAGLVAQGNGAYCAATGEAAFYELQRRRLKAIGLPLFYSSQPEVTVAKDAFGVRLFPIIVLDTMCASSGSLDDLARVFPTPEKVHIDQHSTLIVEGRVIIESLELYGALTIRGPTDSMALPHVVRNAVVRNAGWSVHAILSLCAGRDSRLSEVDRIRGFVLKKTAMAVMDCNTKGESEAGAPSGAADPAKL

SEQ ID NO: 11 - DNA, 1119 bp, Neisseria meningitidis
atgaaagtcttaaccgtctttggcactcgccctgaagctattaaaatggcgcctgtaattctagagttacaaaaacataacacaattacttcaaaagtttgcattactgcacagcatcgtgaaatgctagatcaggttttgagcctattcgaaatcaaagctgattatgatttaaatatcatgaaacccaaccagagcctacaagaaatcacaacaaatatcatctcaagccttaccgatgttcttgaagatttcaaacctgactgcgtccttgctcacggagacaccacaacaacttttgcagctagccttgctgcattctatcaaaaaatacctgttggccacattgaagcaggcctgagaacttataatttatactctccttggccagaggaagcaaataggcgtttaacaagcgttctaagccagtggcattttgcacctactgaagattctaaaaataacttactatctgaatcaataccttctgacaaagttattgttactggaaatactgtcatagatgcactaatggtatctctagaaaaactaaaaataactacaattaaaaaacaaatggaacaagcttttccatttattcaggacaactctaaagtaattttaattaccgctcatagaagagaaaatcatggggaaggtattaaaaatattggactttctatcttagaattagctaaaaaatacccaacattctcttttgtgattccgctccatttaaatcctaacgttagaaaaccaattcaagatttattatcctctgtgcacaatgttcatcttattgagccacaagaatacttaccattcgtatatttaatgtctaaaagccatataatattaagtgattcaggcggcatacaagaagaagctccatccctaggaaaaccagttcttgtattaagagatactacagaacgtcctgaagctgtagctgcaggaactgtaaaattagtaggttctgaaactcaaaatattattgagagctttacacaactaattgaataccctgaatattatgaaaaaatggctaatattgaaaacccttacgggataggtaatgcctcaaaaatcattgtagaaactttattaaagaatagataa

SEQ ID NO: 12 - protein, 372 aa, Neisseria meningitidis
MKVLTVFGTRPEAIKMAPVILELQKHNTITSKVCITAQHREMLDQVLSLFEIKADYDLNIMKPNQSLQEITTNIISSLTDVLEDFKPDCVLAHGDTTTTFAASLAAFYQKIPVGHIEAGLRTYNLYSPWPEEANRRLTSVLSQWHFAPTEDSKNNLLSESIPSDKVIVTGNTVIDALMVSLEKLKITTIKKQMEQAFPFIQDNSKVILITAHRRENHGEGIKNIGLSILELAKKYPTFSFVIPLHLNPNVRKPIQDLLSSVHNVHLIEPQEYLPFVYLMSKSHIILSDSGGIQEEAPSLGKPVLVLRDTTERPEAVAAGTVKLVGSETQNIIESFTQLIEYPEYYEKMANIENPYGIGNASKIIVETLLKNR

SEQ ID NO: 13 - DNA, 3192 bp, Neisseria meningitidis
atggctagctggagccacccgcagttcgaaaaaggcgccctggttccgcgtggatccgctgttattatatttgttaacggaattcgggctgtaaatggccttgttaaatcatctatcaatactgcaaacgcttttgctgaagaaggactggatgttcatttaattaattttgttggcaatattactggagcagagcatttataccccccattccacttacatcccaatgtcaaaacctccagcatcatagatttatttaatgacattccagaaaatgttagctgccgaaatactcctttttattctattcatcaacaattcttcaaagctgaatatagtgcccactataagcatgttttgatgaaaattgaatctttattatctgcagaagatagcattatcttcactcatcctcttcaactggaaatgtatcgtttagcgaataatgatatcaagtcaaaagccaaactaattgtacaaattcatggtaattatatggaagaaatccataactatgaaattttggcacgaaatatcgattatgttgactatcttcaaacggtatctgatgaaatgctggaagaaatgcattcccatttcaaaatcaaaaaagacaaattagtttttattccaaacatcacttatcccatttcattagaaaaaaaagaagctgatttctttattaaggataatgaagacatcgataatgctcagaaatttaaacgtatctctattgttggcagcattcagccaagaaaaaaccaattggatgccattaaaatcatcaataaaattaaaaatgaaaattacattttacagatatatggcaaatctattaataaagattactttgaattaattaaaaaatatattaaagacaataagttacaaaaccgtatcttattcaaaggtgaatcttccgagcaggaaatttatgaaaatacagatatcctgatcatgacatcagaaagtgagggatttccatatatatttatggaaggcatggtgtatgatattccaatcgttgtatatgattttaaatatggagcgaatgattacagtaactataatgaaaatggttgtgtttttaaaactggtgatatttctggaatggcaaaaaaaataattgagctattaaataacccagaaaaatataaagaattagttcaatataatcacaatcgcttcttaaaagaatatgcaaaagatgtggttatggctaaatatttcactattcttccgcgcagctttaataacgtatcattatcgtctgctttcagccgaaaagaattggacgaattccaaaatattactttttctattgaagattctaatgatttagctcatatttggaatttcgagctaaccaatcctgcacaaaatatgaatttttttgctttagttggcaagcgaaaatttccaatggatgctcatatccaaggaacacagtgtacgattaagatagctcataaaaagacagggaatttattgtcgcttttactaaaaaaacgaaatcagttgaatttatcaaggggatataccttaattgcagaagataatagctatgaaaaatatattggagcaatatctaataaaggtaactttgaaattattgcaaataaaaagagctcattagttactataaacaaaagtaccttagagttgcatgagattccccatgaactacatcagaataaattactgattgctttacccaacatgcaaacgcctctaaaaattactgatgataatttaatacctatccaagcctccataaaattagaaaagattggaaatacttattacccatgtttcttgccatctggcatatttaataatatctgcttagattacggtgaagaatccaaaattattaattttagtaaatattcttataaatatatctatgactcaattcgtcatattgagcaacatacagatatatcggatattatcgtttgcaatgtttattcttgggaacttattcgtgcctcagttattgagagccttatggaatttaccggaaaatgggaaaaacactttcagacttctcctaaaattgattatcgatttgatcatgaaggtaagcgttcgatggatgatgtcttttcagaagaaacatttattatggaatttccgcgtaaaaatggtatagataagaaaacagcagccttccaaaatataccaaacagtattgtaatggagtatccgcagaccaatggttacagtatgcgcagtcattcactgaaaagtaatgtagttgcggcaaaacattttcttgaaaaattaaataaaattaaggtagatattaaatttaaaaagcatgaccttgcaaacatcaaaaaaatgaatcgaattatttatgagcatttaggcattaacataaatatcgaagcatttctaaaaccacgattagaaaaatttaagcgtgaagaaaaatattttcatgatttcttcaaaagaaataattttaaagaggtaatttttccaagcacttattggaatccaggtattatttgtgctgcacataaacaaggtattaaggtatctgatattcaatatgctgccattactccttatcatcctgcgtattttaaatcaccaaaatcacattacgttgctgataaattgttcttatggtctgaatattggaatcatgagcttttaccaaatccaacacgagagattggttctggtgccgcatattggtatgcattagatgatgtgagattttcagaaaaactgaattatgactatatctttctatctcaaagtaggatttcttcgcgcttgcttagttttgcaattgagtttgcattaaaaaatcctcaactacagcttttattttctaagcatccagatgaaaatatagatttaaagaacagaattattcctgataatcttataatctccacggaatcttctatacaaggcatcaatgaatctcgcgttgctgtaggtgtttattcaactagcttatttgaggcattagcatgcggcaaacaaacttttgttgttaaatatccgggatatgaaattatgtcaaatgaaatagattcagggttattctttgcagtagaaacacctgaagaaatgcttgagaaaacaagcccgaattgggtggctgtggcagatattgaaaaccagttttttggccaagaaaaactcgagcaccaccaccaccaccactga

SEQ ID NO: 14 - protein, 1063 aa, Neisseria meningitidis
MASWSHPQFEKGALVPRGSAVIIFVNGIRAVNGLVKSSINTANAFAEEGLDVHLINFVGNITGAEHLYPPFHLHPNVKTSSIIDLFNDIPENVSCRNTPFYSIHQQFFKAEYSAHYKHVLMKIESLLSAEDSIIFTHPLQLEMYRLANNDIKSKAKLIVQIHGNYMEEIHNYEILARNIDYVDYLQTVSDEMLEEMHSHFKIKKDKLVFIPNITYPISLEKKEADFFIKDNEDIDNAQKFKRISIVGSIQPRKNQLDAIKIINKIKNENYILQIYGKSINKDYFELIKKYIKDNKLQNRILFKGESSEQEIYENTDILIMTSESEGFPYIFMEGMVYDIPIVVYDFKYGANDYSNYNENGCVFKTGDISGMAKKIIELLNNPEKYKELVQYNHNRFLKEYAKDVVMAKYFTILPRSFNNVSLSSAFSRKELDEFQNITFSIEDSNDLAHIWNFELTNPAQNMNFFALVGKRKFPMDAHIQGTQCTIKIAHKKTGNLLSLLLKKRNQLNLSRGYTLIAEDNSYEKYIGAISNKGNFEIIANKKSSLVTINKSTLELHEIPHELHQNKLLIALPNMQTPLKITDDNLIPIQASIKLEKIGNTYYPCFLPSGIFNNICLDYGEESKIINFSKYSYKYIYDSIRHIEQHTDISDIIVCNVYSWELIRASVIESLMEFTGKWEKHFQTSPKIDYRFDHEGKRSMDDVFSEETFIMEFPRKNGIDKKTAAFQNIPNSIVMEYPQTNGYSMRSHSLKSNVVAAKHFLEKLNKIKVDIKFKKHDLANIKKMNRIIYEHLGININIEAFLKPRLEKFKREEKYFHDFFKRNNFKEVIFPSTYWNPGIICAAHKQGIKVSDIQYAAITPYHPAYFKSPKSHYVADKLFLWSEYWNHELLPNPTREIGSGAAYWYALDDVRFSEKLNYDYIFLSQSRISSRLLSFAIEFALKNPQLQLLFSKHPDENIDLKNRIIPDNLIISTESSIQGINESRVAVGVYSTSLFEALACGKQTFVVKYPGYEIMSNEIDSGLFFAVETPEEMLEKTSPNWVAVADIENQFFGQEKLEHHHHHH

SEQ ID NO: 15 - DNA, 3192 bp, Neisseria meningitidis
atggctagctggagccacccgcagttcgaaaaaggcgccctggttccgcgtggatccgctgttattatatttgttaacggaattcgggctgtaaatggccttgttaaatcatctatcaatactgcaaacgcttttgctgaagaaggactggatgttcatttaattaattttgttggcaatattactggagcagagcatttatcccccccattccacttacatcccaatgtcaaaacctccagcatcatagatttatttaatgacattccagaaaatgttagctgccgaaatattcctttttattctatccatcaacaattcttcaaagccgaatacagtgcccactataagcatgttttgatgaaaattgaatctttattatctgaagaagatagcattatcttcactcatcctcttcaactggaaatgtatcgtttagcgaataataatattaagtcaaaagccaagctaattgtacaaattcatggtaactatatggaagaaatccataactatgaaatttgggcacgaaatatcgattatgttgattatcttcaaacggtatctgatgaaatgctggaagaaatgcattcccatttcaaaatcaaaaaagacaaattagtttttattccaaacatcacttatcccatttcattagaaaaaaaagaagctgatttctttattaaggataatgaagacattgataatgctcagaaatttaaacgtatctctattgttggcagtattcagccaagaaaaaaccaattggatgccattaaaatcatcaataaaattaaaaatgaaaattacattttacagatatatggcaaatctattaataaagattactttgaattaattaaaaaatatattaaagacaataagttacaaaaccgtatcttattcaaaggtgaatcttccgagcaggaaatttatgagaatacagatatcctaatcatgacatctcaaagcgaaggctttggttatatatttctagagggtatggtgtacgatatccctatccttgcctataattttaaatatggagcgaatgattttagcaattataatgaaaacgcttcagtttttaaaactggtgatatttctggaatggcaaaaaaaataattgagctattaaataacccagaaaaatataaagaattagttcaatataatcacaatcgcttcttaaaagaatatgcaaaagatgtggttatggctaaatatttcactattcttccgcgcagctttaataacgtatcattatcgtctgctttcagccgaaaagaattggacgaattccaaaatattactttttctattgaagattctaatgatttagctcatatttggaatttcgagctaaccaatcctgcacaaaatatgaatttttttgctttagttggcaagcgaaaatttccaatggatgctcatatccaaggaacacagtgtacgattaagatagctcataaaaagacagggaatttattgtcgcttttactaaaaaaacgaaatcagttgaatttatcaaggggatataccttaattgcagaagataatagctatgaaaaatatattggagcaatatctaataaaggtaactttgaaattattgcaaataaaaagaactcattagttactataaacaaaagtaccttagagttgcatgagattccccatgaactacatcagaataaattactgattgctttacccaacatgcaaacgcctctaaaaattactgatgataatttaatacctatccaagcctccataaaattagaaaagattggaaatacttattacccatgtttcttgccatctggcatatttaataatatctgcttagattacggtgaagaatccaaaattattaattttagtaaatattcttataaatatatctatgactcaattcgtcatattgagcaacatacagatatatcggatattatcgtttgcaatgtttattcttgggaacttattcgtgcctcagttattgagagccttatggaatttaccggaaaatgggaaaaacactttcagacttctcctaaaattgattatcgatttgatcatgaaggtaagcgttcgatggatgatgtcttttcagaagaaacatttattatggaatttccgcgtaaaaatggtatagataagaaaacagcagccttccaaaatataccaaacagtattgtaatggagtatccgcagaccaatggttacagtatgcgcagtcattcactgaaaagtaatgtagttgcggcaaaacattttcttgaaaaattaaataaaattaaggtagatattaaatttaaaaagcatgaccttgcaaacatcaaaaaaatgaatcgaattatttatgagcatttaggcattaacataaatatcgaagcatttctaaaaccacgattagaaaaatttaagcgtgaagaaaaatattttcatgatttcttcaaaagaaataattttaaagaggtaatttttccaagcacttattggaatccaggtattatttgtgctgcacataaacaaggtattaaggtatctgatattcaatatgctgccattactccttatcatcctgcgtattttaaatcaccaaaatcacattacgttgctgataaattgttcttatggtctgaatattggaatcatgagcttttaccaaatccaacacgagagattggttctggtgccgcatattggtatgcattagatgatgtgagattttcagaaaaactgaattatgactatatctttctatctcaaagtaggatttcttcgcgcttgcttagttttgcaattgagtttgcattaaaaaatcctcaactacagcttttattttctaagcatctagatgaaaatatagatttaaagaacagaattattcctgataatcttataatctccacggaatcttctatacaaggcatcaatgaatctcgcgttgctgtaggtgtttattcaactagcttatttgaggcattagcatgcggcaaacaaacttttgttgttaaatatccgggatatgaaattatgtcaaatgaaatagattcagggttattctttgcagtagaaacacctgaagaaatgcttgagaaaacaagcccgaattgggtggctgtggcagatattgaaaaccagttttttggccaagaaaaactcgagcaccaccaccaccaccactga

SEQ ID NO: 16 - protein, 1063 aa, Neisseria meningitidis
MASWSHPQFEKGALVPRGSAVIIFVNGIRAVNGLVKSSINTANAFAEEGLDVHLINFVGNITGAEHLSPPFHLHPNVKTSSIIDLFNDIPENVSCRNIPFYSIHQQFFKAEYSAHYKHVLMKIESLLSEEDSIIFTHPLQLEMYRLANNNIKSKAKLIVQIHGNYMEEIHNYEIWARNIDYVDYLQTVSDEMLEEMHSHFKIKKDKLVFIPNITYPISLEKKEADFFIKDNEDIDNAQKFKRISIVGSIQPRKNQLDAIKIINKIKNENYILQIYGKSINKDYFELIKKYIKDNKLQNRILFKGESSEQEIYENTDILIMTSQSEGFGYIFLEGMVYDIPILAYNFKYGANDFSNYNENASVFKTGDISGMAKKIIELLNNPEKYKELVQYNHNRFLKEYAKDVVMAKYFTILPRSFNNVSLSSAFSRKELDEFQNITFSIEDSNDLAHIWNFELTNPAQNMNFFALVGKRKFPMDAHIQGTQCTIKIAHKKTGNLLSLLLKKRNQLNLSRGYTLIAEDNSYEKYIGAISNKGNFEIIANKKNSLVTINKSTLELHEIPHELHQNKLLIALPNMQTPLKITDDNLIPIQASIKLEKIGNTYYPCFLPSGIFNNICLDYGEESKIINFSKYSYKYIYDSIRHIEQHTDISDIIVCNVYSWELIRASVIESLMEFTGKWEKHFQTSPKIDYRFDHEGKRSMDDVFSEETFIMEFPRKNGIDKKTAAFQNIPNSIVMEYPQTNGYSMRSHSLKSNVVAAKHFLEKLNKIKVDIKFKKHDLANIKKMNRIIYEHLGININIEAFLKPRLEKFKREEKYFHDFFKRNNFKEVIFPSTYWNPGIICAAHKQGIKVSDIQYAAITPYHPAYFKSPKSHYVADKLFLWSEYWNHELLPNPTREIGSGAAYWYALDDVRFSEKLNYDYIFLSQSRISSRLLSFAIEFALKNPQLQLLFSKHLDENIDLKNRIIPDNLIISTESSIQGINESRVAVGVYSTSLFEALACGKQTFVVKYPGYEIMSNEIDSGLFFAVETPEEMLEKTSPNWVAVADIENQFFGQEKLEHHHHHH

SEQ ID NO: 17 - DNA, 3138 bp, Neisseria meningitidis
atggctgttattatatttgttaacggaattcgggctgtaaatggccttgttaaatcatctatcaatactgcaaacgcttttgctgaagaaggactggatgttcatttaattaattttgttggcaatattactggagcagagcatttataccccccattccacttacatcccaatgtcaaaacctccagcatcatagatttatttaatgacattccagaaaatgttagctgccgaaatactcctttttattctattcatcaacaattcttcaaagctgaatatagtgcccactataagcatgttttgatgaaaattgaatctttattatctgcagaagatagcattatcttcactcatcctcttcaactggaaatgtatcgtttagcgaataatgatatcaagtcaaaagccaaactaattgtacaaattcatggtaattatatggaagaaatccataactatgaaattttggcacgaaatatcgattatgttgactatcttcaaacggtatctgatgaaatgctggaagaaatgcattcccatttcaaaatcaaaaaagacaaattagtttttattccaaacatcacttatcccatttcattagaaaaaaaagaagctgatttctttattaaggataatgaagacatcgataatgctcagaaatttaaacgtatctctattgttggcagcattcagccaagaaaaaaccaattggatgccattaaaatcatcaataaaattaaaaatgaaaattacattttacagatatatggcaaatctattaataaagattactttgaattaattaaaaaatatattaaagacaataagttacaaaaccgtatcttattcaaaggtgaatcttccgagcaggaaatttatgaaaatacagatatcctgatcatgacatcagaaagtgagggatttccatatatatttatggaaggcatggtgtatgatattccaatcgttgtatatgattttaaatatggagcgaatgattacagtaactataatgaaaatggttgtgtttttaaaactggtgatatttctggaatggcaaaaaaaataattgagctattaaataacccagaaaaatataaagaattagttcaatataatcacaatcgcttcttaaaagaatatgcaaaagatgtggttatggctaaatatttcactattcttccgcgcagctttaataacgtatcattatcgtctgctttcagccgaaaagaattggacgaattccaaaatattactttttctattgaagattctaatgatttagctcatatttggaatttcgagctaaccaatcctgcacaaaatatgaatttttttgctttagttggcaagcgaaaatttccaatggatgctcatatccaaggaacacagtgtacgattaagatagctcataaaaagacagggaatttattgtcgcttttactaaaaaaacgaaatcagttgaatttatcaaggggatataccttaattgcagaagataatagctatgaaaaatatattggagcaatatctaataaaggtaactttgaaattattgcaaataaaaagagctcattagttactataaacaaaagtaccttagagttgcatgagattccccatgaactacatcagaataaattactgattgctttacccaacatgcaaacgcctctaaaaattactgatgataatttaatacctatccaagcctccataaaattagaaaagattggaaatacttattacccatgtttcttgccatctggcatatttaataatatctgcttagattacggtgaagaatccaaaattattaattttagtaaatattcttataaatatatctatgactcaattcgtcatattgagcaacatacagatatatcggatattatcgtttgcaatgtttattcttgggaacttattcgtgcctcagttattgagagccttatggaatttaccggaaaatgggaaaaacactttcagacttctcctaaaattgattatcgatttgatcatgaaggtaagcgttcgatggatgatgtcttttcagaagaaacatttattatggaatttccgcgtaaaaatggtatagataagaaaacagcagccttccaaaatataccaaacagtattgtaatggagtatccgcagaccaatggttacagtatgcgcagtcattcactgaaaagtaatgtagttgcggcaaaacattttcttgaaaaattaaataaaattaaggtagatattaaatttaaaaagcatgaccttgcaaacatcaaaaaaatgaatcgaattatttatgagcatttaggcattaacataaatatcgaagcatttctaaaaccacgattagaaaaatttaagcgtgaagaaaaatattttcatgatttcttcaaaagaaataattttaaagaggtaatttttccaagcacttattggaatccaggtattatttgtgctgcacataaacaaggtattaaggtatctgatattcaatatgctgccattactccttatcatcctgcgtattttaaatcaccaaaatcacattacgttgctgataaattgttcttatggtctgaatattggaatcatgagcttttaccaaatccaacacgagagattggttctggtgccgcatattggtatgcattagatgatgtgagattttcagaaaaactgaattatgactatatctttctatctcaaagtaggatttcttcgcgcttgcttagttttgcaattgagtttgcattaaaaaatcctcaactacagcttttattttctaagcatccagatgaaaatatagatttaaagaacagaattattcctgataatcttataatctccacggaatcttctatacaaggcatcaatgaatctcgcgttgctgtaggtgtttattcaactagcttatttgaggcattagcatgcggcaaacaaacttttgttgttaaatatccgggatatgaaattatgtcaaatgaaatagattcagggttattctttgcagtagaaacacctgaagaaatgcttgagaaaacaagcccgaattgggtggctgtggcagatattgaaaaccagttttttggccaagaaaaactcgagcaccaccaccaccaccactga

SEQ ID NO: 18 - protein, 1045 aa, Neisseria meningitidis
MAVIIFVNGIRAVNGLVKSSINTANAFAEEGLDVHLINFVGNITGAEHLYPPFHLHPNVKTSSIIDLFNDIPENVSCRNTPFYSIHQQFFKAEYSAHYKHVLMKIESLLSAEDSIIFTHPLQLEMYRLANNDIKSKAKLIVQIHGNYMEEIHNYEILARNIDYVDYLQTVSDEMLEEMHSHFKIKKDKLVFIPNITYPISLEKKEADFFIKDNEDIDNAQKFKRISIVGSIQPRKNQLDAIKIINKIKNENYILQIYGKSINKDYFELIKKYIKDNKLQNRILFKGESSEQEIYENTDILIMTSESEGFPYIFMEGMVYDIPIVVYDFKYGANDYSNYNENGCVFKTGDISGMAKKIIELLNNPEKYKELVQYNHNRFLKEYAKDVVMAKYFTILPRSFNNVSLSSAFSRKELDEFQNITFSIEDSNDLAHIWNFELTNPAQNMNFFALVGKRKFPMDAHIQGTQCTIKIAHKKTGNLLSLLLKKRNQLNLSRGYTLIAEDNSYEKYIGAISNKGNFEIIANKKSSLVTINKSTLELHEIPHELHQNKLLIALPNMQTPLKITDDNLIPIQASIKLEKIGNTYYPCFLPSGIFNNICLDYGEESKIINFSKYSYKYIYDSIRHIEQHTDISDIIVCNVYSWELIRASVIESLMEFTGKWEKHFQTSPKIDYRFDHEGKRSMDDVFSEETFIMEFPRKNGIDKKTAAFQNIPNSIVMEYPQTNGYSMRSHSLKSNVVAAKHFLEKLNKIKVDIKFKKHDLANIKKMNRIIYEHLGININIEAFLKPRLEKFKREEKYFHDFFKRNNFKEVIFPSTYWNPGIICAAHKQGIKVSDIQYAAITPYHPAYFKSPKSHYVADKLFLWSEYWNHELLPNPTREIGSGAAYWYALDDVRFSEKLNYDYIFLSQSRISSRLLSFAIEFALKNPQLQLLFSKHPDENIDLKNRIIPDNLIISTESSIQGINESRVAVGVYSTSLFEALACGKQTFVVKYPGYEIMSNEIDSGLFFAVETPEEMLEKTSPNWVAVADIENQFFGQEKLEHHHHHH

SEQ ID NO: 19 - DNA, 2610 bp, Neisseria meningitidis
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtacccggccggggatccattatgagcaaaattagcaaattggtaacccacccaaaccttttctttcgagattatttcttaaaaaaagcaccgttaaattatggcgaaaatattaaacctttaccagtcgaaacctcttctcatagcaaaaaaaatacagcccataaaacacccgtatcatccgaccaaccaattgaagatccatacccagtaacatttccaattgatgtagtttatacttgggtagattcagatgatgaaaaattcaatgaagaacgcctaaagtttcaaaattcaagcacatctgagactctacaaggcaaagcagaaagcaccgatattgcaagattccaatcacgcgacgaattaaaatattcgattcgaagcctgatgaagtatgccccatgggtaaatcatatttacattgtaacaaatggtcaaataccaaaatggttagataccaacaatacaaaggtaacgattatccctcactcaactattatcgacagtcaatttctccctacttttaattctcacgtcattgaatcctctctatataaaatcccaggattatcagagcattacatttatttcaatgatgatgtcatgctagctagagatttaagcccatcttatttctttacaagcagcggattagcaaaactgtttattaccaactctcgtctaccaaatggctataagaatgtgaaagacacaccaacccaatgggcctcaaaaaattcccgtgagcttttacatgcagaaacaggattttgggctgaagccatgtttgcacatacttttcatccacaacgtaaaagtgtacatgaatctattgaacacctatggcatgaacaattaaatgtttgtcgtcaaaaccgtttccgtgatatttcagatattaacatggcgacattcctgcaccaccattttgccattttgacaggccaagctcttgctacacgcactaaatgtatttactttaacgttcgctctcctcaagcagctcagcattacaaaacattattagctcgaaaaggaagcgaatacagcccacattctatctgcttaaatgatcatacatcgagcaataaaaatattttatctaattacgaagccaaattacaaagctttttagaaacatactatccagatgtatcagaagcagaaattctccttcctactaaatctgaagtagctgaattagttaaacataaagattatttaactgtatatactaaattattacctattatcaataagcagctggtcaataaatataataaaccttattcatatcttttctattatttaggtttatctgcccggtttttatttgaagaaacgcaacaagaacactaccgggaaactgctgaagaaaatttacaaatcttttgtggcctaaacccaaaacatacactagccctcaaatacttagcggatgtcaccctcacatcacagcctagtggacaactcgagcaccaccaccaccaccac

SEQ ID NO: 20 - protein, 870 aa, Neisseria meningitidis
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPGRGSIMSKISKLVTHPNLFFRDYFLKKAPLNYGENIKPLPVETSSHSKKNTAHKTPVSSDQPIEDPYPVTFPIDVVYTWVDSDDEKFNEERLKFQNSSTSETLQGKAESTDIARFQSRDELKYSIRSLMKYAPWVNHIYIVTNGQIPKWLDTNNTKVTIIPHSTIIDSQFLPTFNSHVIESSLYKIPGLSEHYIYFNDDVMLARDLSPSYFFTSSGLAKLFITNSRLPNGYKNVKDTPTQWASKNSRELLHAETGFWAEAMFAHTFHPQRKSVHESIEHLWHEQLNVCRQNRFRDISDINMATFLHHHFAILTGQALATRTKCIYFNVRSPQAAQHYKTLLARKGSEYSPHSICLNDHTSSNKNILSNYEAKLQSFLETYYPDVSEAEILLPTKSEVAELVKHKDYLTVYTKLLPIINKQLVNKYNKPYSYLFYYLGLSARFLFEETQQEHYRETAEENLQIFCGLNPKHTLALKYLADVTLTSQPSGQLEHHHHHH

SEQ ID NO: 21 - DNA, 1716 bp, Neisseria meningitidis
atggctagctggagccacccgcagttcgaaaaaggcgccctggttccgcgtggatcttttatacttaataacagaaaatggcgtaaacttaaaagagaccctagcgctttctttcgagatagtaaatttaactttttaagatatttttctgctaaaaaatttgcaaagaattttaaaaattcatcacatatccataaaactaatataagtaaagctcaatcaaatatttcttcaaccttaaaacaaaatcggaaacaagatatgttaattcctattaatttttttaattttgaatatatagttaaaaaacttaacaatcaaaacgcaataggtgtatatattcttccttctaatcttactcttaagcctgcattatgtattctagaatcacataaagaagactttttaaataaatttcttcttactatttcctctgaaaatttaaagcttcaatacaaatttaatggacaaataaaaaatcctaagtccgtaaatgaaatttggacagatttatttagcattgctcatgttgacatgaaactcagcacagatagaactttaagttcatctatatctcaattttggttcagattagagttctgtaaagaagataaggattttatcttatttcctacagctaacagatattctagaaaactttggaagcactctattaaaaataatcaattatttaaagaaggcatacgaaactattcagaaatatcttcattaccctatgaagaagatcataattttgatattgatttagtatttacttgggtcaactcagaagataagaattggcaagagttatataaaaaatataagcccgactttaatagcgatgcaaccagtacatcaagattccttagtagagatgaattaaaattcgcattacgctcttgggaaatgaatggatccttcattcgaaaaatttttattgtctctaattgtgctcccccagcatggctagatttaaataaccctaaaattcaatgggtatatcacgaagaaattatgccacaaagtgcccttcctacttttagctcacatgctattgaaaccagcttgcaccatataccaggaattagtaactattttatttacagcaatgacgacttcctattaactaaaccattgaataaagacaatttcttctattcgaatggtattgcaaagttaagattagaagcatggggaaatgttaatggtgaatgtactgaaggagaacctgactacttaaatggtgctcgcaatgcgaacactctcttagaaaaggaatttaaaaaatttactactaaactacatactcactcccctcaatccatgagaactgatattttatttgagatggaaaaaaaatatccagaagagtttaatagaacactacataataaattccgatctttagatgatattgcagtaacgggctatctctatcatcattatgccctactctctggacgagcactacaaagttctgacaagacggaacttgtacagcaaaatcatgatttcaaaaagaaactaaataatgtagtgaccttaactaaagaaaggaattttgacaaacttcctttgagcgtatgtatcaacgatggtgctgatagtcacttgaatgaagaatggaatgttcaagttattaagttcttagaaactcttttcccattaccatcatcatttgagaaactcgagcaccaccaccaccaccactga

SEQ ID NO: 22 - protein, 571 aa, Neisseria meningitidis
MASWSHPQFEKGALVPRGSFILNNRKWRKLKRDPSAFFRDSKFNFLRYFSAKKFAKNFKNSSHIHKTNISKAQSNISSTLKQNRKQDMLIPINFFNFEYIVKKLNNQNAIGVYILPSNLTLKPALCILESHKEDFLNKFLLTISSENLKLQYKFNGQIKNPKSVNEIWTDLFSIAHVDMKLSTDRTLSSSISQFWFRLEFCKEDKDFILFPTANRYSRKLWKHSIKNNQLFKEGIRNYSEISSLPYEEDHNFDIDLVFTWVNSEDKNWQELYKKYKPDFNSDATSTSRFLSRDELKFALRSWEMNGSFIRKIFIVSNCAPPAWLDLNNPKIQWVYHEEIMPQSALPTFSSHAIETSLHHIPGISNYFIYSNDDFLLTKPLNKDNFFYSNGIAKLRLEAWGNVNGECTEGEPDYLNGARNANTLLEKEFKKFTTKLHTHSPQSMRTDILFEMEKKYPEEFNRTLHNKFRSLDDIAVTGYLYHHYALLSGRALQSSDKTELVQQNHDFKKKLNNVVTLTKERNFDKLPLSVCINDGADSHLNEEWNVQVIKFLETLFPLPSSFEKLEHHHHHH

SEQ ID NO: 23 - DNA, 1197 bp, Neisseria meningitidis
atggctagctggagccacccgcagttcgaaaaaggcgccctggttccgcgtggatccaaagtcttaaccgtctttggcactcgccctgaagctattaaaatggcgcctgtaattctagagttacaaaaacataacacaattacttcaaaagtttgcattactgcacagcatcgtgaaatgctagatcaggttttgagcctattcgaaatcaaagctgattatgatttaaatatcatgaaacccaaccagagcctacaagaaatcacaacaaatatcatctcaagccttaccgatgttcttgaagatttcaaacctgactgcgtccttgctcacggagacaccacaacaacttttgcagctagccttgctgcattctatcaaaaaatacctgttggccacattgaagcaggcctgagaacttataatttatactctccttggccagaggaagcaaataggcgtttaacaagcgttctaagccagtggcattttgcacctactgaagattctaaaaataacttactatctgaatcaataccttctgacaaagttattgttactggaaatactgtcatagatgcactaatggtatctctagaaaaactaaaaataactacaattaaaaaacaaatggaacaagcttttccatttattcaggacaactctaaagtaattttaattaccgctcatagaagagaaaatcatggggaaggtattaaaaatattggactttctatcttagaattagctaaaaaatacccaacattctcttttgtgattccgctccatttaaatcctaacgttagaaaaccaattcaagatttattatcctctgtgcacaatgttcatcttattgagccacaagaatacttaccattcgtatatttaatgtctaaaagccatataatattaagtgattcaggcggcatacaagaagaagctccatccctaggaaaaccagttcttgtattaagagatactacagaacgtcctgaagctgtagctgcaggaactgtaaaattagtaggttctgaaactcaaaatattattgagagctttacacaactaattgaataccctgaatattatgaaaaaatggctaatattgaaaacccttacgggataggtaatgcctcaaaaatcattgtagaaactttattaaagaatagactcgagcaccaccaccaccaccactga

SEQ ID NO: 24 - protein, 398 aa, Neisseria meningitidis
MASWSHPQFEKGALVPRGSKVLTVFGTRPEAIKMAPVILELQKHNTITSKVCITAQHREMLDQVLSLFEIKADYDLNIMKPNQSLQEITTNIISSLTDVLEDFKPDCVLAHGDTTTTFAASLAAFYQKIPVGHIEAGLRTYNLYSPWPEEANRRLTSVLSQWHFAPTEDSKNNLLSESIPSDKVIVTGNTVIDALMVSLEKLKITTIKKQMEQAFPFIQDNSKVILITAHRRENHGEGIKNIGLSILELAKKYPTFSFVIPLHLNPNVRKPIQDLLSSVHNVHLIEPQEYLPFVYLMSKSHIILSDSGGIQEEAPSLGKPVLVLRDTTERPEAVAAGTVKLVGSETQNIIESFTQLIEYPEYYEKMANIENPYGIGNASKIIVETLLKNRLEHHHHHH

SEQ ID NO: 25 - DNA, 30 bp, artificial sequence
  source: /note=“Description of artificial sequence oligonucleotide”
gcggatccgctgttattatatttgttaacg

SEQ ID NO: 26 - DNA, 30 bp, artificial sequence
  source: /note=“Description of artificial sequence oligonucleotide”
ccgctcgagtttttcttggccaaaaaactg

SEQ ID NO: 27 - DNA, 34 bp, artificial sequence
  source: /note=“Description of artificial sequence oligonucleotide”
gcatctcatatggctgttattatatttgttaacg

SEQ ID NO: 28 - DNA, 32 bp, artificial sequence
  source: /note=“Description of artificial sequence primer”
gcggatccattatgagcaaaattagcaaattg

SEQ ID NO: 29 - DNA, 29 bp, artificial sequence
  source: /note=“Description of artificial sequence primer”
ccgctcgagttgtccactaggctgtgatg

SEQ ID NO: 30 - DNA, 31 bp, artificial sequence
  source: /note=“Description of artificial sequence primer”
ctgactccatatgacgaacccgtccaactcc

SEQ ID NO: 31 - DNA, 34 bp, artificial sequence
  source: /note=“Description of artificial sequence primer”
cttagcggccgcatcaactttgccgggtcagccg

SEQ ID NO: 32 - DNA, 67 bp, artificial sequence
  source: /note=“Description of artificial sequence primer”
cttacatatgcatcatcatcatcatcacgctagcggatccatgacaacgaactatatttttgtgacc

SEQ ID NO: 33 - DNA, 34 bp, artificial sequence
  source: /note=“Description of artificial sequence primer”
cttagcggccgcttacttcgcctgacgtttctgg

SEQ ID NO: 34 - DNA, 33 bp, artificial sequence
  source: /note=“Description of artificial sequence primer”
gcagatcttttatacttaataacagaaaatggc

SEQ ID NO: 35 - DNA, 32 bp, artificial sequence
  source: /note=“Description of artificial sequence primer”
ccgctcgagtttctcaaatgatgatggtaatg

SEQ ID NO: 36 - DNA, 29 bp, artificial sequence
  source: /note=“Description of artificial sequence primer”
gcggatccaaagtcttaaccgtctttggc

SEQ ID NO: 37 - DNA, 34 bp, artificial sequence
  source: /note=“Description of artificial sequence primer”
ccgctcgagtctattctttaataaagtttctaca

(19) United States
(12) Patent Application Publication
(10) Pub. No.: US 2013/0012471 A1
(43) Pub. Date: Jan. 10, 2013

(54) MEANS AND METHODS FOR PRODUCING ARTIFICIAL CAPSULAR POLYSACCHARIDES OF NEISSERIA MENINGITIDIS

(76) Inventors: Rita Gerardy-Schahn, Hiddenhausen (DE); Martina Mühlanhoff, Hannover (DE); Andrea Bethe, Hildesheim (DE); Katharina Stummeyer, Konigsointer (DE); Friedrich Freiberger, Hannover (DE); Sebastian Damerow, Dundee (GB)

(21) Appl. No.: 13/390,424

(22) PCT Filed: Aug. 26, 2010
(86) PCT No.: PCT/EP10/62481

§ 371 Date: Sep. 28, 2012

(30) Foreign Application Priority Data
Aug. 26, 2009 (EP) 09168765.7

(51) Int. Cl. A61K 31/715; C12P 19/30; C07H 21/04; C12N 15/63; C12N 1/15; C13K 13/00; A61P 31/04; C12N 1/21; C12N 1/19; C12P 19/04; C12N 9/14

(52) U.S. Cl. 514/54; 435/101; 435/89; 536/23.2; 435/320.1; 435/252.33; 435/195; 536/123.1; 435/252.3; 435/254.11; 435/254.2

(57) ABSTRACT

The invention provides for an in vitro method for producing capsular polysaccharides of Neisseria meningitidis. The invention also provides capsular polysaccharides obtainable by the methods described herein. The capsular polysaccharides comprise capsular polysaccharide specific for Neisseria meningitidis serogroups W-135, Y, X and A. Also encompassed are chimeric capsular polysaccharides comprising or composed of CPS of Neisseria meningitidis serogroups Y/W-135, W-135/Y, B/Y, C/Y, B/W-135, C/W-135, B/Y/W-135, C/Y/W-135, B/W-135/Y, C/W-135/Y. X/A or A/X. The invention also provides for the use of these capsular polysaccharides foi as pharmaceuticals, particularly as vaccines and/or diagnostics.

The present invention relates to means and methods for producing synthetic and artificial capsular polysaccharides of Neisseria meningitidis. The present invention also relates to capsular polysaccharides obtainable by the inventive method. Also provided are capsular polysaccharides of Neisseria meningitidis for use as pharmaceuticals, particularly as vaccines and/or diagnostics.

Bacterial meningitidis remains a serious threat to global health, accounting for an estimated annual 170,000 deaths worldwide (WHO, http://www.who.int/nuvi/meningitidis/en/). Despite the availability of potent antimicrobial agents, case-fatality rates are high (10-40%) and survivors frequently suffer from sequeae such as neurologic disability or limb loss and deafness (Van Deuren et al. Clin Microbiol Rev 2000; 13(1): 144-166; Kaper et al., Nat Rev Microbiol 2004, 2(2): 123-140). Neisseria meningitidis (Nm) is one of the most important causative agents of bacterial meningitidis because of its potential to spread in epidemic waves (Kaper et al., Nat Rev Microbiol 2004, 2(2): 123-140; Rosenstein et al., N Eng J Med 2001, 344(18): 1378-1388). Crucial virulence determinants of disease causing Nm species are their extracellular polysaccharide capsules that are essential for meningococcal survival in human serum (Vogel et al., Infect Immun 1997, 65(10): 4022-4029). Based on antigenic variation of these polysaccharides at least twelve different serogroups of Nm have been identified (A, B, C, E29, H, I, K, L, W-135, X, Y and Z), but only six (A, B, C, W-135, Y and X) account for virtually all cases of disease; see also Frosch, M., VOGEL, U. (2006) “Structure and genetics of the meningococcal capsule.” In Handbook of Meningococcal Disease. Frosch, M., Maiden, M. C. J. (eds). Weinheim: Wiley-VCH.

Serogroup A (NmA) and C (NmC) are the main causes of meningococcal meningitidis in sub-Saharan Africa, while serogroups B (NmB) and C are the major disease causing isolates in industrialized countries. However, serogroups W-135 (NmW-135) and Y (NmY) are becoming increasingly prevalent. For NmW-135, this is most explicitly evidenced by the 2002 epidemic in Burkina Faso with over 13,000 cases and more than 1,400 deaths (Connolly et al., Lancet 2004, 364(9449): 1974-1983; WHO, Epidemic and Pandemic Alert and Response (EPR) 2008). In contrast, NmY is gaining importance in the United States where its prevalence increased from 2% during 1989-1991 to 37% during 1997-2002 (Pollard et al., J Paediatr Child Health 2001, 37(5): S20-S27). Recently, also the previously only sporadically found serogroup X (NmX) appeared with high incidence in Niger and caused outbreaks in Kenya and Uganda (Biosier et al., Clin Infect Dis 2007, 44(5): 657-663; Lewis, WHO Health Action in Crisis 1, 6 2006).

The serogroups A, B, C, 29E, H, I, K, L, W-135, X, Y and Z are well known in the art and are described in Frosch, M., VOGEL, U. (2006) loc. cit. The capsular polysaccharides (CPS) of all serogroups are negatively charged linear polymers. Serogroup B and C are encapsuled in homopolymeric CPS composed of sialic acid (Neu5Ac) moieties that are linked by either α-2→8 glycosidic linkages in serogroup B or by α-2→9 linkages in serogroup C (Bhattacharjee et al., J Biol Chem 1975, 250(5): 1926-1932). Serogroup W-135 and Y both are heteropolymers. They are composed of either galactose/Neu5Ac repeating units [→6)-α-D-Glcp-(1→4)-α-Neu5Ac-(2→]ₙ in serogroup W-135 or glucose/Neu5Ac repeating units [→6)-α-D-Galp-(1→4)-α-Neu5Ac-(2→]ₙ in serogroup Y (Bhattacharjee et al., Can J Biochem 1976, 54(1): 1-8). The CPS of NmA and NmX do not contain Neu5Ac moieties, but are instead built from N-Acetyl-mannosamine 1-phosphate [→6)-α-D-ManpNAc-(1→OPO₃→]ₙ or N-Acetyl-glucosamine 1-phosphate [→6)-α-D-GlcpNAc-(1→OPO₃→]ₙ repeating units, respectively (Bundle et al., Carbohydr Res 1973, 26(1): 268-270; Bundle et al., J Biol Chem 1974, 249(15): 4797-4801); Bundle et al., J Biol Chem 1974, 249(7): 2275-2281; Jennings et al., J Infect Dis 1977, 136 Suppl: S78-S83).

The CPS of disease causing Nm are attractive vaccine candidates and polysaccharide or polysaccharide-conjugate vaccines are available for serogroups A, C, Y, W-135 (Broker et al., Minerva Med 2007, 98(5):575-589). Currently no vaccines are available for serogroups B and X. The capsular polysaccharide of serogroup B is only poorly immunogenic, because it is structurally and chemically identical to a polycarbohydrate found in humans (polySia). Major outbreaks of NmX, however, occurred only in 2006 wherefore no vaccine has been developed yet.

Key enzymes in the CPS biosynthesis are membrane associated capsule polymerases. Candidate genes have been identified for all six disease causing serogroups (Frosch et al., Proc Natl Acad Sci USA 1989, 86(5): 1669-1673; Claus et al., Mol Gen Genet. 1997, 257(1): 28-34; Tzeng et al., Infect Immun 2003, 71(2): 6712-6720). However, our knowledge of enzymology or structure-function relations of those important enzymes is still very limited. Though some data had been reported for the NmB and NmC enzymes using crude membrane fractions as enzyme source (Steenbergen et al., J Biol Chem 2003, 278(17): 15349-15359), only recently active NmB polymerase could be purified and initial structure-function analyses performed (Freiberger et al., Mol Microbiol 2007, 65(5): 1258-1275). In a most recent study also the purification and initial characterization of the capsule polymerases cloned from serogroups NmW-135 and NmY have been performed (Claus et al., Mol Microbiol 2009, 71(4): 960-971). These proteins are bifunctional glycosyltransferases that are individually able to synthesize the respective heteropolymeric CPS of NmW-135 and NmY.

However, until now polysaccharide production for neisserial vaccines still requires fermentation of Neisseria meningitidis with subsequent multistep purification of the polysaccharides from the culture medium. These production processes are both cost intensive and always at risk for contaminations by neisserial toxins, media components or chemicals required for subsequent purification procedures. Moreover, the obtained polysaccharide batches are often heterogeneous and difficult to characterize.

These technical problems have been overcome by the method of the present invention for producing synthetic and artificial capsular polysaccharides of Neisseria meningitidis in vitro as will be detailed below.

The present invention provides an in vitro method for producing capsular polysaccharides (CPS) of Neisseria meningitidis, said method comprising the steps:
(a) contacting at least one donor carbohydrate with at least one purified capsule polymerase (CP);
(b) incubation of said carbohydrate with said capsular polymerases; and
(c) isolating the resulting capsular polysaccharide,
   wherein the obtained capsular polysaccharides are synthetic or artificial capsular polysaccharides of Neisseria meningitidis serogroup W-135, Y, A, or X specific capsular polysaccharides or wherein the obtained capsular polysaccharides are artificial chimeric capsular polysaccharides comprising capsular polysaccharides or capsular polysaccharide subunits of Neisseria meningitidis serogroups Y/W-135, W-135/Y, B/Y, C/Y, B/W-135, C/W-135, B/Y/W-135, C/Y/W-135, B/W-135/Y, C/W-135/Y, X/A or A/X.

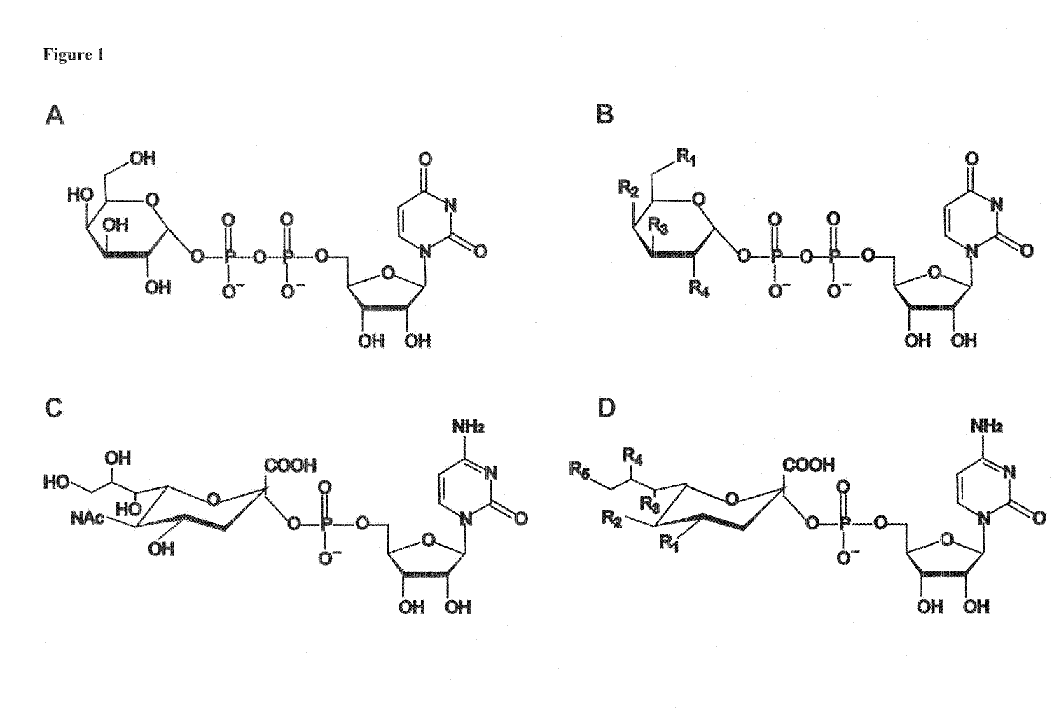

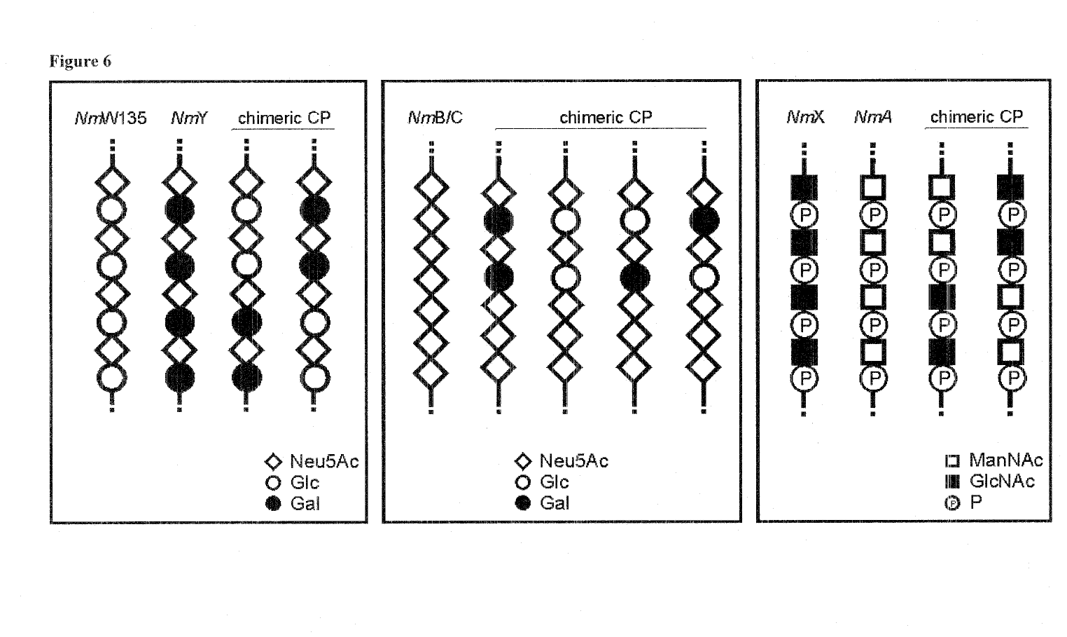

In accordance with the present invention, it was surprisingly found that capsular polysaccharides (CPS) of Neisseria meningitidis serogroups W-135, Y, A, can be synthetically produced, i.e. in vitro. Thereby, the previously used cost- and time-intensive production processes can be avoided. Furthermore, it was found that artificial chimeric CPS comprising CPS or subunits thereof of different Neisseria meningitidis serogroups can be produced by the in vitro method described and exemplified herein. The chimeric CPS obtainable by the herein described in vitro method may comprise or be composed of two or more CPS-subunits of Neisseria meningitidis serogroups A, B, C, W-135, X and/or Y or a CPS which comprises one or more derivatized building blocks of different CPS of Neisseria meningitidis serogroups A, B, C, W-135, X and/or Y. Examples for such derivatized building blocks are shown in FIGS. 1 to 5. The chimeric CPS obtainable by the herein described method may comprise or be composed of CPS or CPS-subunits of Neisseria meningitidis serogroups Y/W-135, W-135/Y, B/Y, C/Y, B/W-135, C/W-135, B/Y/W-135, C/Y/W-135, B/W-135/Y, C/W-135/Y, X/A or A/X. Within said chimeric CPS, one or more building blocks of the CPS-subunits may be derivatized as exemplarily shown in FIGS. 1 to 5. The chimeric CPS obtainable by the inventive method presented hereinabove may contain one or more carbohydrates of each contained CPS-subunit. The sequence of the CPS-subunits of a chimeric CPS obtainable by the herein described method or the derivatized building blocks contained in these chimeric CPS may be of any order. Examples for chimeric CPS obtainable by the in vitro method presented hereinabove are illustrated in FIG. 6.

The chimeric CPS obtainable by the in vitro method described hereinabove are also useful as pharmaceuticals, e.g., as vaccines. In particular, the herein described chimeric CPS are advantageous as vaccines in the prophylaxis and treatment of diseases caused by Neisseria meningitidis, such as neisserial meningitidis. The chimeric CPS obtainable by the herein described in vitro method can be used as vaccines against different Neisseria meningitidis serogroups. These chimeric CPS containing different CPS-subunits can be used against the Neisseria meningitidis serogroups whose CPS-subunits are contained in said chimeric CPS. For example, in accordance with the present invention, a chimeric CPS containing a CPS-subunit of Neisseria meningitidis serogroup A and a CPS-subunit of Neisseria meningitidis serogroup X may be used as a vaccine against both, Neisseria meningitidis serogroup A and Neisseria meningitidis serogroup X. Also multimeric chimeric CPS are obtainable by the present in vitro method. Such a chimeric CPS can contain or be composed of two, three or more different CPS-subunits of different Neisseria meningitidis serogroups. For example, a chimeric CPS obtainable by the herein presented in vitro method can contain or be composed of CPS-subunits of Neisseria meningitidis serogroups W-135, Y and C. Moreover, such chimeric CPS as well as antibodies directed thereto are useful for diagnostic purposes.

In one embodiment of the herein described and exemplified in vitro method, the artificial chimeric CPS comprises CPS of Neisseria meningitidis serogroups W-135 and Y.

In a further embodiment of the herein presented method, the at least one donor carbohydrate and the at least one capsular polymerase (CP) are further contacted with an acceptor carbohydrate.

According to the inventive in vitro method, the donor carbohydrate which is contacted with at least one purified capsule polymerase (CP) may further be activated during step (b). Preferably, the activation is mediated by linkage of an activating nucleotide such as CMP, UDP, TDP or AMP. Most preferably, the activating nucleotide is CMP or UDP. The activation of a carbohydrate by linkage of a nucleotide may be catalysed by several activating enzymes which are known in the art. Such activating enzymes may be contacted with the at least one donor carbohydrate and the at least one CP during step (a) of the in vitro method provided herein. For example, the UDP-sugar pyrophosphorylase (USP) of Leishmania major (USP-LM) is contacted with the at least one donor carbohydrate with the at least one CP during step (a) of the in vitro method presented herein. USP-LM catalyses the activation of both, Gal-1-phosphate and Glc-1-phosphate, to the nucleotide sugars UDP-Gal and UDP-Glc, respectively. The nucleotide sequence of USP-LM is shown in SEQ ID NO: 9. The polypeptide sequence of USP-LM is shown in SEQ ID NO: 10. For the activation of Neu5Ac, CMP-NeuNAc synthetase (CSS) is preferably used (Ganguli et al., J Bacteriol (1994), 176(15): 4583-4589). UDP-ManNAc is preferably synthesized from UDP-GlcNAc using the enzyme UDP-GlcNAc-epimerase. In SEQ ID NO: 11, the nucleotide sequence of UDP-GlcNAc-epimerase cloned from Neisseria meningitidis serogroup A is shown, the corresponding polypeptide sequence of UDP-GlcNAc-epimerase is shown in SEQ ID NO: 12.

According to the herein presented method, the at least donor carbohydrate and the capsule polymerase (CP) may be further contacted with an acceptor carbohydrate.

In one embodiment of the herein presented in vitro method, the capsule polymerase (CP) which is contacted with at least one donor carbohydrate is specific for synthesis of the CPS of Neisseria meningitidis serogroup W-135. Specifically, the CP contacted with at least one donor carbohydrate is CP-W-135 or a functional derivative thereof. The nucleotide sequence encoding CP-W-135 is shown in SEQ ID NO: 1. The amino acid sequence of CP-W-135 is shown in SEQ ID NO: 2. A functional derivative of CP-W-135 is an enzyme which is capable of synthesizing capsular polysaccharide (CPS) of serogroup W-135 and of serogroup Y CPS (Claus et al., Mol Microbiol 2009, 71(4): 960-971). Preferably, the nucleotide sequence of a functional derivative of CP-W-135 has a sequence identity to SEQ ID NO: 1 of at least 80%, more preferably at least 85%, more preferably at least 90%, and most preferably at least 95% and the amino acid sequence of a functional derivative of CP-W-135 has a sequence identity to SEQ ID NO: 2 of at least 80%, more preferably at least 85%, more preferably at least 90%, more preferably at least 95%, and most preferably at least 99%. A functional derivative may also comprise a functional fragment maintaining the biological activity. Therefore, the term “functional derivative thereof” as used herein in context of nucleotide sequences or polypeptides refers to a functional fragment which has essentially the same (biological) activity as the nucleotide sequences or polypeptides defined herein (e.g. as shown in SEQ ID NO: 2) which may be encoded by the nucleic acid sequence of the present invention (e.g. SEQ ID NO: 1). The (biological) function can, inter alia, be assessed by the method described in Claus et al., Mol Microbiol 2009, 71(4): 960-971 as well as in the invention provided herein.

According to the present invention, identity levels of nucleotide or amino acid sequences refer to the entire length of nucleotide sequence of SEQ ID NO: 1 or polypeptide sequence of SEQ ID NO: 2, respectively and is assessed pair-wise, wherein each gap is to be counted as one mismatch. The term “identity” as used herein is used equivalently to the term “homology”.

For example, the terms identity and homology are used herein in the context of a nucleic acid or a polypeptide/amino acid sequence which has an identity or homology or at least 80% to SEQ ID NO: 1 or 2, respectively, preferably over the entire length.

Accordingly, the present invention relates to the use of a polypeptide (being a CP-W-135 or fragment thereof) in the present inventive method, wherein the polypeptide has at least 80% identity/homology to the polypeptide shown in SEQ ID NO: 2.

If, e.g., two nucleic acid sequences to be compared by, e.g., sequence comparisons differ in identity, then the term “identity” or “homology” refers to the shorter sequence and that part of the longer sequence that matches said shorter sequence. Therefore, when the sequences which are compared do not have the same length, the degree of identity preferably either refers to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that “matches” the shorter sequence. Also, these definitions for sequence comparisons (e.g., establishment of “identity” or “homology” values) are to be applied for all sequences described and disclosed herein.

Identity, moreover, means that there is a functional and/or structural equivalence between the corresponding nucleotide sequence or polypeptides, respectively (e.g., polypeptides encoded thereby). Nucleic acid/amino acid sequences having the given identity levels to the herein-described particular nucleic acid/amino acid sequences may represent derivatives/variants of these sequences which, preferably, have the same biological function. They may be either naturally occurring variations, for instance sequences from other varieties, species, etc., or mutations, and said mutations may have formed naturally or may have been produced by deliberate mutagenesis. Furthermore, the variations may be synthetically produced sequences. The allelic variants may be naturally occurring variants or synthetically produced variants or variants produced by recombinant DNA techniques. Deviations from the above-described nucleic acid sequences may have been produced, e.g., by deletion, substitution, addition, insertion and/or recombination. The term “addition” refers to adding at least one nucleic acid residue/amino acid to the end of the given sequence, whereas “insertion” refers to inserting at least one nucleic acid residue/amino acid within a given sequence. The term “deletion” refers to deleting or removal at least one nucleic acid residue/amino acid residue in a given sequence. The term “substitution” refers to the replacement of at least one nucleic acid residue/amino acid residue in a given sequence. Again, these definitions as used here apply, mutatis mutandis, for all sequences provided and described herein.

Variant polypeptides and, in particular, the polypeptides encoded by the different variants of the nuclei acid sequences to be used in accordance with the inventive in vitro method described herein preferably exhibit certain characteristics they have in common. These include, for instance, biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

In a further embodiment of the hereinabove described in vitro method, the capsular polymerase (CP) is CP-W-135 or a functional derivative thereof and at least one donor carbohydrate which is contacted with the CP is CMP-Neu5Ac or a derivative thereof and at least one donor carbohydrate is UDP-Gal or a derivative thereof. Examples for derivatives of CMP-Neu5Ac and UDP-Gal are illustrated in FIGS. 1D and 1B, respectively.

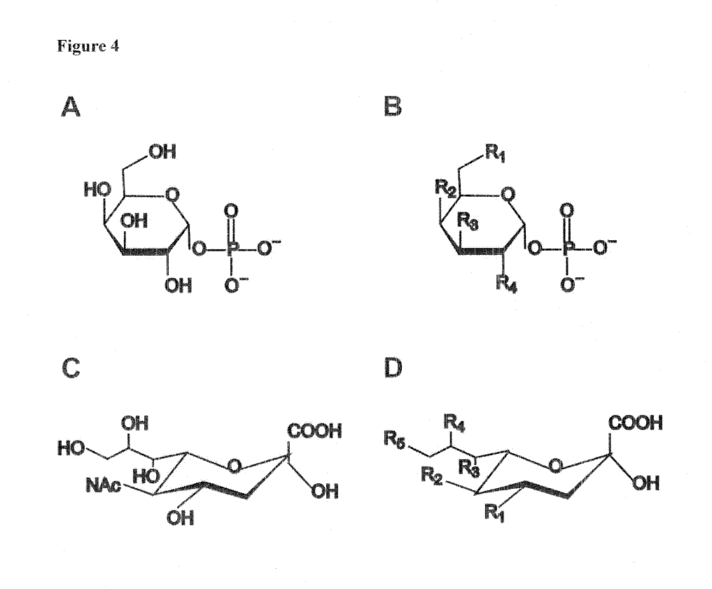

In another embodiment of the in vitro method presented herein, the CP is CP-W-135 or a functional derivative thereof and at least one donor carbohydrate is Gal-1-phosphate or a derivative thereof and at least one donor carbohydrate is sialic acid or a derivative thereof. Examples for derivatives of Gal-1-phosphate and sialic acid are illustrated in FIGS. 4B and 4D, respectively. Preferably, in accordance with the herein described in vitro method, the sialic acid is Neu5Ac. In the hereinabove described in vitro method, the Gal-1-phosphate and sialic acid may be further contacted with at least one nucleotide and/or phosphoenolpyruvate (PEP) and auxiliary enzymes when contacted with the CP. Such a nucleotide can be CMP, CDP, CTP, UMP, UDP and UTP. At least one of the donor carbohydrates Gal-1-phosphate and sialic acid may be activated during incubation with the CP in the in vitro method presented herein to yield the activated sugar nucleotides UDP-Gal and/or CMP-Neu5Ac.

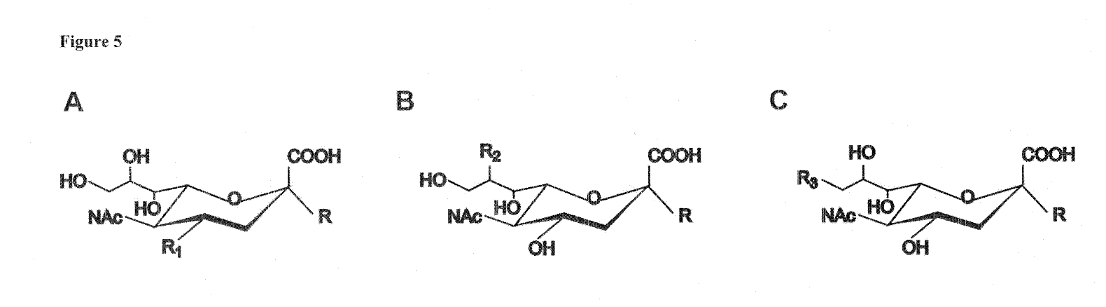

In accordance with the hereinabove described and exemplified in vitro method, CP-W-135 or a functional derivative thereof and the at least one donor carbohydrate may further be contacted with an acceptor carbohydrate during the contacting step. Said acceptor carbohydrate may be oligomeric or polymeric CPS of Neisseria meningitidis serogroup W-135 (W-135 CPS), oligomeric or polymeric CPS of Neisseria meningitidis serogroup Y (Y CPS), oligomeric or polymeric CPS of Neisseria meningitidis serogroup B (B CPS; α2,8-linked sialic acid) and/or oligomeric or polymeric CPS of Neisseria meningitidis serogroup C (C CPS; α-2,9-linked sialic acid). Said acceptor carbohydrate may also carry one or more additional functional groups at its reducing end as exemplified in the legend of FIG. 5. Accordingly, an artificial chimeric CPS obtainable by the in vitro method described herein comprising or composed of CPS or CPS-subunits of Neisseria meningitidis serogroups Y/W-135, W-135/Y, B/Y, C/Y, B/W-135, C/W=135, B/Y/W-135, C/Y/W-135, B/W-135/Y or C/W-135/Y can be synthesized. For Example, in the in vitro method of the present invention, CP-W-135 is contacted with CMP-Neu5Ac and UDP-Gal as donor carbohydrates and trimeric α2,8-linked sialic acid (trimeric B CPS) as an acceptor carbohydrate to synthesize an artificial chimeric CPS comprising or composed of subunits of CPS of Neisseria meningitidis serogroups B/W-135.

Within a chimeric CPS obtainable by the inventive method described hereinabove, one or more carbohydrates of the CPS-subunits may be derivatized and may contain, for example, additional functional groups such as amino groups, alkyl groups, hydroxyl groups, carboxylic acids, azides, amides, acetyl groups or halogen atoms; see also “Carbohydrate chemistry” Volumes 1-34: monosaccharides, disaccharides, and specific oligosaccharides, Reviews of the literature published during 1967-2000, Cambridge (England), Royal Society of Chemistry. The chimeric CPS obtainable by the in vitro method presented herein may contain one or more carbohydrates of each contained CPS-subunit. The sequence of the CPS-subunits of said chimeric CPS may be of any order.

As an example, the in vitro method for producing capsular polysaccharides (CPS) of Neisseria meningitidis comprises the steps:
(a) contacting CMP-Neu5Ac, UDP-Gal and hydrolysed Y CPS with CP-W-135;
(b) incubation of CMP-Neu5Ac, UDP-Gal and hydrolysed Y CPS with CP-W-135; and
(c) isolating the artificial chimeric CPS composed of capsular polysaccharide subunits of Neisseria meningitidis serogroups Y/W-135.

The skilled person readily understands that also other combinations of activated or non-activated donor carboh_{y} drates, acceptor carbohydrates and capsule polymerases (CP) as described herein can be applied. Such other combinations and other modifications do not defer from the gist of the present invention.

For example, another exemplifying in vitro method of the present invention relates to a method for producing capsular polysaccharides (CPS) of Neisseria meningitidis comprises the steps:
(a) contacting Neu5Ac, Gal-1-P, CTP, UTP, and hydrolysed Y CPS with CP-W-135, USP-LM and CSS;
(b) incubation of Neu5Ac, Gal-1-P, CTP, UTP, and hydrolysed Y CPS with CP-W-135, USP-LM and CSS wherein Neu5Ac is activated to CMP-Neu5Ac and Glc-1-P is activated to UDP-Glc; and
(c) isolating the artificial chimeric CPS composed of capsular polysaccharide subunits of Neisseria meningitidis serogroups Y/W-135.

The skilled person readily understands that also other combinations of activated or non-activated donor carbohydrates, acceptor carbohydrates and capsule polymerases (CP) as described herein can be applied. Such other combinations and other modifications do not defer from the gist of the present invention.

In another embodiment of the in vitro method presented herein, the capsular polymerase (CP) which is contacted with at least one donor carbohydrate is specific for synthesis of the CPS of Neisseria meningitidis serogroup Y. Specifically, the CP contacted with at least one donor carbohydrate is CP-Y or a functional derivative thereof. The nucleotide sequence encoding CP-Y is shown in SEQ ID NO: 3. The amino acid sequence of CP-Y is shown in SEQ ID NO: 4. A functional derivative of CP-Y is an enzyme which is capable of synthesizing capsular polysaccharide of serogroup W-135 and of serogroup Y CPS (Claus et al., Mol Microbiol 2009, 71(4): 960-971). Preferably, the nucleotide sequence of a functional derivative of CP-Y has a sequence identity to SEQ ID NO: 3 of at least 40%, at least 80%, more preferably at least 85%, more preferably at least 90%, and most preferably at least 95% and the amino acid sequence of a functional derivative of CP-Y has a sequence identity to SEQ ID NO: 4 of at least 80%, more preferably at least 85%, more preferably at least 90%, more preferably at least 95%, and most preferably at least 99%. A functional derivative may also comprise a functional fragment maintaining the biological activity. Therefore, the term “functional derivative thereof” as used herein in context of nucleotide sequences or polypeptides refers to a functional fragment which has essentially the same (biological) activity as the nucleotide sequences or polypeptides defined herein (e.g. as shown in SEQ ID NO: 4) which may be encoded by the nucleic acid sequence of the present invention (e.g. SEQ ID NO: 3). The (biological) function can, inter alia, be assessed by the method described in Claus et al., Mol Microbiol 2009, 71(4): 960-971 as well by methods provided herein.

According to the present invention, identity levels of nucleotide or amino acid sequences refer to the entire length of nucleotide sequence of SEQ ID NO: 3 or polypeptide sequence of SEQ ID NO: 4, respectively and is assessed pair-wise, wherein each gap is to be counted as one mismatch. The term “identity” as used herein is used equivalently to the term “homology”. For example, the terms identity and homology are used herein in the context of a nucleic acid or a polypeptide/amino acid sequence which has an identity or homology of at least 80% to SEQ ID NO: 3 or 4, respectively, preferably over the entire length.

Accordingly, the present invention relates to the use of a polypeptide (being a CP-Y or fragment thereof) in the present inventive method, wherein the polypeptide has at least 80% identity/homology to the polypeptide shown in SEQ ID NO: 4.

If, e.g., two nucleic acid sequences to be compared by, e.g., sequence comparisons differ in identity, then the term “identity” or “homology” refers to the shorter sequence and that part of the longer sequence that matches said shorter sequence. Therefore, when the sequences which are compared do not have the same length, the degree of identity preferably either refers to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that “matches” the shorter sequence. Also, these definitions for sequence comparisons (e.g., establishment of “identity” or “homology” values) are to be applied for all sequences described and disclosed herein. The terms “identity” and “homology” were further characterized hereinabove and the definitions and explanations apply, mutatis mutandis, for CP-Y and functional fragments thereof.

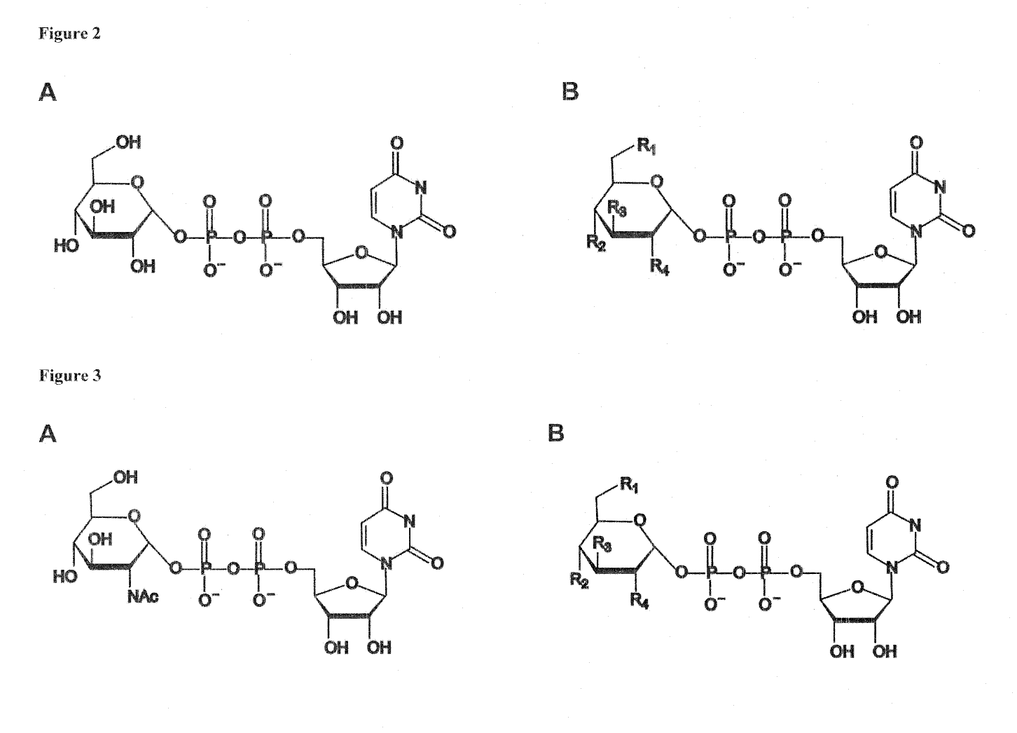

In a specific embodiment of the inventive in vitro method, the CP is CP-Y or a functional derivative thereof and at least one donor carbohydrate is CMP-Neu5Ac or a derivative thereof and at least one donor carbohydrate is UDP-Glc or a derivative thereof. Examples for derivatives of CMP-Neu5Ac and UDP-Glc are illustrated in FIGS. 1D and 2B, respectively. Again, the term derivatives or functional fragments in accordance with the invention relates to derivatives or fragments that are biologically active. Such a “biological” function may be tested in assays as provided in the appended examples or as described in Claus (2009), loc crit.

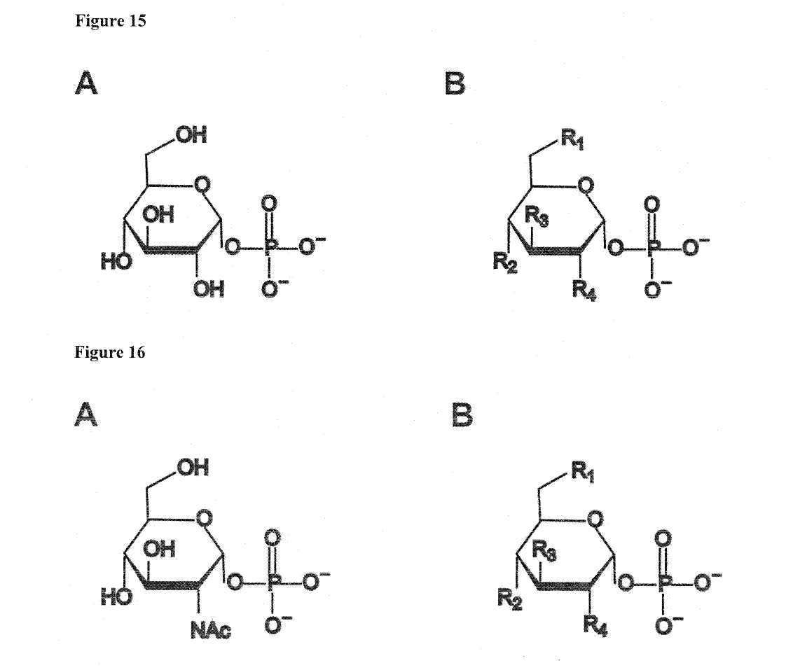

In a further embodiment of the herein presented in vitro method, the capsular polymerase (CP) is CP-Y or a functional derivative thereof and at least one donor carbohydrate is Glc-1-phosphate or a derivative thereof and at least one donor carbohydrate is sialic acid or a derivative thereof. Examples for derivatives of sialic acid are illustrated in FIG. 4D, examples for derivatives of Glc-1-phosphate are illustrated in FIG. 15. In a preferred embodiment, said sialic acid is Neu5Ac. In accordance with the herein described in vitro method, the Glc-1-phosphate and sialic acid may be further contacted with at least one nucleotide and/or phosphoenolpyruvate (PEP) and auxiliary enzymes when contacted with the CP. Such a nucleotide can be CMP, CDP, CTP, UMP, UDP and UTP. At least one of the donor carbohydrates Glc-1-phosphate and sialic acid may be activated during incubation with the CP in the in vitro method presented herein to yield the activated sugar nucleotides UDP-Glc and/or CMP-Neu5Ac.

CP-Y or a functional derivative thereof and the at least one donor carbohydrate may further be contacted with an acceptor carbohydrate during the contacting step of the herein presented in vitro method. Said acceptor carbohydrate may be oligomeric or polymeric W-135 CPS, oligomeric or polymeric Y CPS, oligomeric or polymeric B CPS and/or oligomeric or polymeric C CPS. Said acceptor carbohydrate may also carry one or more additional functional groups at its reducing end (See and legend 5). Accordingly, a chimeric CPS obtainable by the in vitro method of the present invention comprising or composed of CPS or CPS-subunits of Neisseria meningitidis serogroups Y/W-135, W-135/Y, B/Y, C/Y, B/W-135, C/W-135, B/Y/W-135, C/Y/W-135, B/W-135/Y or C/W-135/Y can be synthesized. For example, CP-Y is contacted with donor carbohydrates CMP-Neu5Ac and UDP-Glc and with oligomeric W-135 CPS as an acceptor to synthesize an artificial chimeric CPS comprising or composed of subunits of CPS of Neisseria meningitidis serogroups W-135/Y. Also in this context, the term “functional derivative” may also comprise “functional fragments”.

Within a chimeric CPS obtainable by the in vitro method presented herein, one or more carbohydrates of the CPS-subunits may be derivatized and may contain, for example, additional functional groups such as amino groups, alkyl groups, hydroxyl groups, carboxylic acids, azides, amides, acetyl groups or halogen atoms; see, e.g., “Carbohydrate chemistry” Volumes 1-34 Cambridge [England], Royal Society of Chemistry, loc. cit. Said chimeric CPS may contain one or more carbohydrates of each contained CPS-subunit. The sequence of the CPS-subunits of the chimeric CPS obtainable by the herein described in vitro method may be of any order.

As an example of the present invention, the in vitro method for producing capsular polysaccharides (CPS) of Neisseria meningitidis may comprise the steps:
(a) contacting CMP-Neu5Ac, UDP-Glc and hydrolysed W-135 CPS with CP-Y;
(b) incubation of CMP-Neu5Ac, UDP-Glc and hydrolysed W-135 CPS with CP-Y; and
(c) isolating the artificial chimeric CPS composed of capsular polysaccharide subunits of Neisseria meningitidis serogroups W-135/Y.

As mentioned above, the skilled person readily understands that also other combinations of activated or non-activated donor carbohydrates, acceptor carbohydrates and capsule polymerases (CP) as described herein can be applied. Such other combinations and other modifications do not defer from the gist of the present invention.

Another exemplifying in vitro method of the present invention relates to a method for producing capsular polysaccharides (CPS) of Neisseria meningitidis comprises the steps:
(a) contacting Neu5Ac, Glc-1-P, CTP, UTP and hydrolysed W-135 CPS with CP-Y, USP-LM and CSS;
(b) incubation of Neu5Ac, Glc-1-P, CDP, UDP, PEP and hydrolysed W-135 CPS with CP-Y, USP-LM and CSS, wherein Neu5Ac is activated to CMP-Neu5Ac and Glc-1-P is activated to UDP-Glc; and
(c) isolating the artificial chimeric CPS composed of capsular polysaccharide subunits of Neisseria meningitidis serogroups Y/W-135.

Again, also other combinations of activated or non-activated donor carbohydrates, acceptor carbohydrates and capsule polymerases (CP) as described herein can be applied. Such other combinations and other modifications do not defer from the gist of the present invention.

The present invention also relates to an in vitro method wherein the capsular polymerase (CP) which is contacted with at least one donor carbohydrate is specific for synthesis of the CPS of Neisseria meningitidis serogroup X. Specifically, the CP contacted with at least one donor carbohydrate is CP-X or a functional derivative thereof. The nucleotide sequence encoding CP-X is shown in SEQ ID NO: 5. The amino acid sequence of CP-X is shown in SEQ ID NO: 6. A functional derivative of CP-X is an enzyme which is capable of synthesizing capsular polysaccharide of serogroup X (Tzeng et al., Infect Immun 2003, 71(2): 6712-6720). Preferably, the nucleotide sequence of a functional derivative of CP-X has a sequence identity to SEQ ID NO: 5 of at least 80%, more preferably at least 85%, more preferably at least 90%, and most preferably at least 95% and the amino acid sequence of a functional derivative of CP-X has a sequence identity to SEQ ID NO: 6 of at least 80%, more preferably at least 85%, more preferably at least 90%, more preferably at least 95%, and most preferably at least 99%. A functional derivative may also comprise a functional fragment maintaining the biological activity. Therefore, the term “functional derivative thereof” as used herein in context of nucleotide sequences or polypeptides refers to a functional fragment which has essentially the same (biological) activity as the nucleotide sequences or polypeptides defined herein (e.g. as shown in SEQ ID NO: 6) which may be encoded by the nucleic acid sequence of the present invention (e.g. SEQ ID NO: 5). Again, also functional fragments are comprised in the term “functional derivative”. The (biological) function can, inter alia, be assessed by the method described in Tzeng et al., Infect Immun 2003, 71(2): 6712-6720 as well as in the methods provided herein.

According to the present invention, identity levels of nucleotide or amino acid sequences refer to the entire length of nucleotide sequence of SEQ ID NO: 5 or polypeptide sequence of SEQ ID NO: 6, respectively and is assessed pair-wise, wherein each gap is to be counted as one mismatch. The term “identity” as used herein is used equivalently to the term “homology”.

For example, the terms identity and homology are used herein in the context of a nucleic acid or a polypeptide/amino acid sequence which has an identity or homology of at least 80% to SEQ ID NO: 5 or 6, respectively, preferably over the entire length.

Accordingly, the present invention relates to the use of a polypeptide (being a CP-X or fragment thereof) in the present inventive method, wherein the polypeptide has at least 80% identity/homology to the polypeptide shown in SEQ ID NO: 6.

If, e.g., two nucleic acid sequences to be compared by, e.g., sequence comparisons differ in identity, then the term “identity” or “homology” refers to the shorter sequence and that part of the longer sequence that matches said shorter sequence. Therefore, when the sequences which are compared do not have the same length, the degree of identity preferably either refers to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that “matches” the shorter sequence. Also, these definitions for sequence comparisons (e.g., establishment of “identity” or “homology” values) are to be applied for all sequences described and disclosed herein. Again, the terms “identity” and “homology” were further characterized hereinabove and the definitions and explanations apply, mutatis mutandis, for CP-X and functional fragments thereof.

The CP to be applied in the means and methods described herein may be CP-X or a functional derivative thereof and at least one donor carbohydrate may be UDP-GlcNAc or a derivative thereof. Examples for derivatives of UDP-GlcNAc may be compounds that are alkylated or hydroxylated or that comprise additional functional groups, such as carboxylic acids, azides, amides, acetyl groups or halogen atoms as also illustrated in FIG. 3B; see also “Carbohydrate chemistry” Volumes 1-34, Cambridge [England], Royal Society of Chemistry, loc. cit.

In another embodiment of the inventive in vitro method, the capsular polymerase (CP) may be CP-X or a functional derivative thereof and at least one donor carbohydrate may be GlcNAc-1-phosphate or a derivative thereof. Examples for derivatives of GlcNAc-1-phosphate are illustrated in FIG. 16. Said donor carbohydrate GlcNAc-1-phosphate may be further contacted with at least one nucleotide and/or phosphoenolpyruvate (PEP) and auxiliary enzymes when contacted with the CP. Said nucleotide can be UMP, UDP and UTP. Said donor carbohydrate GlcNAc-1-phosphate may further be activated during incubation with the CP. In accordance with the herein presented in vitro method, this activation may yield the activated sugar nucleotide UDP-GlcNAc.

Generally, in context with the present invention, derivatives of the saccharides described herein may also be labelled forms of these saccharides. For example, for derivatives of the saccharides described herein, the saccharides may be labelled radioactively, such as [¹⁴C] or [³H]. Such labelling may be inter alia useful in diagnostic applications and uses of the saccharides described herein. Such diagnostic applications and uses will be further described herein below.

In accordance with the inventive method, CP-X (or a functional derivative thereof) and the at least one donor carbohydrate may further be contacted with an acceptor carbohydrate during the contacting step of the in vitro method presented herein. Said acceptor carbohydrate may be oligomeric or polymeric CPS of Neisseria meningitidis serogroup X (X CPS), oligomeric or polymeric CPS of Neisseria meningitidis serogroup A (CPS A), and/or a carbohydrate structure containing terminal GlcNAc residues such as hyaluronic acid, heparin, heparin sulphate or protein-linked oligosaccharides. For example, a chimeric CPS obtainable by the in vitro method of the present invention comprising or composed of CPS or CPS-subunits of Neisseria meningitidis serogroups A/X or X/A can be synthesized. Said chimeric CPS comprising or composed of CPS or CPS-subunits of Neisseria meningitidis serogroups may contain a carbohydrate structure containing terminal GlcNAc residues such as hyaluronic acid, heparin, heparin sulphate or protein-linked oligosaccharides if used as an acceptor.

Within a chimeric CPS obtainable by the inventive in vitro method, one or more carbohydrates of the CPS-subunits may be derivatized and may contain, for example, additional functional groups such as amino groups, alkyl groups, hydroxyl groups, carboxylic acids, azides, amides, acetyl groups or halogen atoms; see also “Carbohydrate chemistry” Volumes 1-34 Cambridge (England), Royal Society of Chemistry, loc. cit. The chimeric CPS may contain one or more carbohydrates of each contained CPS-subunit. The sequence of the CPS-subunits of the chimeric CPS may be of any order.

As an example of the present invention, the in vitro method for producing capsular polysaccharides (CPS) of Neisseria meningitidis comprises the steps:
(a) contacting UDP-GlcNAc and hydrolysed A CPS with CP-X;
(b) incubation of UDP-GlcNAc and hydrolysed A CPS with CP-X; and
(c) isolating the artificial chimeric CPS composed of capsular polysaccharide subunits of Neisseria meningitidis serogroups A/X.

As mentioned above, also other combinations of activated or non-activated donor carbohydrates, acceptor carbohydrates and capsule polymerases (CP) as described herein can be applied. Such other combinations and other modifications do not defer from the gist of the present invention.

In another embodiment of the in vitro method presented herein, the capsular polymerase (CP) which is contacted with at least one donor carbohydrate is specific for synthesis of the CPS of Neisseria meningitidis serogroup A. Specifically, the CP contacted with at least one donor carbohydrate is CP-A or a functional derivative thereof. The nucleotide sequence encoding CP-A is shown in SEQ ID NO: 7. The amino acid sequence of CP-A is shown in SEQ ID NO: 8. A functional derivative of CP-A is an enzyme which is capable of synthesizing capsular polysaccharide of serogroup A (Swartley et al., J Bacteriol (1998), 180(6): 1533-1539). Preferably, in accordance with the present invention, the nucleotide sequence of a functional derivative of CP-A has a sequence identity to SEQ ID NO: 7 of at least 80%, more preferably at least 85%, more preferably at least 90%, and most preferably at least 95% and the amino acid sequence of a functional derivative of CP-A has a sequence identity to SEQ ID NO: 8 of at least 80%, more preferably at least 85%, more preferably at least 90%, more preferably at least 95%, and most preferably at least 99%. A functional derivative may also comprise a functional fragment maintaining the biological activity. Therefore, the term “functional derivative thereof” as used herein in context of nucleotide sequences or polypeptides refers to a functional fragment which has essentially the same (biological) activity as the nucleotide sequences or polypeptides defined herein (e.g. as shown in SEQ ID NO: 8) which may be encoded by the nucleic acid sequence of the present invention (e.g. SEQ ID NO: 7). The (biological) function can, inter alia, be assessed by the method described in Swartley et al., J Bacteriol (1998), 180(6): 1533-1539 as well as in the methods provided herein.

The term “functional derivative thereof” as used herein in context of nucleotide sequences or polypeptides refers to a functional fragment which has essentially the same (biological) activity as the nucleotide sequences or polypeptides defined herein (e.g. as shown in SEQ ID NO: 8) which may be encoded by the nucleic acid sequence of the present invention (e.g. SEQ ID NO: 7). Biological activity may be assessed by methods provided herein and known in the art; see, e.g., Swartley (1998), loc cit. Such functional derivatives comprise also functional fragments.

According to the present invention, identity levels of nucleotide or amino acid sequences refer to the entire length of nucleotide sequence of SEQ ID NO: 7 or polypeptide sequence of SEQ ID NO: 8, respectively and is assessed pair-wise, wherein each gap is to be counted as one mismatch. The term “identity” as used herein is used equivalently to the term “homology”. For example, the terms identity and homology are used herein in the context of a nucleic acid or a polypeptide/amino acid sequence which has an identity or homology of at least 80% to SEQ ID NO: 7 or 8, respectively, preferably over the entire length.

Accordingly, the present invention relates to the use of a polypeptide (being a CP-A or fragment thereof) in the present inventive method, wherein the polypeptide has at least 80% identity/homology to the polypeptide shown in SEQ ID NO: 8.

If, e.g., two nucleic acid sequences to be compared by, e.g., sequence comparisons differ in identity, then the term “identity” or “homology” refers to the shorter sequence and that part of the longer sequence that matches said shorter sequence. Therefore, when the sequences which are compared do not have the same length, the degree of identity preferably either refers to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that “matches” the shorter sequence. Also, these definitions for sequence comparisons (e.g., establishment of “identity” or “homology” values) are to be applied for all sequences described and disclosed herein. The terms “identity” and “homology” were further characterized hereinabove and the definitions and explanations apply, mutatis mutandis, for CP-A and functional fragments thereof.

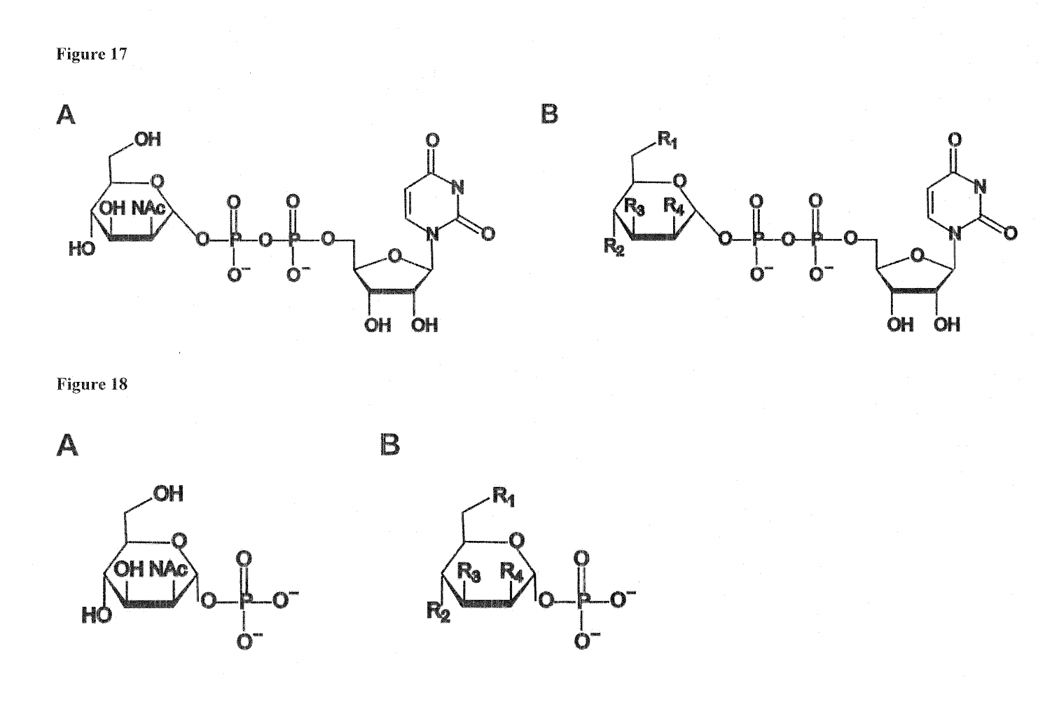

In one embodiment of the present in vitro method, the CP to be used is CP-A or a functional derivative thereof and at least one donor carbohydrate may be UDP-ManNAc or a derivative thereof. Examples for derivatives of UDP-ManNAc may be compounds that are alkylated or hydroxylated or that comprise additional functional groups such as carboxylic acids, azides, amides, acetyl groups or halogen atoms as also illustrated in FIG. 17B; see also “Carbohydrate chemistry” Volumes 1-34: monosaccharides, disaccharides, and specific oligosaccharides, Reviews of the literature published during 1967-2000, Cambridge (England), Royal Society of Chemistry.

In another embodiment of the in vitro method described herein, the capsule polymerase (CP) is CP-A or a functional derivative thereof and at least one donor carbohydrate is ManNAc-1-phosphate or a derivative thereof. Examples for derivatives of ManNAc-1-phosphate and sialic acid are illustrated in FIG. 18. Said donor carbohydrate ManNAc-1-phosphate may be contacted with at least one nucleotide and/or phosphoenolpyruvate (PEP) and auxiliary enzymes when contacted with the CP. Said nucleotide can be UMP, UDP and UTP. Said donor carbohydrate ManNAc-1-phosphate may be activated during incubation with the CP. In accordance with the herein presented in vitro method, this activation may yield the activated sugar nucleotide UDP-ManNAc, or its derivatives.

CP-A or a functional derivative thereof and the at least one donor carbohydrate may further be contacted with an acceptor carbohydrate during the contacting step of the inventive in vitro method. In accordance with the inventive in vitro method presented herein, the acceptor carbohydrate may be oligomeric or polymeric CPS of Neisseria meningitidis serogroup X (X CPS), oligomeric or polymeric CPS of Neisseria meningitidis serogroup A (CPS A) and/or a carbohydrate structure containing terminal GlcNAc or ManNAc residues such as hyaluronic acid, heparin, heparin sulphate or protein-linked oligosaccharides. For example, a chimeric CPS comprising or composed of CPS or CPS-subunits of Neisseria meningitidis serogroups A/X or X/A can be synthesized by the in vitro method presented herein. The chimeric CPS obtainable by the presented in vitro method comprising or composed of CPS or CPS-subunits of Neisseria meningitidis serogroups may contain a carbohydrate structure containing terminal GlcNAc residues such as hyaluronic acid, heparin, heparin sulphate or protein-linked oligosaccharides if used as an acceptor.

Within a chimeric CPS obtainable by the in vitro method of the present invention, one or more carbohydrates of the CPS-subunits may be derivatized and may contain, for example, additional functional groups such as amino groups, alkyl groups, hydroxyl groups, carboxylic acids, azides, amides, acetyl groups or halogen atoms; see also “Carbohydrate chemistry” Volumes 1-34 Cambridge (England), Royal Society of Chemistry; loc. cit. These chimeric CPS may contain one or more carbohydrates of each contained CPS-subunit. The sequence of the CPS-subunits of the chimeric CPS obtainable by the in vitro method described herein may be of any order.

As an example of the present invention, the in vitro method for producing capsular polysaccharides (CPS) of Neisseria meningitidis comprises the steps:
(a) contacting UDP-ManNAc and hydrolysed X CPS with CP-A;
(b) incubation of UDP-ManNAc and hydrolysed X CPS with CP-A; and
(c) isolating the artificial chimeric CPS composed of capsular polysaccharide subunits of Neisseria meningitidis serogroups X/A.

Again, the skilled person readily understands that also other combinations of activated or non-activated donor carbohydrates, acceptor carbohydrates and capsule polymerases (CP) as described herein can be applied. Such other combinations and other modifications do not defer from the gist of the present invention.

The acceptor carbohydrate which is contacted with the donor carbohydrate and the CP may be purified according to the in vitro method described herein. If said acceptor carbohydrate is oligomeric or polymeric CPS of Neisseria meningitidis, it may be hydrolysed.

The capsule polymerase (CP) contacted with the at least one donor carbohydrate in the presented in vitro method may be purified. Said CP may be isolated from Neisseria meningitidis lysates or recombinantly produced.

The present invention also relates to artificial chimeric CPS obtainable by the in vitro methods described herein. Such CPS may be synthetic or artificial chimeric CPS of Neisseria meningitidis serogroup W-135, Y, A, or X or artificial chimeric CPS comprising or composed of CPS of CPS-subunits of Neisseria meningitidis serogroups Y/W-135, W-135/Y, B/Y, C/Y, B/W-135, C/W-135, B/Y/W-135, C/Y/W-135, B/W-135/Y, C/W-135/Y, X/A or A/X.

The artificial chimeric CPS obtainable by the inventive in vitro method may be used as vaccines. In a preferred embodiment of the present invention, they are used in vaccination of a human subject. Also disclosed is the use of the chimeric CPS obtainable by the inventive in vitro method for the preparation of a vaccine. In a specific embodiment of the present invention, the chimeric CPS obtainable by the in vitro methods described herein are used as vaccines against meningococcal meningitidis caused by Neisseria meningitidis serogroup A, B, C, W-135, X or Y. The chimeric CPS obtainable by the in vitro methods may also be used for diagnosing meningococcal meningitidis caused by Neisseria meningitidis serogroup A, B, C, W-135, X or Y or diseases related thereto. The chimeric CPS obtainable by the in vitro methods can also be used in analytical procedures. For example, such a chimeric CPS may be used as defined standard carbohydrate to allow comparison with a sample carbohydrate to be analyzed.

The present invention further relates to antibodies binding to the artificial chimeric CPS obtainable by the in vitro methods described herein. Preferably, these antibodies specifically bind to the artificial chimeric CPS. The term “antibody” herein is used in the broadest sense and specifically encompasses intact monoclonal antibodies, polyclonal antibodies, multispecific antibodies (e.g., bispecific antibodies) formed from at least two intact antibodies, and antibody fragments, so long as they exhibit the desired biological activity. Also human and humanized as well as CDR-grafted antibodies are comprised.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monocional antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be constructed as requiring production of the antibody by any particular method. For example, the monoclonal antibodies to be used in accordance with the present invention may be made by the hybridoma method first described by Kohler, G. et al., Nature 256 (1975) 495, or may be made by recombinant DNA methods (see, e.g., U.S. Pat. No. 4,816,567). “Antibody fragments” comprise a portion of an intact antibody. In context of this invention, antibodies specifically recognize CPS or artificial chimeric CPS obtainable by the in vitro method described herein. Antibodies or fragments thereof as described herein may also be used in pharmaceutical and medical settings such as vaccination/immunization, particularly passive vaccination/immunization.

The antibodies of the present invention may also be used for treating and/or diagnosing meningococcal meningitidis caused by Neisseria meningitidis serogroup A, B, C, W-135, X or Y.

The present invention further relates to a pyrophosphorylase, particularly to the UDP-sugar phosphorylase (USP-LM) of Leishmania major (Damerow et al., J Biol Chem (2010), 285(2): 878-887). The nucleotide sequence of USP-LM is shown in SEQ ID NO: 9. The polypeptide sequence of USP-LM is shown in SEQ ID NO: 10. Said USP-LM is able to activate a hexose-1-phosphate and/or a pentose-1-phosphate into a nucleotide sugar. For example, the USP-LM activates galactose-1-phosphate (Gal-1-P) into UDP-galactose (UDP-Gal) and glucose-1-phosphate (Glc-1-P) into UDP-glucose (UDP-Glc). The activation may be reversible. USP-LM is further able to act on and activate a variety of hexose-1-phosphates as well as pentose-1-phosphates and hence presents a broad in vitro specificity. Examples for pentose-1-phosphates are xylose-1-phosphate, arabinose-1-phosphate, glucuronic acid-1-phosphate and there is also very weak activity on GlcNAc-1P.

Nucleic acid molecules encoding a pyrophosphorylase or a fragment thereof are also described herein. Such nucleic acid molecules may be DNA molecules, RNA molecules, oligonucleotide thiophosphates, substituted ribo-oligonucleotides or PNA molecules.

Furthermore, the term “nucleic acid molecule” may refer to DNA or RNA or hybrids thereof or any modification thereof that is known in the state of the art (see, e.g., U.S. Pat. No. 5,525,711, U.S. Pat. No. 4,711,955, U.S. Pat. No. 5,792,608 or EP 302175 for examples of modifications). The polynucleotide sequence may be single- or double-stranded, linear or circular, natural or synthetic, and without any size limitation. For instance, the polynucleotide sequence may be genomic DNA, cDNA, mRNA, antisense RNA, ribozymal or a DNA encoding such RNAs or chimeroplasts (Gamper, Nucleic Acids Research, 2000, 28, 4332-4339). Said polynucleotide sequence may be in the form of a plasmid or of viral DNA or RNA. In particular, the present invention relates to a nucleic acid molecule having the nucleotide sequence of SEQ ID NO: 9. The present invention also encompasses nucleic acid molecules comprising the nucleic acid molecule of SEQ ID NO: 9 wherein one, two, three or more nucleotides are added, deleted or substituted. Such a nucleic acid molecule may encode a polypeptide having pyrophosphorylase activity. The term “activity” as used herein refers in particular to the capability of polypeptides or fragments thereof to activate sugar-1-phosphates into nucleotide sugars. In a specific embodiment of the present invention, the nucleic acid molecule described herein encodes a polypeptide which is able to activate a hexose-1-phosphate and/or a pentose-1-phosphate into a nucleotide sugar, particularly galactose-1-phosphate (Gal-1-P) into UDP-galactose (UDP-Gal) and glucose-1-phosphate (Glc-1-P) into UDP-glucose (UDP-Glc). The activation may be reversible. The person skilled in the art can easily determine the activity of a polypeptide to activate sugar-1-phosphates into nucleotide sugars. The synthesis of UDP-Glc, UDP-Gal or other UDP-sugars from their respective sugar-1-phosphates and UTP (forward reaction) generates pyrophosphate as by-product which can be monitored using for example the Enz-Chek Pyrophosphate Kit (Invitrogen). Alternatively, the formation of UTP may be followed to analyze the synthesis of sugar-1-phosphates from nucleotide sugars and pyrophosphate (reverse reaction). In this assay, E. coli CTP-synthase (31) may be used to generate free inorganic phosphate from UTP which may again be detected using the Enz-Chek Pyrophosphate Kit (Invitrogen) or Enz-Chek Phosphate Kit (Invitrogen). Details are given illustratively in example 11. Preferably, the nucleic acid molecule described in the present invention is of at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98% and most preferably at least 99% identical to SEQ ID NO: 9. This nucleic acid molecule preferably encodes a polypeptide which is able to activate a hexose-1-phosphate and/or a pentose-1-phosphate into a nucleotide sugar, particularly galactose-1-phosphate (Gal-1-P) into UDP-galactose (UDP-Gal) and glucose-1-phosphate (Glc-1-P) into UDP-glucose (UDP-Glc). The activation may be reversible.

The present invention further relates to nucleic acid molecules which are complementary to the nucleic acid molecules described above. Also encompassed are nucleic acid molecules which are able to hybridize to nucleic acid molecules described herein. A nucleic acid molecule of the present invention may also be a fragment of the nucleic acid molecules described herein. Particularly, such a fragment is a functional fragment. Examples for such functional fragments are nucleic acid molecules which can serve as primers.

The term “hybridization” or “hybridizes” as used herein in context of nucleic acid molecules/DNA sequences may relate to hybridizations under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

In accordance to the invention described herein, low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

Hybridizing nucleic acid molecules also comprise fragments of the above described molecules. Such fragments may represent nucleic acid molecules which code for a functional pyrophosphorylase as described above or a functional fragment thereof which can serve as primers. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions which depend upon binding between nucleic acids strands.

The term “hybridizing sequences” preferably refers to sequences which display a sequence identity of at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98% and most preferably at least 99% identity with a nucleic acid sequence as described above encoding a pyrophosphorylase. Moreover, the term “hybridizing sequences” preferably refers to sequences encoding a pyrophosphorylase as described above having a sequence identity of at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, and most preferably at least 99% identical to SEQ ID NO: 10.

The present invention further relates to vectors containing a nucleic acid molecule of the present invention encoding a pyrophosphorylase. The present invention relates also to a vector comprising the nucleic acid construct encoding the herein described pyrophosphorylase. The term “vector” as used herein particularly refers to plasmids, cosmids, viruses, bacteriophages and other vectors commonly used in genetic engineering. In a preferred embodiment, the vectors of the invention are suitable for the transformation of cells, like fungal cells, cells of microorganisms such as yeast or prokaryotic cells. In a particularly preferred embodiment such vectors are suitable for stable transformation of bacterial cells, for example to express the pyrophosphorylase of the present invention.

Accordingly, in one aspect of the invention, the vector as provided is an expression vector. Generally, expression vectors have been widely described in the literature. As a rule, they may not only contain a selection marker gene and a replication-origin ensuring replication in the host selected, but also a promoter, and in most cases a termination signal for transcription. Between the promoter and the termination signal there is preferably at least one restriction site or a polylinker which enables the insertion of a nucleic acid sequence/molecule desired to be expressed

It is to be understood that when the vector provided herein is generated by taking advantage of an expression vector known in the prior art that already comprises a promoter suitable to be employed in context of this invention, for example expression of a pyrophosphorylase as described herein above, the nucleic acid construct is inserted into that vector in a manner the resulting vector comprises only one promoter suitable to be employed in context of this invention. The skilled person knows how such insertion can be put into practice. For example, the promoter can be excised either from the nucleic acid construct or from the expression vector prior to ligation.

A non-limiting example of the vector of the present invention is the plasmid vector pET22b comprising the nucleic acid construct of the present invention. Further examples of vectors suitable to comprise the nucleic acid construct of the present invention to form the vector of the present invention are known in the art and are, for example other vectors for bacterial expression systems such as vectors of the pET series (Novagen) or pQE vectors (Qiagen).

In an additional embodiment, the present invention relates to a host cell comprising the nucleic acid construct and/or the vector of the present invention. Preferably, the host cell of the present invention may be a prokaryotic cell, for example, a bacterial cell. As a non limiting example, the host cell of the present invention may be Escherichia coli. The host cell provided herein is intended to be particularly useful for generating the pyrophosphorylase of the present invention.

Generally, the host cell of the present invention may be a prokaryotic or eukaryotic cell, comprising the nucleic acid construct or the vector of the invention or a cell derived from such a cell and containing the nucleic acid construct or the vector of the invention. In a preferred embodiment, the host cell comprises, i.e. is genetically modified with, the nucleic acid construct or the vector of the invention in such a way that it contains the nucleic acid construct of the present invention integrated into the genome. For example, such host cell of the invention, but also the host cell of the invention in general, may be a bacterial, yeast, or fungus cell.

In one particular aspect, the host cell of the present invention is capable to express or expresses a pyrophosphorylase as defined herein and as illustrative characterized in SEQ ID NO: 10. An overview of examples of different corresponding expression systems to be used for generating the host cell of the present invention, for example this particular one, is for instance contained in Methods in Enzymology 153 (1987), 385-516, in Bitter et al. (Methods in Enzymology 153 (1987), 516-544), in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), and in Griffiths et al., (Methods in Molecular Biology 75 (1997), 427-440).

The transformation or genetically engineering of the host cell with a nucleic acid construct or vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990.

Further described herein are polypeptides comprising the amino acid sequence of SEQ ID NO: 10 wherein one, two, three or more amino acid residues are added, deleted or substituted. The polypeptide may have the function of a pyrophosphorylase. Preferably, the polypeptide is able to activate a hexose-1-phosphate and/or a pentose-1-phosphate into a nucleotide sugar, particularly galactose-1-phosphate (Gal-1-P) into UDP-galactose (UDP-Gal) and glucose-1-phosphate (Glc-1-P) into UDP-glucose (UDP-Glc). The activation may be reversible. The amino acid sequence of the polypeptide may be at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, and most preferably at least 99% identical to SEQ ID NO: 10. Preferably, the polypeptide is able to activate a hexose-1-phosphate and/or a pentose-1-phosphate into a nucleotide sugar, particularly galactose-1-phosphate (Gal-1-P) into UDP-galactose (UDP-Gal) and glucose-1-phosphate (Glc-1-P) into UDP-glucose (UDP-Glc). The activation may be reversible. Also encompassed are functional fragments of the polypeptides described herein. Functional fragments of these polypeptides exhibit pyrophosphorylase functions. Preferably, these functional fragments are able to activate a hexose-1-phosphate and/or a pentose-1-phosphate into a nucleotide sugar, particularly galactose-1-phosphate (Gal-1-P) into UDP-galactose (UDP-Gal) and glucose-1-phosphate (Glc-1-P) into UDP-glucose (UDP-Glc). The activation may be reversible.

The nucleic acid molecules or fragments thereof as well as the vectors, host cells and polypeptides or fragments thereof described herein may further be used for activating a hexose-1-P and/or a pentose-1-P. In accordance with the present invention, such a use may be in vitro. Examples for hexose-1-P are Glc-1-P or Gal-1-P. Examples for pentose-1-P are xylose-1-P or arabinose-1-P.

Identity levels of nucleotide or amino acid sequences refer to the entire length of nucleotide sequence of SEQ ID NO: 9 or polypeptide sequence of SEQ ID NO: 10, respectively and is assessed pair-wise, wherein each gap is to be counted as one mismatch. The term “identity” as used herein is used equivalently to the term “homology”. For example, this ter: is used herein in the context of a nucleic acid sequence which has a homology, that is to say a sequence identity, of at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, and most preferably of at least 99% to another, preferably entire, nucleic acid sequence.

As regards amino acid/polypeptide sequences or fragments thereof, this term is used herein in the context of amino acid/polypeptide sequences or fragments thereof which have a homology, that is to say a sequence identity, of at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, and most preferably at least 99% identical to another, preferably entire, amino acid/polypeptide sequence.

Accordingly, the present invention relates to a pyrophosphorylase or fragment thereof of at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, and most preferably at least 99% identity/homology to the polypeptide shown in SEQ ID NO: 10.

Also in context of this embodiment relating to the herein disclosed pyrophosphorylase (or a functional fragment thereof), if, e.g., two nucleic acid sequences to be compared by, e.g., sequence comparisons differ in identity, then the term “identity” or “homology” refers to the shorter sequence and that part of the longer sequence that matches said shorter sequence. Therefore, when the sequences which are compared do not have the same length, the degree of identity preferably either refers to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that “matches” the shorter sequence. Also, these definitions for sequence comparisons (e.g., establishment of “identity” or “homology” values) are to be applied for all sequences described and disclosed herein.

Also in context of the novel pyrophosphorylase as presented herein, the identity means that there is a functional and/or structural equivalence between the corresponding nucleotide sequence or polypeptides, respectively (e.g., polypeptides encoded thereby). Nucleic acid/amino acid sequences having the given identity levels to the herein-described particular nucleic acid/amino acid sequences may represent derivatives/variants of these sequences which, preferably, have the same biological function. They may be either naturally occurring variations, for instance sequences from other varieties, species, etc., or mutations, and said mutations may have formed naturally or may have been produced by deliberate mutagenesis. Furthermore, the variations may be synthetically produced sequences. The allelic variants of the herein disclosed pyrophosphorylase may be naturally occurring variants or synthetically produced variants or variants produced by recombinant DNA techniques. Deviations from the above-described nucleic acid sequences may have been produced, e.g., by deletion, substitution, addition, insertion and/or recombination. The term “addition” refers to adding at least one nucleic acid residue/amino acid to the end of the given sequence, whereas “insertion” refers to inserting at least one nucleic acid residue/amino acid within a given sequence. The term “deletion” refers to deleting or removal at least one nucleic acid residue /amino acid residue in a given sequence. The term “substitution” refers to the replacement of at least one nucleic acid residue/amino acid residue in a given sequence.

The variant polypeptides of the herein disclosed pyrophosphorylase and, in particular, the polypeptides encoded by the different variants of the nucleic acid sequences of the invention preferably exhibit certain characteristics they have in common. These include, for instance, biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

The term “synthetic” as used herein describes a CPS structure which is synthesized in vitro and wherein the CPS has identical structure to the structure found in native CPS of Neisseria meningitidis.

The term “artificial” as used herein describes a CPS structure which is synthesized in vitro and which is not identical to structures found in native CPS of Neisseria meningitidis. For example, an artificial CPS is a chimeric CPS comprising or composed of two or more CPS-subunits of Neisseria meningitidis serogroups A, B, C, W-135, X and/or Y or a CPS which comprises one or more derivatized building blocks of different CPS of Neisseria meningitidis serogroups A, B, C, W-135, X and/or Y. Examples for such derivatized building blocks are shown in FIGS. 1 to 5. A chimeric CPS may comprise or be composed of CPS or CPS-subunits of Neisseria meningitidis serogroups Y/W-135, W-135/Y, B/Y, C/Y, B/W-135, C/W-135, B/Y/W-135, C/Y/W-135, B/W-135/Y C/135/Y, X/A or A/X. Within a chimeric CPS, one or more building blocks of the CPS-subunits may be derivatized as exemplarily shown in FIGS. 1 to 5. A chimeric CPS may contain one or more carbohydrates of each contained CPS-subunit. The sequence of the CPS-subunits of a chimeric CPS may be of any order. Examples for chimeric CPS are illustrated in FIG. 6.

The term “carbohydrate” as used herein comprises building blocks such as saccharides and sugars in any form as well as aldehydes and ketones with several hydroxyl groups added. A carbohydrate may contain one or more of said building blocks linked via covalent bonds such as glycosidic linkages. A carbohydrate may be of any length, i.e. it may be monomeric, dimeric, trimeric or multimeric. A carbohydrate may also contain one or more building blocks as side chains linked to the main chain via covalent bonds. A carbohydrate may also contain one or more activated saccharides such as nucleotide sugars. Examples of nucleotide sugars are UDP-Glc, UDP-Gal, UDP-GlcNAc, UDP-GlcUA, UDP-Xyl, GDP-Man, GDP-Fuc, CMP-Neu5Ac and CMP-NeuNAc.

The term “CPS-subunit” as used herein describes one or more carbohydrates specific for a respective CPS of a Neisseria meningitidis serogroup. Within a CPS-subunit, one or more carbohydrates may be derivatized. If two or more carbohydrates are present within one particular CPS-subunit, they are linked by linkages which are specific for the CPS of the respective Neisseria meningitidis serogroup.

It is evident form the above, that the present invention provides for means and methods for the generation of synthetic capsular polysaccharides and, in particular, artificial chimeric capsular polysaccharides. Accordingly, the present invention also relates to chimeric capsular polysaccharides, in particular of Neisseria meningitidis that are obtained or are obtainable by the method provided herein. Such chimeric capsular polysaccharides are, inter alia, chimeric capsular polysaccharides comprising capsular polysaccharides or capsular polysaccharide subunits of Neisseria meningitidis serogroups Y/W-135, W-135/Y, B/Y, C/Y, B/W-135, C/W-135, B/Y/W-135, C/Y/W-135, B/W-135/Y, C/W-135/Y, X/A or A/X.

Such capsular polysaccharides as provided herein are not only useful as scientific tools but are also very valuable in medical settings, for example as pharmaceutical compositions. Such pharmaceutical compositions may comprise vaccines. Accordingly, the present invention also relates to pharmaceutical compositions comprising the chimeric capsular polysaccharides described herein. Said capsular polysaccharides may be isolated but it is also envisaged that these chimeric capsular polysaccharides are to be used in context with other structures, e.g., polypeptides and the like. Such polypeptides may, inter alia, function as carriers or backbones for the herein described inventive chimeric capsular polysaccharides. Numerous methods have been developed to link oligosaccharides covalently to proteins (Lit: (a) Vince Pozsgay, Oligosaccharide-protein conjugates as vaccine candidates against bacteria, Advances in Carbohydrate Chemistry and Biochemistry, Academic Press, 2000, Volume 56, Pages 153-199, (b) Jennings, H. J, R. K. Sood (1994) Synthetic glycoconjugates as human vaccines; in Lee, Y. C. R. T. Lee (eds): Neoglycoconjugates. Preparation and Applications. San Diego, Academic Press, pp 325-371, (c) Pozsgay, V.; Kubler-Kielb, J., Conjugation Methods toward Synthetic Vaccines, Carbohydrate-Based Vaccines, American Chemical Society, Jul. 2, 2008, 36-70); (D) Carl E. Frasch, Preparation of bacterial polysaccharide-protein conjugates: Analytical and manufacturing challenges, Vaccine, In Press, Corrected Proof, Available online 24 Jun. 2009, ISSN 0264-410X, DOI: 10.1016/j.vaccine.2009.06.013.) One example is the covalent coupling of the synthetic or artificial CPS molecules described herein to protein amino-groups by means of reductive amination.

Therefore, the present invention also comprises compounds that comprise the chimeric capsular polysaccharide as described herein. Such compounds are of particular scientific as well as medical use. One of such uses is the use as a vaccine, i.e. the compounds provided herein can be employed for the vaccination of a subject. Such a subject may be a mammal and, in a particular embodiment, a human being. The vaccines provided herein are particularly useful in the vaccination against Neisseria. In accordance with the above, the present invention also provides for the use of a compound comprising the chimeric capsular polysaccharide disclosed herein for the preparation of a vaccine to be administered to a subject, preferably to a mammal and most preferably to a human being. Such a medical use in particular relates to the medical use or intervention of disorders, like in the vaccination against meningitis, in particular against meningococcal meningitidis caused by Neisseria meningitidis serogroup A, B, C, W-135, X or Y.

However, as mentioned above and as illustrated in the appended examples, the present invention also relates to a novel pyrophosphoryase (Damerow et al. Biol Chem (2010), 285(2): 878-887). Accordingly, the present invention also provides for the use of the herein defined pyrophosphorylase in scientific research, in industrial settings as well as in medical settings. The invention, therefore, also relates to the use of a nucleic acid molecule encoding for the herein defined pyrophosphorylase (or a functional fragment thereof), a vector comprising such a nucleic acid molecule, a host cell comprising such a nucleic acid molecules or such a vector, or the herein defined pyrophosphorylase (or a functional fragment thereof) itself for activating a hexose-1-phosphate and/or a pentose-1-phosphate into a nucleotide sugar. Said hexose-1-phosphate may, inter alia, be selected from the group consisting of: Glc-1P and Gal-1-P and the pentose-1-phosphate may, inter alia, be selected from the group consisting of: xylose-1-P and arabinose-1-P.

Such a use of the herein disclosed pyrophosphorylase can be an in vitro use. The use of the pyrophosphorylase as described herein is in particular envisaged in (bio)chemical processes and methods as disclosed herein, e.g., in the production of synthetic polysaccharides, like chimeric capsular polysaccharides. The herein described pyrophosphorylase can also be used in the production of activated nucleotide sugars such as UDP-Gal, UDP-Glc, UDP-Xyl, UDP-GalA or UDP-Ara.

The compositions provided herein may comprise the synthetic and/or chimeric polysaccharides (CPS) as described herein. Such compositions are useful, inter alia, for medical and diagnostic purposes, in particular, for pharmaceutical and vaccination purposes, i.e. for the treatment or the diagnostic detection of Neisseria-induced diseases or the vaccination against these pathogens. Therefore, the present invention also relates to a composition as defined above which is a pharmaceutical composition further comprising, optionally, a pharmaceutically acceptable carrier.

The pharmaceutical composition of the present invention may comprise the CPS of the present invention. The pharmacological composition may further comprise the antibodies specifically directed against these CPS of the present invention, e.g., antibodies (or their fragments or derivatives) of the invention which are directed against these synthetic CPS disclosed herein or which were generated against these CPS. Such CPS as well as the antibodies directed against the same may be used, inter alia, in vaccination protocols, either alone or in combination. Therefore, the pharmaceutical composition of the present invention comprising the CPS of this invention or antibodies directed against the same, may be used for pharmaceutical purposes such as effective therapy of infected humans and animals and/or for vaccination purposes. Accordingly, the present invention relates to pharmaceutical compositions comprising the CPS as described herein and/or antibodies or antibody fragments against the CPS as described herein and, optionally, a pharmaceutically acceptable carrier. In context with the present invention, the pharmaceutical compositions described herein may be used, inter alia, for the treatment, prevention and/or diagnostic of Neisseria-induced diseases and/or infections. Preferably, the pharmaceutical composition is used as a vaccine as will be further described herein below.

The pharmaceutical composition of the present invention may further comprise a pharmaceutically acceptable carrier, excipient and/or diluent. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The pharmaceutical composition of the present invention, particularly when used for vaccination purposes, may be employed at about 0.01 μg to 1 g CPS per dose, or about 0.5 μg to 500 μg CPS per dose, or about 1 μg to 300 μg CPS per dose. However, doses below or above this exemplary range are envisioned, especially considering the aforementioned factors. Administration of the suitable compositions may be effected by different ways, e.g., by intravenous, intraperitoneal, subcutaneous, intramuscular, topical or intradermal administration. However, in particular in the pharmaceutical intervention of the present invention, Neisseria infections can demand an administration to the side of infection, like the brain. Progress can be monitored by periodic assessment. The compositions of the invention may be administered locally or systemically. Administration will generally be parenterally, e.g., intravenously. The compositions of the invention may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins and/or interferons depending on the intended use of the pharmaceutical composition.

In a preferred embodiment of the present invention, the pharmaceutical composition as defined herein is a vaccine.

Vaccines may be prepared, inter alia, from one or more CPS as described herein, or from one or more antibodies, fragments of said antibodies or derivatives of the antibodies of the invention, i.e. antibodies against the CPS as disclosed herein. Accordingly, in context with the present invention, vaccines may comprise one or more CPS as described herein and/or one or more antibodies, fragments of said antibodies or derivatives of the antibodies of the invention, i.e. antibodies against the CPS as disclosed herein.

The CPS or the antibodies, fragments or derivatives of said antibodies of the invention used in a pharmaceutical composition as a vaccine may be formulated, e.g., as neutral or salt forms. Pharmaceutically acceptable salts, such as acid addition salts, and others, are known in the art. Vaccines can be, inter alia, used for the treatment and/or the prevention of an infection with pathogens, e.g. Neisseria, and are administered in dosages compatible with the method of formulation, and in such amounts that will be pharmacologically effective for prophylactic or therapeutic treatments.

A vaccination protocol can comprise active or passive immunization, whereby active immunization entails the administration of an antigen or antigens (like the chimeric polysaccharides of the present invention or antibodies, fragments of said antibodies or derivatives of the antibodies specifically directed against these CPS) to the host/patient in an attempt to elicit a protective immune response. Passive immunization entails the transfer of preformed immunoglobulins or derivatives or fragments thereof (e.g., the antibodies, the derivatives or fragments thereof of the present invention, i.e. specific antibodies directed against the chimeric CPS of this invention and as obtained by the means and methods provided herein) to a host/patient. Principles and practice of vaccination and vaccines are known to the skilled artisan, see, for example, in Paul, “Fundamental Immunology” Raven Press, New York (1989) or Morein, “Concepts in Vaccine Development”, ed: S. H. E. Kaufmann, Walter de Gruyter, Berlin, N.Y. (1996), 243-264; Dimitriu S, editor. “Polysaccharides in medicinal application”; New York: Marcel Dekker, pp 575-602. Typically, vaccines are prepared as injectables, either as liquid solutions or suspensions; solid forms suitable for solution in or suspension in liquid prior to injection also may be prepared. The preparation may be emulsified or the protein may be encapsulated in liposomes. The active immunogenic ingredients often are mixed with pharmacologically acceptable excipients which are compatible with the active ingredient. Suitable excipients include but are not limited to water, saline, dextrose, glycerol, ethanol and the like; combinations of these excipients in various amounts also may be used. The vaccine also may contain small amounts of auxiliary substances such as wetting or emulsifying reagents, pH buffering agents, and/or adjuvants which enhance the effectiveness of the vaccine. For example, such adjuvants can include aluminum compositions, like aluminumhydroxide, aluminumphosphate or aluminumphosphohydroxide (as used in “Gen H-B-Vax®” or “DPT-Impfstoff Behring”), N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1∝2′-dipalmitoyl-sn-glycero-3-hydroxyphaosphoryloxy)-ethylamine (CGP 19835A, also referred to as MTP-PE), MF59 and RIBI (MPL+TDM+CWS) in a 2% squalene/Tween-80® emulsion.

The vaccines usually are administered by intravenous or intramuscular injection. Additional formulations which are suitable for other modes of administration include suppositories and, in some cases, oral formulations. For suppositories, traditional binders and carriers may include but are not limited to polyalkylene glycols or triglycerides. Oral formulation include such normally employed excipients as, for example, pharmaceutical grades of mannitol, lactose, starch, magnesium stearate, sodium saccharine, cellulose, magnesium carbonate and the like. These compositions may take the form of solutions, suspensions, tables, pills, capsules, sustained release formulations or powders and contain about 10% to about 95% of active ingredient, preferably about 25% to about 70%.

Vaccines are administered in a way compatible with the dosage formulation, and in such amounts as will be prophylactically and/or therapeutically effective. The quantity to be administered generally is in the range of about 0.01 μg to 1 g antigen per dose, or about 0.5 μg to 500 μg antigen per dose, or about 1 μg to 300 μg antigen per dose (in the present case CPS being the antigen), and depends upon the subject to be dosed, the capacity of the subject's immune system to synthesize antibodies, and the degree of protection sought. Precise amounts of active ingredient required to be administered also may depend upon the judgment of the practitioner and may be unique to each subject. The vaccine may be given in a single or multiple dose schedule. A multiple dose is one in which a primary course of vaccination may be with one to ten separate doses, followed by other doses given at subsequent time intervals required to maintain and/or to reinforce the immune response, for example, at one to four months for a second dose, and if required by the individual, a subsequent dose(s) after several months. The dosage regimen also will be determined, at least in part, by the need of the individual, and be dependent upon the practitioner's judgement. It is contemplated that the vaccine containing the immunogenic compounds of the invention may be administered in conjunction with other immunoregulatory agents, for example, with immunoglobulins, with cytokines or with molecules which optimize antigen processing, like listeriolysin.

For diagnosis and quantification of pathogens like Neisseria, pathogenic fragments, their derivatives, their (poly)peptides (proteins), their polynucleotides, etc. in clinical and/or scientific specimens, a variety of immunological methods, as well as molecular biological methods, like nucleic acid hybridization assays, PCR assays or DNA Enzyme Immuno Assays (DEIA; Mantero et al., Clinical Chemistry 37 (1991), 422-429) have been developed and are well known in the art. In this context, it should be noted that the nucleic acid molecules of the invention may also comprise PNAs, modified DNA analogs containing amide backbone linkages. Such PNAs are useful, inter alia, as probes for DNA/RNA hybridization. The proteins of the invention may be, inter alia, useful for the detection of anti-pathogenic (like, e.g., anti-bacterial or anti-viral) antibodies in biological test samples of infected individuals. It is also contemplated that antibodies and compositions comprising such antibodies of the invention may be useful in discriminating acute from non-acute infections. The CPS as provided herein can also be used in diagnostic settings, for example as “standards”, in, e.g., chromatographic approaches. Therefore, the present CPS can be used in comparative analysis and can be used either alone or in combination to diagnostic methods known in the art.

The diagnostic composition optionally comprises suitable means for detection. The CPS as disclosed and described herein as well as specific antibodies or fragments or derivatives thereof directed or raised specifically against these chimeric polysaccharides are, for example, suitable for use in immunoassays in which they can be utilized in liquid phase or bound to a solid phase carrier. Solid phase carriers are known to those in the art and may comprise polystyrene beads, latex beads, magnetic beads, colloid metal particles, glass and/or silicon chips and surfaces, nitrocellulose strips, membranes, sheets, animal red blood cells, or red blood cell ghosts, duracytes and the walls of wells of a reaction tray, plastic tubes or other test tubes. Suitable methods of immobilizing nucleic acids, (poly)peptides, proteins, antibodies, microorganisms etc. on solid phases include but are not limited to ionic, hydrophobic, covalent interactions and the like. Examples of immunoassays which can utilize said proteins, antigenic fragments, fusion proteins, antibodies or fragments or derivatives of said antibodies of the invention are competitive and non-competitive immunoassays in either a direct or indirect format. Commonly used detection assays can comprise radioisotopic or non-radioisotopic methods. Examples of such immunoassays are the radioimmunoassay (RIA), the sandwich (immunometric assay) and the Western blot assay. Furthermore, these detection methods comprise, inter alia, IRMA (Immune Radioimmunometric Assay), EIA (Enzyme Immuno Assay), ELISA (Enzyme Linked Immuno Assay), FIA (Fluorescent Immuno Assay), and CLIA (Chemioluminescent Immune Assay). Other detection methods that are used in the art are those that do not utilize tracer molecules. One prototype of these methods is the agglutination assay, based on the property of a given molecule to bridge at least two particles.

The CPS of the invention can be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, an magnetite. The nature of the carrier can be either soluble or insoluble for the purposes of the invention.

A variety of techniques are available for labeling biomolecules, are well known to the person skilled in the art and are considered to be within the scope of the present invention and comprise, inter alia, covalent coupling of enzymes or biotinyl groups, iodinations, phosphorylations, biotinylations, random priming, nick-translations, tailing (using terminal transferases) or labeling of carbohydrates. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc., or in Fotini N. Lamari, Reinhard Kuhn, Nikos K. Karamanos, “Derivatization of carbohydrates for chromatographic, electrophoretic and mass spectrometric structure analysis”, Journal of Chromatography B, Volume 793, Issue 1, Derivatization of Large Biomolecules, (2003), Pages 15-36.

Detection methods comprise, but are not limited to, autoradiography, fluorescence microscopy, direct and indirect enzymatic reactions, etc.

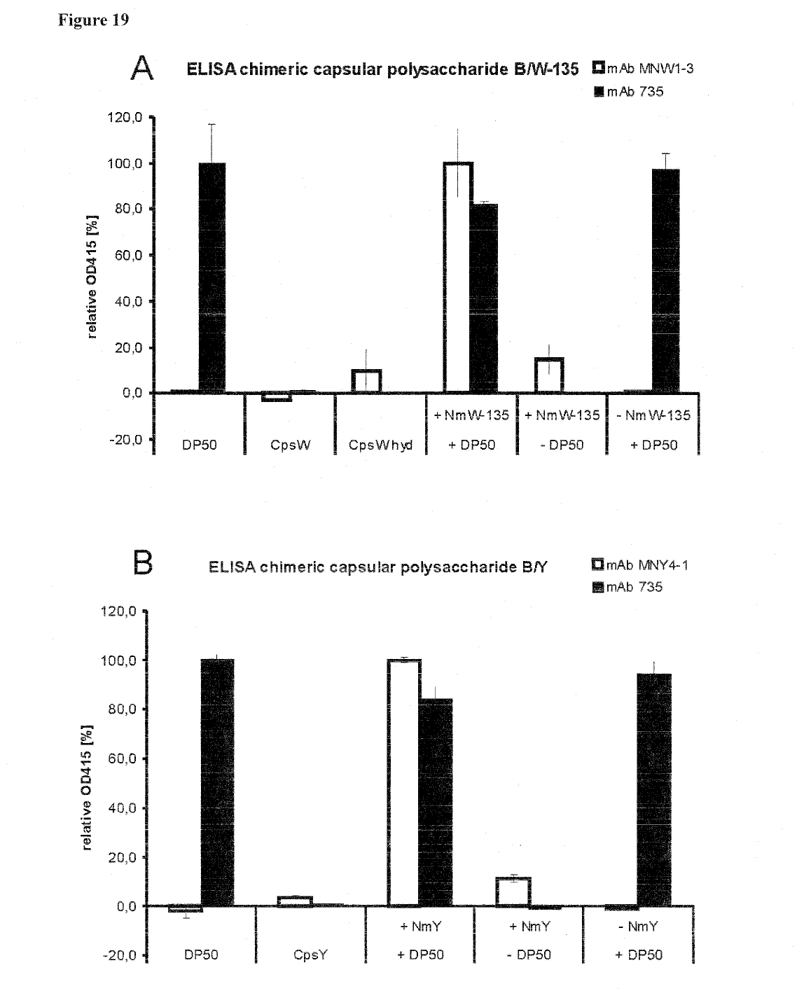

The chimeric CPS described herein may be detected by methods known the art as well as described and exemplified herein. For example, an ELISA (Enzyme-linked immunosorbent assay) based method described herein may be used for the detection and quantification of the chimeric CPS described herein. In this context, the chimeric CPS described herein may be immobilized by an antibody or other binding molecule, such as a lectine or similar, contacting one part or building block of the chimeric CPS. Detection of a second part or building block of the chimeric CPS described herein can be achieved by, e.g., contacting with an antibody or other binding molecule as described herein which is labeled for further detection or a secondary antibody or other binding molecule as described which is labeled for further detection. Labeling molecules suitable for this purpose are described and exemplified herein above and below. Examples for the detection of chimeric CPS described herein and obtainable by the method provided herein are illustrated in FIG. 19 or described in the Examples below, particularly Examples 14 and 15.

The invention relates further to a method for the production of a vaccine against a strain genus Neisseria comprising the steps of:
(a) Synthetic or in vitro production of (a) polysaccharide(s) as defined above; and
(b) combining said (a) polysaccharide(s) with a pharmaceutically acceptable carrier.

In a preferred embodiment of this method for the production of a vaccine, said “polysaccharide(s)” is/are (a) chimeric CPS as disclosed herein.

Furthermore, the invention relates to a method for the production of a vaccine against a strain or strains of the genus Neisseria, in particular N. meningitidis by combining (a) polysaccharide(s) (preferably (a) chimeric polysaccharide(s)) of the invention with a biologically acceptable carrier.

The Figures show:

FIG. 1: Schematic representation of UDP-Gal, CMP-Neu5Ac and possible derivatives thereof. A) UDP-galactose; B) potential target-sites for derivatisations of UDP-galactose are represented by R₁, R₂, R₃ and R₄. Examples for R₁₋₄ are: R═H, R═OH, R═N₃, R═F, R═(CH₂)ₓN₃, R═COOH R═(CH₂)ₓCOOH, R═NH(O)CH₃, R═NH(CO)(CH₂)ₓCH₃, R═O(CO)CH₃, R═O(CO)(CH₂)ₓCH₃; C) CMP-sialic acid; D) potential target sites for derivatisations of CMP-sialic acid are represented by R₁, R₂, R₃, R₄ and R₅. Examples for R₁₋₅ are: R═H, R═OH, R═N₃, R═F, R═(CH₂)ₓN₃, R═COOH, R═(CH₂)ₓCOOH, R═NH(CO)CH₃, R═NH(CO)(CH₂)ₓCH₃, R═O(CO)CH₃, R═O(CO)(CH₂)ₓCH₃.

FIG. 2: Schematic representation of UDP-Glc and possible derivatives thereof. A) UDP-glucose; B) potential target-sites for derivatisations of UDP-glucose are represented by R₁, R₂, R₃ and R₄. Examples for R₁₋₄ are: R═H, R═OH, R═N₃, R═F, R═(CH₂)ₓN₃, R═COOH, R═(CH₂)ₓCOOH, R═NH(CO)CH₃, R═NH(CO)(O)(CH₂)CH₃, R═O(CO)CH₃, R═O(CO)(CH₂)ₓCH₃.

FIG. 3: Schematic representation of UDP-GlcNAc and possible derivatives thereof. A) UDP-GlcNAc, B) potential target-sites for derivatisations of UDP-GlcNAc are represented by R₁, R₂, R₃ and R₄. Examples for R₁₋₄ are: R═H, R═OH, R═N₃, R═F, R═(CH₂)ₓN₃, R═COOH, R═(CH₂)ₓCOOH R═NH(CO)CH₃, R═NH(CO)(CH₂)ₓCH₃, R═O(CO)CH₃, R═O(CO)(CH₂)ₓCH₃.

FIG. 4: Schematic representation of Gal-1-P, sialic acid and possible derivatives thereof. A) galactose-1-phosphate; B) potential target-sites for derivatisations of Gal-1-P are represented by R₁, R₂, R₃ and R₄. Examples for R₁₋₄ are: R═H, R═OH, R═N₃, R═F, R═(CH₂)ₓN₃, R═COOH, R═(CH₂)ₓCOOH R═NH(CO)CH₃, R═NH(CO)(CH₂)ₓCH₃, R═O(CO)CH₃, R═O(CO)(CH₂)ₓCH₃; C) N-Acetylneuraminic acid; D) potential target sites for derivatisations of N-Acetylneuraminic acid represented by R₁, R₂, R₃ and R₄. Examples for R₁₋₄ are: R═H, R═OH, R═N₃, R═F, R═(CH₂)ₓN₃, R═COOH, R═(CH₂)ₓCOOH, R═NH(CO)(CH₃, R═N(CO)(CH₂)ₓCH₃, R═O(O)CH₃, ═O(CO)(CH₂)ₓCH₃.

FIG. 5: Schematic representation of acceptor derivatives. A) Terminal sugar at the reducing end of oligomeric/polymeric serogroup W-135 or Y capsular polysaccharide that carries a functional group attached to the anomeric carbon C2. R₁═OH, R₁=[→2)-α-Neu5Ac-(8→]ₓ, R₁=[→2)-α-Neu5Ac-(9→]ₓ, R₁=[→1]-α-D-Glc-(6→2)-α-Neu5Ac(4→]ₓ, R₁=→1)-α-D-Gal-(6→+2)-α-Neu5Ac(4→]ₓ; B) Terminal sugar at the reducing end of oligomeric/polymeric serogroup B capsular polysaccharide that carries a functional group attached to the anomeric carbon C2. R₂═OH, R₂=[→2)-α-Neu5Ac-(8→)ₓ]; C) Terminal sugar at the reducing end of oligomeric/polymeric serogroup C capsular polysaccharide that carries a functional group attached to the anomeric carbon C2. R₃═OH, R₃=[→2)-α-Neu5Ac-(9→)ₓ]. R=FITC-lactose, R=FCHASE-lactose, R═N₃, R═F, R═(CH₂)ₓN₃ (for Figures A, B, and C)

FIG. 6: Schematic representation of wild-type and chimeric Neisseria meningitidis capsular polysaccharides. NmW-135: capsular polysaccharide of NmW-135 [→6)-α-D-Galp-(1→4)-α-Neu5Ac-(2→]ₙ, NmY: capsular polysaccharide of NmY [→6)-α-D-Glcp-(1→4)-α-Neu5Ac-(2→]ₙ, NmB/C: capsular polysaccharide of NmB [→8)-α-Neu5Ac-(2→]ₙ or of NmC [→9)-α-Neu5Ac-(2→]ₙ, NmX: capsular polysaccharide of NmX [→4)-α-D-GlcpNAc-(1→OPO₃→]ₙ, NmA: capsular polysaccharide of NmA [→+4)-α-D-ManpNAc-(1→OPO₃→]ₙ. Chimeric CPS may contain one or more building blocks of the indicated CPS structures. The buildings blocks may be of variable length.

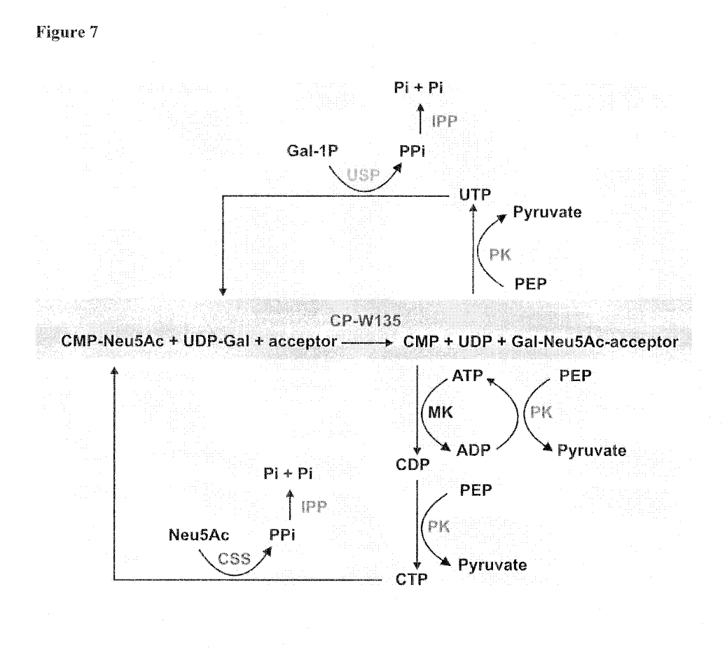

FIG. 7: CP-W135: Capsule polymerase NmW-135. MK: Myosin kinase (Sigma-Aldrich), PK: Pyruvate kinase (Sigma-Aldrich). CSS: CMP-Neu5Ac synthetase from NmB. IPP: Inorganic pyrophosphatase (Molecular Probes). USP: UDP-Sugar-Pyrophosphoryiase from Leishmania major (Damerow et al. J Biol Chem (2010), 285(2): 878-887). PEP: phosphoenolpyruvate. Gal-1P: galactose-1-phosphate.

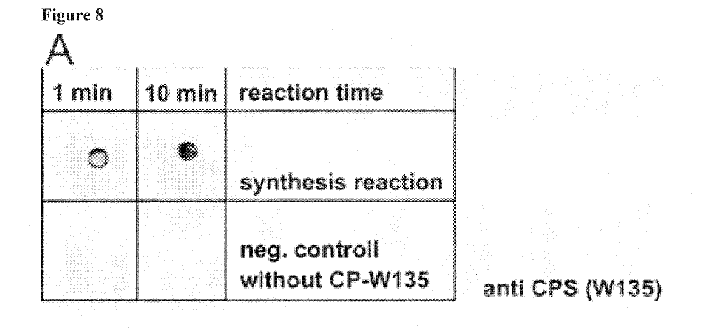

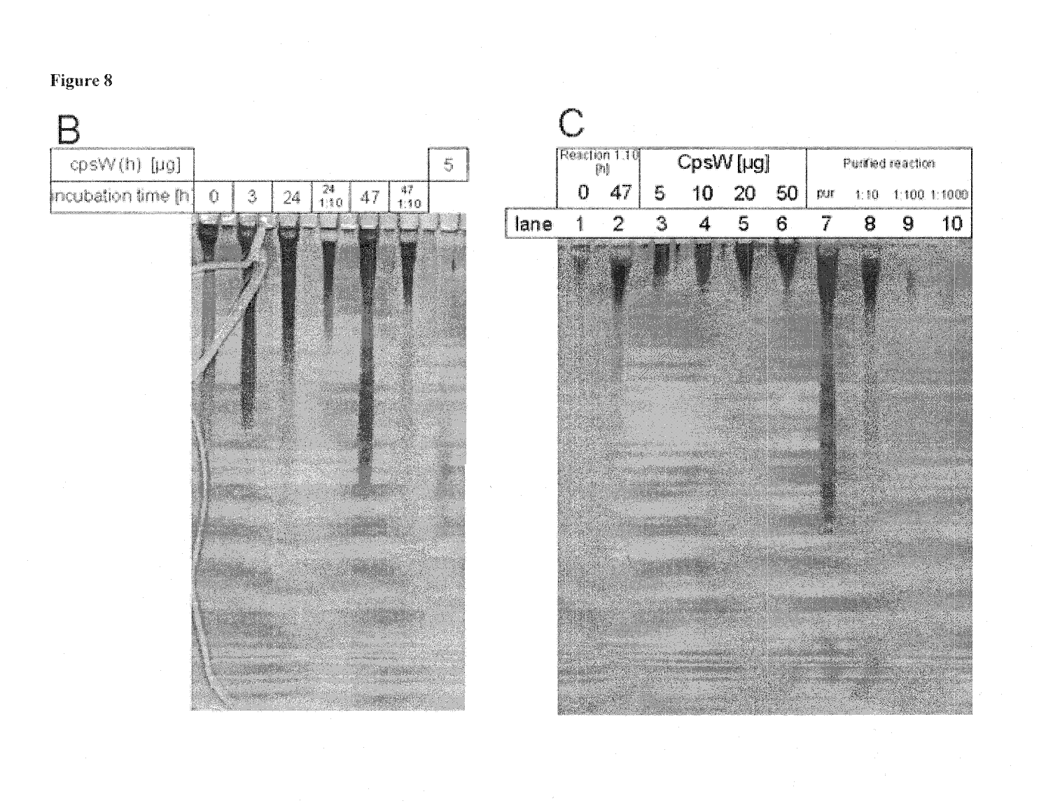

FIG. 8: In vitro synthesis of W-135 CPS from simple basic materials (galactose-1P, phosphoenolpyruvate and sialic acid) in a one-pot/six enzyme reaction. Product formation of the double cyclic reaction was analysed by A) Dot-blot analysis using the anti-W-135 CPS specific antibody mAb MNW1-3. B and C) Polysaccharide PAGE analysis. B) Samples of the reaction were taken after indicated time steps (0 h, 3 h, 24 h, and 47 h) and applied to the gel after mixing 1:1 with 2 M sucrose. For increased resolution of single band1s, dilutions (1:10) have been applied as well. C) Dilution series of a W-135 CPS standard from 5 to 50 μg allows an estimation of polysaccharide product formed by the cyclic reaction (reaction 1:10 [h]) and after purification of the same (purified reaction). Comparing lane 2, 3 and 8 allows a rough estimation of the amount of loaded polysaccharide and therefore of formed product, which is approx. 2 mg/200 μL reaction volume. This corresponds to 80 to 90% of the theoretical maximal yield. All samples were separated by 25% PAGE and saccharide structures were detected in a subsequent Alcian blue/silver staining.

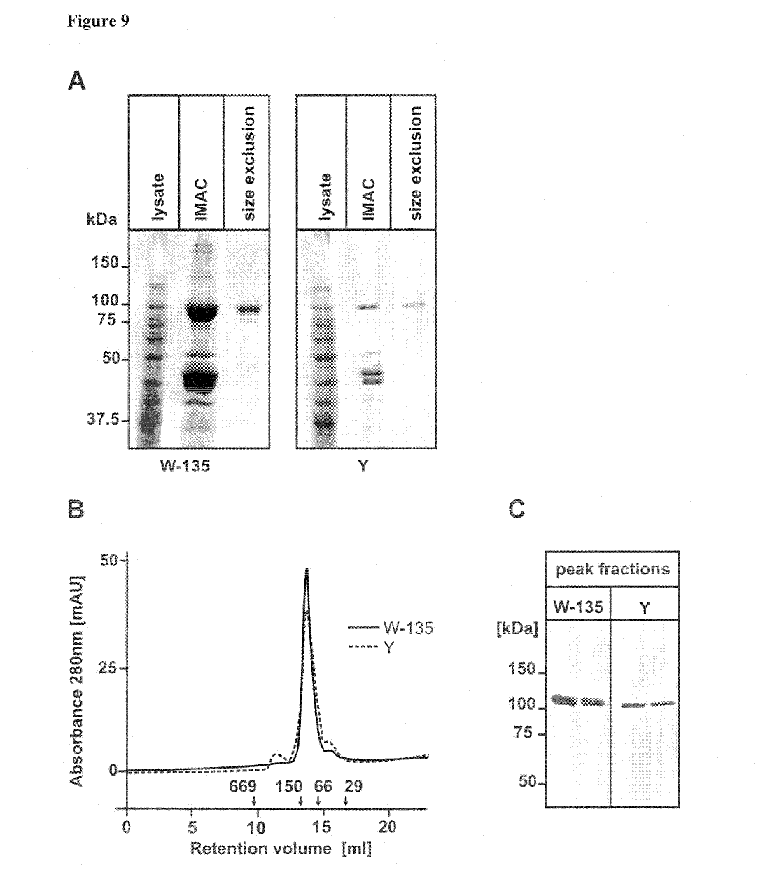

FIG. 9: Purification of recombinant CP-W135 and CP-Y. A) The C-terminally 6×His-tagged enzymes were expressed in E. coli and purified by IMAC and size exclusion chromatography in a two-step procedure. Protein fractions obtained throughout the purification were analyzed by Coomassule-stained SDS -PAGE (10%) as indicated; B-C) Oligomeric state of CP-W-135 and CP-Y. The quaternary structure of purified CP-W-135 and CP-Y was analyzed by size exclusion chromatography. Elution volumes of standard proteins are indicated by arrows (B), the main peak fraction was subsequently analyzed by Western Blot analysis directed against the 6×His-epitope tag (C).

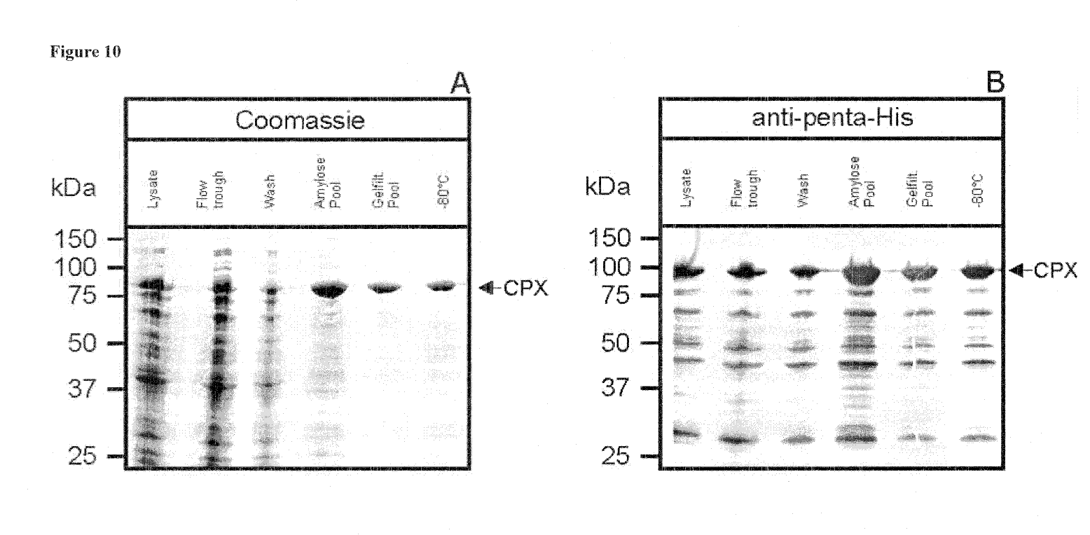

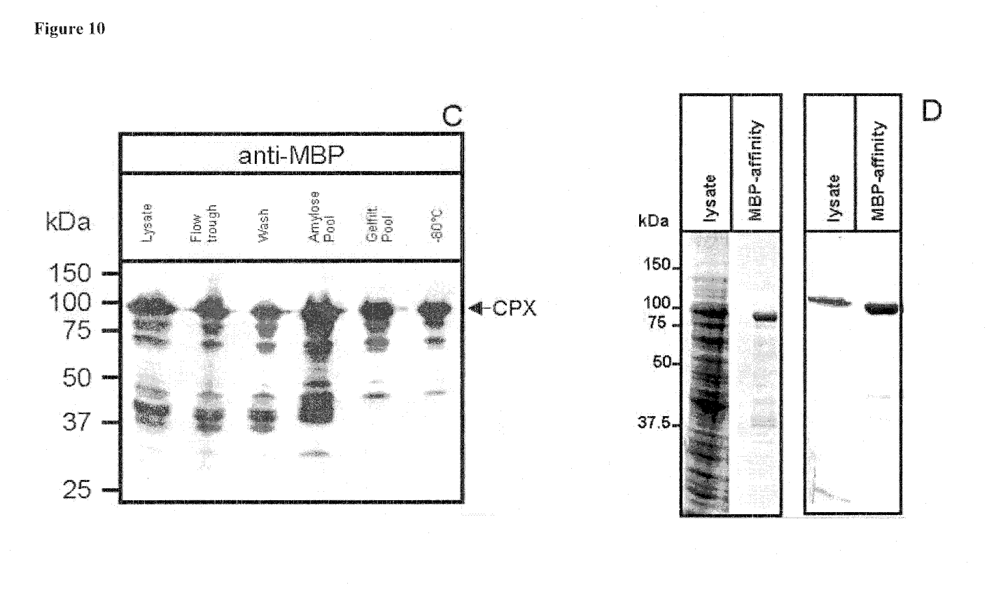

FIG. 10: Purification of recombinant CP-X. The C-terminally 6×His-tagged enzyme was N-terminally fused to MBP, expressed in E. coli and purified by MBP-affinity chromatography and size exclusion chromatography. Bacterial lysate, flowthrough, wash, pool of affinity chromatography, pool of gel filtration and −80° C. stored protein fractions were analysed by Coomassie stained SDS-Page (A) and by Western Blot analysis against the 6×His-tag (B) and MBP-tag (C) probed with anti-His mAb (anti-PentaHis, Qiagen) and anti MBP mAb HRP conjugated (NEB). (D) The C-terminally 6×His-tagged enzyme was N-terminally fused to MBP, expressed in E. coli and purified by MBP-affinity chromatography. Bacterial lysate and affinity purified protein fractions were analysed by Coomassie stained SDS-Page (left panel) and by Western Blot analysis (right panel) probed with anti-His mAb (anti-PentaHis, Qiagen).

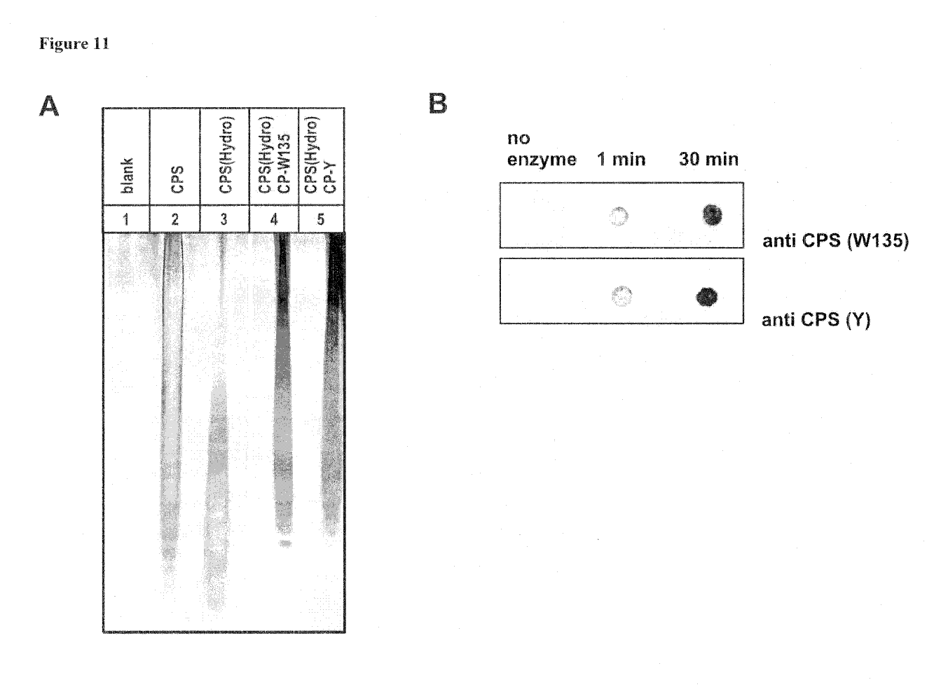

FIG. 11: In vitro synthesis of long serogroup W-135 and Y polymer chains. A) Polysaccharide PAGE analysis of CP-W-135 and CP-Y synthesis products. To obtain oligosaccharide acceptor substrates, purified serogroup W-135 CPS (lane 2) was hydrolysed (CPS_{Hydro}, lane 3) and subsequently used as primer material for in vitro polymerisation. Reaction mixtures contained the purified enzyme catalysts, the respective donor sugars CMP-Neu5Ac/UDP-Gal (lane 4) and CMP-Neu5Ac/UDP-Glc (lane 5) as well as the acceptor structure CPS_{Hydro}. All samples were separated by 25% PAGE and saccharide structures were detected in a subsequent Alcian blue/silver staining; B) immunostaining of the polysaccharides synthesized in A (lanes 4-5) using anti-CPS-W-135 (mAb MNW1-3) and anti-CPS-Y (mAb MNY4-1) specific antibodies. 5 μl aliquots of the reaction mixtures were dotted onto Hybond membranes after 1 min and min reaction time. As negative control, equivalent amounts of the acceptor structure CPS_{Hydro} were applied (no enzyme).

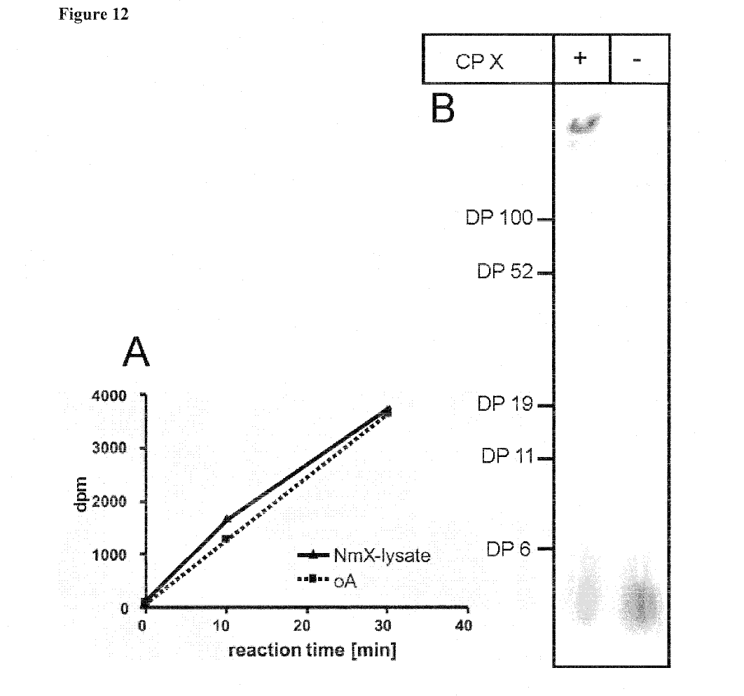

FIG. 12: In vitro synthesis of serogroup X CPS. A) Polymer synthesis was assayed in a radiochemical assay using purified CP-X as enzyme catalyst in the presence of UDP-[6-³H]-GcNAc (2 mCi/mmol, Perkin Elmer). Either no acceptor (oA) or whole VnAX-lysate was added. 5 μl aliquots were analysed after 0, 10 and 30 min reaction time. Samples were separated by descending paper chromatography and measured by scintillation counting. B) Additionally, radiolabelled reaction products were analysed by PAGE (25%). Samples with and without CP-X enzyme were incubated in the presence of radiolabelled donor sugar UDP-[6-³H]-GlcNAc (2 mCi/mmol, Perkin Elmer) and whole NmX-lysate.

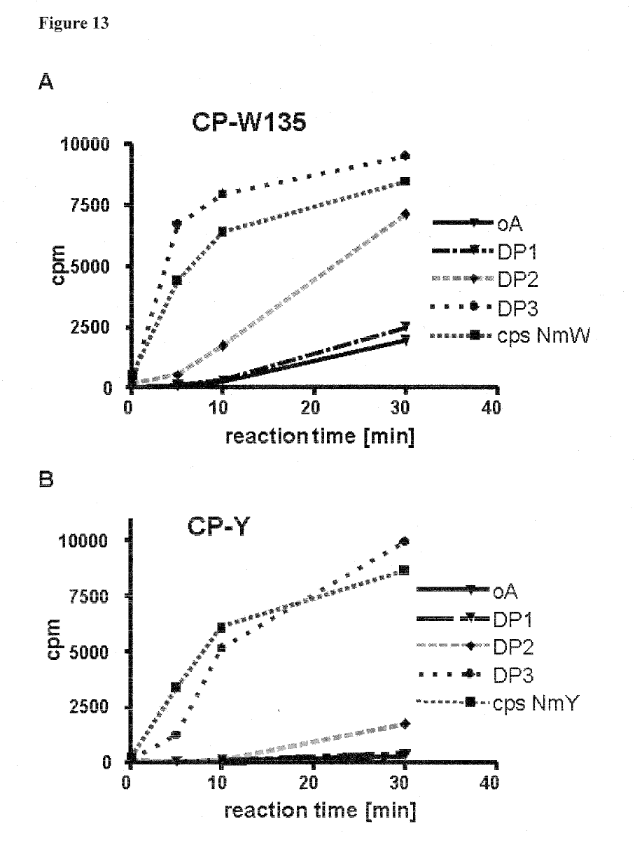

FIG. 13: Synthesis of serogroup W-135 and Y CPS starting from defined oligosaccharide acceptors. Purified CP-W-135 (A) and CP-Y (B) enzyme catalysts were used to elongate artificial acceptors. Polymer synthesis was assayed in a radiochemical assay in the presence of CMP-[¹⁴C]Neu5Ac. Reaction mixtures additionally contained the required UDP-hexose donor substrates (UDP-Gal for CP-W-135 and UDP-Glc for CP-Y) and artificial acceptor substrates as indicated. Samples were separated by descending paper chromatography and analyzed by scintillation counting. oA: no acceptor added, DP1: monomeric sialic acid, DP2: dimer of α2,8-linked sialic acid, DP3: trimer of α2,8-linked sialic acid, cps NmW: purified NmW-135 CPS, cps NmY: purified NmW-135 CPS.

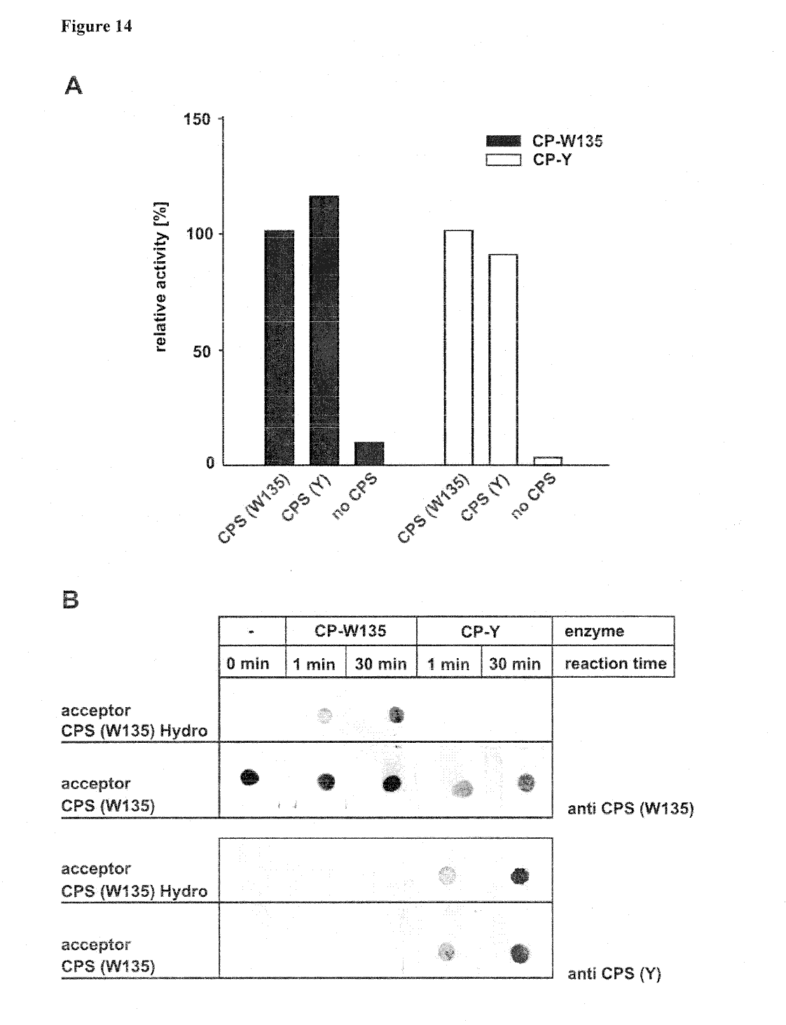

FIG. 14: In vitro synthesis of chimeric W135/Y-polymers. A) Product formation of purified CP-W-135 and CP-Y was analysed in the radiochemical assay as described in FIG. 13 in the presence of either W-135 or Y CPS compared to reactions without any CPS acceptor; B) In a parallel analysis recognition of the synthesized polysaccharides by CPS specific antibodies was analyzed to confirm the synthesis of dual-epitope CPS molecules. Either long-chain (CPS(W-135)) or hydrolysed (CPS(W-135)Hydro) fractions of purified serogroup W-135 CPS were used as primer material for in vitro CPS synthesis. 5 μl aliquots of the reactions were dotted onto Hybond membranes and bound CPS was subsequently detected by immunostaining unsing anti-CPS-W-135 (mAb MNW1-3, (25)) and anti-CPS-Y (mAb MNY4-1, (25)) specific antibodies followed by colour reaction.

FIG. 15: Schematic representation of Glc-1-P and possible derivatives thereof. A) glucose-1-phosphate; B) potential target-sites for derivatisations of Glc-1-P are represented by R₁, R₂, R₃ and R₄. Examples for R₁₋₄ are: R═H, R═OH, R═N₃, R═F, R═(CH₂)ₓN₃, R═COOH, R═(CH₂)ₓCOOH, R═NH(CO)CH₃, R═NH(CO)(CH₂)ₓCH₃, R═O(CO)CH₃, R═O(CO)(CH₂)ₓCH₃.

FIG. 16: Schematic representation of GlcNAc-1-P and possible derivatives thereof. A) N-Acetylglucosamine-1-phosphate; B) potential target-sites for derivatisations of GlcNAc-1-P are represented by R₁, R₂, R₃ and R₄. Examples R₁₋₄ are: R═H, R═OH, R═N₃, R═F, R═(CH₂)ₓN₃, R═COOH, R═(CH₂)ₓCOOH R═NH(CO)CH₃, R═N(CO)(CH₂)ₓCH₃, R═O(CO)CH₃, R═O(CO)(CH₂)ₓCH₃.

FIG. 17: Schematic representation of UDP-ManNAc and possible derivatives thereof. A) UDP-N-Acetylmannosamine; B) potential target-sites for derivatisations of UDP-ManNAc are represented by R₁, R₂, R₃ and R₄. Examples R₁₋₄ are: R═H, R═OH, R═N₃, R═F, R═(CH₂)ₓN₃, R═COOH, R═(CH₂)ₓCOOH, R═NH(CO)CH₃, R═NH(CO)(CH₂)ₓCH₃, R═O(CO)CH₃, R═O(CO)(CH₂)ₓCH₃.

FIG. 18: Schematic representation of ManNAc-1-P and possible derivatives thereof. A) N-Acetylmannosamine-1-phosphate; B) potential target-sites for derivatisations of ManNAc-1-P are represented by R₁, R₂, R₃ and R₄. Examples R₁₋₄ are: R═H, R═OH, R═N₃, R═F, R═(CH₂)ₓN₃, R═COOH, R═(CH₂)ₓCOOH, R═NH(CO)CH₃, R═NH(CO)(CH₂)ₓCH₃, R═O(CO)CH₃, R═O(CO)(CH₂)ₓCH₃.

FIG. 19: ELISA based assay to substantiate the formation of chimeric capsular polysaccharide B/W-135 CPS and B/Y CPS.
A) Control samples: DP50 (chain length of 50 units composed of α-2,8 linked polySia) W-135 CPS (capsular polysaccharide of NmW-135 harvested from bacteria) W-135 CPS hyd (hydrolyzed capsular polysaccharide of NmW-135 (W-135 CPS) harvested from bacteria) Samples: Reactions were carried out in the presence (+) and absence (−) of polymerase NmW-135 (CP-W-135) and DP50 to prove the formation of chimeric CPS. Samples are done in duplicates. B) Control samples: DP50 (chain length of 50 units composed of α-2,8 linked polySia) Y CPS (capsular polysaccharide of NmY harvested from bacteria) Samples: Reactions were carried out in the presence (+) and absence (−) of Polymerase NmY (CP-Y) and DP50 to prove the formation of chimeric CPS. Samples are done in duplicates.

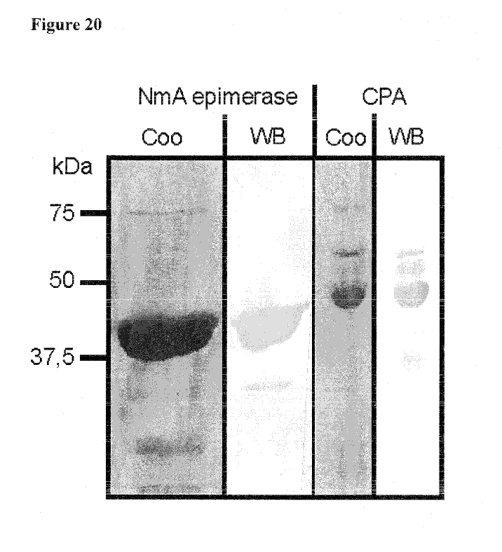

FIG. 20: Purification of recombinant UDP-GlcNAc epimerase and CP-A.
Purification of the capsular polymerase (CP-A) as well as the NmA UDP-GlcNAc epimerase (NmA epimerase). Both enzymes are expressed and purified as fusion constructs with an N-terminal Strep and a C-terminal hexa-histidine tag. The enzymes were purified by IMAC and protein fractions were analysed by Coomassie stained SDS-Page (COO) and by Western Blot (WB) analysis probed with anti-His mAb (anti-PentaHis, Qiagen).

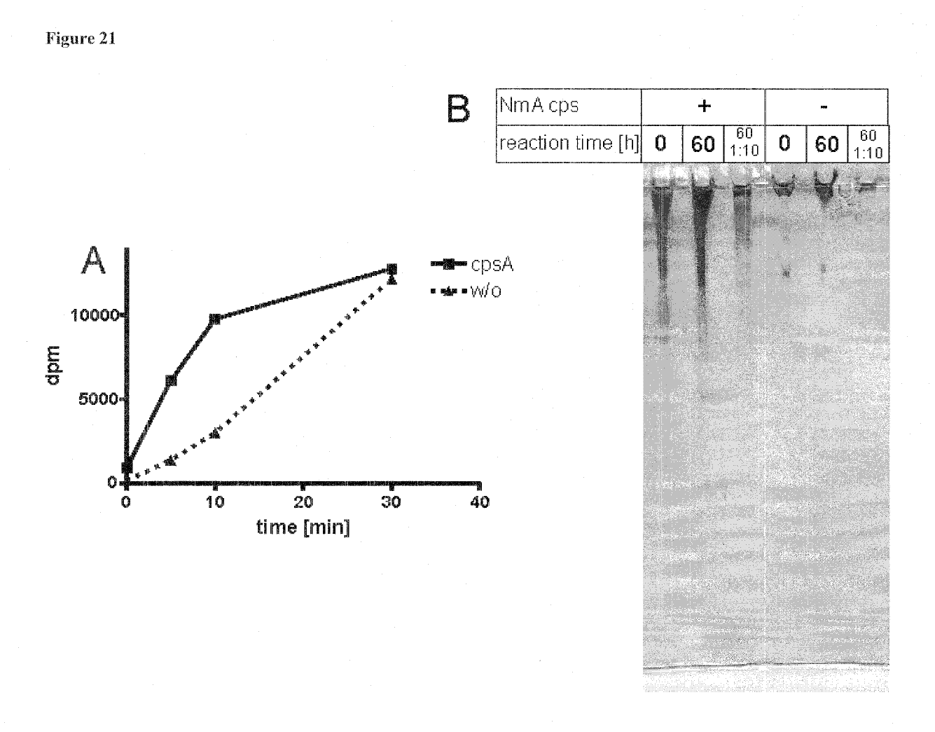

FIG. 21: In vitro synthesis of serogroup A CPS.
A) Polymer synthesis was assayed in a radiochemical assay using purified CP-A and purified UDP-GlcNAc epimerase as enzyme catalyst in the presence of UDP-[¹⁴C]-GlcNAc. Either no acceptor (w/o) or A CPS harvested from bacterial cells was added. 5 μl aliquots were analysed after 0, 10 and 30 min reaction time. Samples were separated by descending paper chromatography and measured by scintillation counting. B) Reaction samples after 0 min and 60 min of incubation time were applied to PAGE and developed by alcian blue silver-staining. Reactions containing capsular polysaccharide from NmA (A CPS) or not were carried out, showing that the polymerase is able to work without acceptor (de novo).

The Examples illustrate the invention.

### EXAMPLE 1

### Plasmids

CP-W-135 enzyme (capsule polymerase W-135) and CP-Y enzyme (capsule polymerase Y) were amplified by PCR from plasmids pHC4 and pHC5 (Claus et al., Molecular divergence of the sia locus in different serogroups of Neisseria meningitidis expressing polysialic acid capsules, Mol Gen Genet (1997), 257(1): 28-34), respectively, using oligonucleotides KS272 (GC GGA TCC GCT GTT ATT ATA TTT GTT AACG) and KS273 (CCG CTC GAG TTT TTC TTG GCC AAAAAA CTG). PCR products were ligated between BamHI and XhoI sites of the expression vector pET22b-Strep derived from pET-22b (Novagen) (Schwarzer et al., Characterization of a novel intramolecular chaperone domain conserved in endosialidases and other bacteriophage tail spike and fiber proteins, J Biol Chem (2007), 282(5): 2821-2831). The resulting constructs (pET22b-Strep-NmW135 and pET22b-Strep-NmY) carried an N-terminal Strep-tag II followed by a thrombin cleavage site and a C-terminal His-6-tag. The sequence identity of all constructs was confirmed by sequencing. Expression constructs lacking the N-terminal Strep-II-Tag were amplified from pHC4 and pHC5 (Claus et al., Molecular divergence of the sia locus in different serogroups of Neisseria meningitidis expressing polysialic acid capsules, Mol Gen Genet (1997), 257(1): 28-34) using the oligonucleotides KS422 (GC ATCT CAT ATG GCT GTT ATT ATA TTT GTT AAC G) and KS273 (CCG CTC GAG TTT TTC TTG GCC AAA AAA CTG). The PCR products were ligated between NdeI and XhoI sites of the expression vector pET22b (Novagen).

The CP-X enzyme (capsule polymerase X) was amplified by PCR from genomic serogroup X neisserial DNA using primer pairs KS423 (GC GGA TCC ATT AT AGC AAA ATT AGC AAA TTG) and KS424 (CCG CTC GAG TTG TCC ACT AGG CTG TGA TG). The PCR product was ligated between BamHI and XhoI sites of the expression vector pMBP-Strep-NmB-polyST (Freiberger et al., Biochemical characterization of a Neisseria meningitidis polysialyltransferase reveals novel functional motifs in bacterial sialyltransferases, Mol Microbiol (2007), 65(5): 1258-1275), resulting in the plasmid pMBP-XcbA-His.

Additionally, CP-A (capsule polymerase A) was amplified by PCR from genomic serogroup A neisserial DNA using primer pairs AB20 (GCA GAT CTT TTA TAC TTA ATA ACA GAA AAT GGC) and AB21 (CCG CTC GAG TTT CTC AAA TGA TGA TGG TAA TG). PCR product was ligated between BamHI and XhoI site of the expression vector pET22b-Strep derived from pET-22b (Novagen) (Schwarzer et al., J Biol Chem (2007), 282(5): 2821-2831). The resulting construct (pET22b-Strep-NmA) carried an N-terminal Strep-tag II followed by a thrombin cleavage site and a C-terminal His-6-tag. The sequence identity was confirmed by sequencing.

The UDP-GlcNAc-UDP-ManNAc epimerising enzyme (NmA-epimerase) was amplified by PCR from genomic serogroup A neisserial DNA using primer pairs AB22 (GCG GAT CCA AAG TCT TAA CCG TCT TTG) and AB233 (CCG CTC GAG TCT ATT CTT TAA TAA AGT TTC TAC A). PCR product was ligated between BamHI and iho, site of the expression vector pET22b-Strep derived from pET-22b (Novagen) (Schwarzer et al., J Biol Chem (2007), 282(5): 2821-2831). The resulting construct (pET22b-Strep-NmA epimerase) carried an N-terminal Strep-tag II followed by a thrombin cleavage site and a C-terminal His-6-tag. The sequence identity was confirmed by sequencing.

### EXAMPLE 2

### Expression and Purification of CP-W-135 and CP-Y Enzymes

Freshly transformed E. coli BL21 (DE3) (transformed with pET22b-Strep-NmW135 or pET22b-Strep-NmY) were grown at 15° C. and 225 rpm in auto-inducing ZYM-5052 medium (Studier, Protein production by auto-induction in high density shaking cultures, Protein Expr Purif (2005), 41(1): 207-234) containing 100 μg/ml carbenicillin. Cells were harvested after 78 h (6000×g, 15 min, 4° C.), washed once with PBS and stored at −20° C. Bacterial pellets from 250 ml of cultures were re-suspended in binding buffer (50 mM Tris/HCl pH 8.0, 3 mM NaCl) supplemented with protease inhibitors (40 mg/ml Bestatin, 1 μg/ml Pepstatin and 1 mM PMSF) to give a final volume of 15 ml. Cells were disrupted by sonication and samples were centrifuged (16000×g; 30 min, 4° C.). Lysates were filtered (Sartorius Minisart 0.8 μm) and recombinant proteins were bound to 1 ml HisTrap affinity columns (GE Healthcare). After washing with 10 column volumes of washing buffer (50 mM Tris/HCl, pH 8.0, 300 mM NaCl, 50 mM imidazole) bound proteins were eluted (50 mM Tris/HCl pH 8.0, 300 mM NaCl, 150 mM imidazole). Fractions containing the recombinant proteins were pooled, filtered (Millipore Ultrafree MC 0.2 μm) and applied to a Superdex 200 10/300 GL column (GE Healthcare) for further purification by size exclusion chromatography. Proteins were eluted at a flowrate of 0.5 ml/min with 50 mM Tris/HCl, pH 8.0, 300 mM NaCl, 2 mM DTT. Obtained protein samples were concentrated to 2 mg/ml using Amicon Ultra centrifugal devices (Millipore; 50 KDa MWCO), flash-frozen in liquid nitrogen and stored at −80° C. Results are shown in FIG. 9. The nucleotide sequence of capsule polymerase cloned from Neisseria meningitidis serogroup W-135 carrying an N-terminal StrepII and a C-terminal 6×His-tag is shown in SEQ ID NO: 13, the corresponding polypeptide sequence is shown in SEQ ID NO: 14. The nucleotide sequence of capsule polymerase cloned from Neisseria meningitidis serogroup Y carrying an N-terminal StrepII and a C-terminal 6×His-tag is shown in SEQ ID NO: 15, the corresponding polypeptide sequence is shown in SEQ ID NO: 16. The nucleotide sequence of capsule polymerase cloned from Neisseria meningitidis serogroup W-135 carrying a C-terminal 6×His-tag is shown in SEQ ID NO: 17, the corresponding polypeptide sequence is shown in SEQ ID NO: 18.

### EXAMPLE 3A

### Expression and Purification of CP-X Enzyme

Freshly transformed E. coli BL21 (DE3) (pMBP-XcbA-His) were grown at 15° C. and 225 rpm in auto-inducing ZYM-5052 medium containing 100 μg/ml carbenicillin (Studier, Protein production by auto-induction in high density shaking cultures, Protein Expr Purif (2005), 41(1): 207-234). Cells were harvested after 78 h (6000×g, 15 min, 4° C.), washed once with PBS and stored at −20° C. Bacterial pellets from 50 ml of cultures were re-suspended in 5 ml of binding buffer (20 mM Tris/HCl pH 7.5, 200 mM NaCl, 1 mM DTT) supplemented with protease inhibitors (40 mg/ml Bestatin, 1 μg/ml Pepstatin and 1 mM PMSF). Cells were disrupted by sonication and samples were centrifuged (16000×g; 30 min, 4° C.). Lysates were filtered (Sartorius Minisart 0.8 m) and recombinant proteins were bound to 1 ml amylose resin (New England Biolabs) for 1 h at room temperature. After washing with 10 column volumes of binding buffer (20 mM Tris/HCl pH 7.5, 200 mM NaCl, 1 mM DTT) bound proteins were eluted (20 mM Tris/HCl pH 7.5, 200 mM NaCl, 1 mM DTT, 2 mM maltose). Fractions containing the recombinant protein were pooled, concentrated to 2 mg/ml using Amicon Ultra centrifugal devices (Millipore; 50 KDa MWCO), flash-frozen in liquid nitrogen and stored at −80° C. Results are shown in FIG. 10D. The nucleotide sequence of capsule polymerase cloned from Neisseria meningitidis serogroup X carrying an N-terminal MBP and a C-terminal 6×His-tag is shown in SEQ ID NO: 19, the corresponding polypeptide sequence is shown in SEQ ID NO: 20.

### EXAMPLE 3B

### Extended Purification of CP-X Enzyme by Affinity Chromatography and Size Exclusion Chromatography

The CP-X enzyme was expressed and stored as already described in example 3. Bacterial pellets from 50 ml of cultures were re-suspended in 5 ml of binding buffer (20 mM Tris/HCl pH 7.5, 200 mM NaCl, 1 mM DTT) supplemented with protease inhibitors (40 mg/ml Bestatin, 1 μg/ml Pepstatin and 1 mM PMSF). Cells were disrupted by sonication and samples were centrifuged (16000×g; 30 ml, 4° C.). Lysates were filtered (Sartorius Minisart 0.8 μm) and recombinant proteins were bound to 1 ml amylose resin (New England Biolabs) for 1 h at room temperature. After washing with 10 column volumes of binding buffer (20 mM Tris/HCl pH 7.5, 200 mM NaCl, 1 mM DTT) bound proteins were eluted (20 mM Tris/HCl pH 7.5, 200 mM NaCl, 1 mM DTT, 10 mM maltose). Subsequently recombinant protein containing fractions were pooled and applied to a Superdex 200 10/300 GL column for further purification by size exclusion chromatography. Elution was done at a flowrate of 1 ml/min with 20 mM Tris, pH 7.5. Fractions containing the recombinant protein were pooled, concentrated to 2 mg/ml using Amicon Ultra centrifugal devices (Millipore; 50 KDa MWCO), flash-frozen in liquid nitrogen and stored at −80° C. Samples were taken throughout the purification and results are shown in FIG. 10A-C. The nucleotide sequence of capsule polymerase cloned from Neisseria meningitidis serogroup X carrying an N-terminal MBP and a C-terminal 6×His-tag is shown in SEQ ID NO: 19, the corresponding polypeptide sequence is shown in SEQ ID NO: 20.

### EXAMPLE 4

### Enzymatic In Vitro Synthesis of Serogroup WV-135 and Serogroup Y CPS

The purified enzyme catalysts (5-15 μg) were assayed in reaction buffer (20 mM Tris/HCl pH 8.0, 10 mM MgCl₂, 1 mM DTT) in the presence of 1 mM CMP-Neu5Ac (GERBU), 2 mM of either UDP-Gal (CP-W-135) or UDP-Glc (CP-Y) and hydrolysed W-135 CPS (0.16 μg/μl) as oligosaccharide acceptor structure in a total volume of 37.5 μl. Samples were incubated at room temperature and reactions were stopped at appropriate time intervals by addition of 1M sucrose.

The synthesized products were separated by PAGE (25%) and stained using a combined Alcian blue/silver staining procedure to prove in vitro synthesis of long CPS chains as described in (Bergfeld et al., The polysialic acid-specific O-acetyltransferase OatC from Neisseria meningitidis serogroup C evolved apart from other bacterial sialate O-acetyltransferases, J Biol Chem (2009), 284(1): 6-16). Briefly, samples were diluted with one volume of loading buffer (1 M sucrose) prior to loading on 25% Polyacrylamide gels (89 mM Tris, 89 mM boric acid, 2 mM EDTA, 25% Polyacrylamide). Additionally a mix of standard dyes with defined molecular size was applied (0.05% trypan blue, 0.02% Xylene cyanol, bromphenol blue, bromcrescole purple, phenol red) and the samples were electrophoresed (4° C., 23 V/cm) until the phenol red band reached the end of the gel. The gels were subsequently fixed for 1 h (40% EtOH, 5% acetic acid) and stained with 0.5% Alcian blue for 30 min. Prior to the 5 min oxidizing step (0.7% periodic acid, 40% ethanol, 5% acetic acid), background staining was removed with water. Following oxidation, gels were washed three times with water, incubated in silver stain (0.6% silver nitrate, 20 mM NaOH, 0.4% NH₄OH) for 10 min and again washed with water three times. Finally gels were incubated in developer (0.05% formaldehyde, 240 μM citric acid) until the polysaccharide bands were clearly visible. The development reaction was stopped by incubation in 5% acetic acid solution.

In a parallel analysis, recognition of the synthesized polysaccharides by CPS specific antibodies was analyzed. Prior to sucrose addition, 5 μl aliquots of the reactions were dotted onto Hybond XL-membranes. Membranes were dried and blocked in dry-milk (2% in PBS). Bound CPS was detected by immunostaining using anti-CPS-W-135 (mAb MNW1-3, (Longworth et al., O-Acetylation status of the capsular polysaccharides of serogroup Y and W135 meningococci isolated in the UK, FEMS Immunol Med Microbiol (2002), 32(2): 119-123) and anti-CPS-Y (mAb MNY4-1, (Longworth et al., O-Acetylation status of the capsular polysaccharides of serogroup Y and W135 meningococci isolated in the UK, FEMS Immunol Med Microbiol (2002), 32(2): 119-123) specific antibodies followed by colour reaction. For quantification by infrared fluorescence detection, membranes were blocked in Odyssey blocking buffer (LI-COR) and goat-anti-mouse IR680 (LI-COR) was used as secondary antibody (50 ng/ml in blocking buffer). Bound CPS was subsequently quantified according to the recommendations of the Odyssey infrared imaging system (L-COR). Results are shown in FIG. 11.

### EXAMPLE 5

### Enzymatic In Vitro Synthesis of Serogroup X CPS

The purified CP-X enzyme (5 μg) was assayed in reaction buffer (20 mM Tris/HCl pH 8.0, 20 mM MgCl₂, 2 mM DTT) containing 4 mM tritium labelled UDP-[6-³H]-GlcNAc (2 mCi/mmol, Perkin Elmer) and either 2 μl of whole NmX bacterial lysate or no further acceptor in a total volume of 24 μl. Samples were incubated at 37° C. and reactions were stopped at appropriate time intervals by mixing 5 μl aliquots of the reaction solution with 5 μl of chilled ethanol (96%). Samples were spotted on Whatman 3mM CHR paper and the chromatographically immobile tritium-labelled reaction products were quantified by scintillation counting following descending paper chromatography in 96% ethanol/1M ammonium acetate, pH 7.5 (7:3, v/v). Results are shown in FIG. 12A.

Additionally, CP-X was found to start polymer synthesis de novo. Moreover, samples were also applied to PAGE (25%) analysis after mixing 10 μl of the reaction with 10 μl of 2M Sucrose and electrophoresed at 400V for 3 h. To visualize [¹⁴C]-labelled reaction products, the gel was vacuum-dried immediately after electrophoreses and exposed to an imaging film (BioMax, Kodak). Results are shown in FIG. 12B.

### EXAMPLE 6

### Enzymatic In Vitro Synthesis of Serogroup W-135 and Serogroup Y Polysaccharides Starting from Defined Oligosaccharide Acceptors

To investigate the minimal acceptor substrate requirements of CP-W-135 and CP-Y, a small set of defined oligosaccharides was tested: Monomeric (DP1), dimeric (DP2) and trimeric (DP3) α2,8-linked sialic acid were obtained from Nacalai Tesque, W-135 CPS and Y CPS were a kind gift of U. Vogel, Würzburg. Both enzymes, CP-W-135 and CP-Y, could efficiently start polymer synthesis starting from the CPS acceptors and from the defined DP3 acceptor substrate. Moreover, CP-W-135 was also found to start polymer synthesis de novo.

Enzyme assays were performed as described (Vogel et al., Complement factor C3 deposition and serum resistance in isogenic capsule and lipooligosaccharide sialic acid mutants of serogroup B Neisseria meningitidis, Infect Immun 1997, 65(10): 4022-4029). Purified recombinant proteins (5-15 μg) were assayed in reaction buffer (20 mM Tris/HCl pH 8.0, 10 mM MgCl₂, 1 mM DTT) in the presence of 1 mM radiocarbon labeled CMP-[¹⁴C]Neu5Ac (0.13 mCi/mmol, GE Healthcare) and 2 mM of either UDP-Gal (for CP-W-135) or UDP-Glc (for CP-Y) (both carbohydrates from Sigma). Additionally 2 mM of (oligo)saccharide acceptor or 0.4 mg/ml of W-135 CPS or Y CPS were included in a total volume of 25 μl. Samples were incubated at room temperature and enzymatic activity was determined at appropriate time intervals by mixing 5 μl aliquots of the reaction solution with 5 μl of chilled ethanol (96%). Samples were spotted on Whatman 3MM CHR paper and the chromatographically immobile ¹⁴C-labelled reaction products were quantified by scintillation counting following descending paper chromatography in 96% ethanol/1M ammonium acetate, pH 7.5 (7:3, v/v). Results are shown in FIG. 13.

### EXAMPLE 7

### Enzymatic In Vitro Synthesis of Chimeric Neisserial Capsular Polysaccharides

To synthesize chimeric polysaccharides, the purified enzyme catalysts (5-15 μg) were incubated in reaction buffer (20 mM Tris/HCl pH 8.0, 10 mM MgCl₂, 1 mM DTT) in the presence of 1 mM CMP-Neu5Ac (GERBU), 2 mM of either UDP-Gal (CP-W-135) or UDP-Glc (CP-Y) and a CPS acceptor molecule (0.5-1 μg/μl) in a total volume of 37.5 μl. The following enzyme/acceptor pairs were used to synthesize the indicated chimeras in Table 1.

TABLE 1

| Chimera | Capsule polymerase | Acceptor |
|---|---|---|
| Y/W-135 | CP W-135 | CPS Y |
| W-135/Y | CP Y | CPS W-135 |
| B/W135 | CP W-135 | CPS B |
| B/Y | CP Y | CPS B |
| C/W-135 | CP W-135 | CPS C |
| C/Y | CP Y | CPS C |

### EXAMPLE 8

### Enzymatic CPS-W-135 Synthesis as One-Pot/Five Enzymes-Reaction

A double-cyclic reaction that continuously recycles the nucleotide sugar pools was designed. The basic materials for W-135 CPS synthesis are galactose-1P, phosphoenolpyruvate and sialic acid, whereas only catalytic amounts of the nucleotides are required. The reaction scheme is depicted in FIG. 7.

Purified CP-W-135 (30 μg) was assayed in reaction buffer (200 mM Tris/HCl pH 8.5, 20 mM MgCl₂, 2 mM DTT) containing 40 mM galactose-1-phosphate (GLYCON Biochemicals), 2 mM UTP, 1 mM CTP, 20 mM sialic acid (Neu5Ac, GERBU), 1 mM ATP, 100 mM phosphoenolpyruvate (Fluka), 3 μg CMP-Neu5Ac synthetase (Gilbert et al., Biotechnology Letters (1997), 19(5): 417-420), 3 U pyruvate kinase (Sigma), 1 U myosin kinase (Sigma), 4 μg UDP-sugar phosphorylase (Damerow et al., J Biol Chem (2010), 285(2): 878-887), 2 mM DP3 [Neu5Ac-α(2→8)-Neu5Ac-α(2→8)-Neu5Ac] and 6 mU inorganic phosphatase. Samples were incubated at 37° C. and 1 μl aliquots of the reaction were analyzed at appropriate time points by dot-blot analysis. The aliquots were dotted onto Hybond XL-membranes. Membranes were dried and blocked in dry-milk (2% in PBS). Bound CPS was detected by immunostaining using and anti-CPS-W-135 (mAb MNW1-3, Longworth et al., FEMS Immunol Med Microbiol (2002), 32(2): 119-123) specific antibody followed by colour reaction. For quantification by infrared fluorescence detection, membranes were blocked in Odyssey blocking buffer (LI-COR) and goat-anti-mouse IR680 (LI-COR) was used as secondary antibody (50 ng/ml in blocking buffer). Bound CPS was subsequently quantified according to the recommendations of the Odyssey infrared imaging system (LI-COR). Results are shown in FIG. 8A.

In an additional assay, purified CP-W-135 (30 μg) was assayed in reaction buffer (250 mM Tris/HC pH 8.0, 40 mM MgCl₂, 2 mM DTT) containing 40 mM galactose-1-phosphate (GLYCON Biochemicals), 2 mM UTP, 1 mM CTP, 20 mM sialic acid (Neu5Ac, GERBU), 1 mM ATP, 100 mM phosphoenolpyruvate (Fluka), 30 μg/ml CMP-Neu5Ac synthetase (Gilbert et al., Biotechnology Letters (1997), 19(5): 417-420), 6 U pyruvate kinase (Sigma), 2.5 U myosin kinase (Sigma), 30 μg/ml UDP-sugar phosphorylase, 2 mM DP3 [Neu5Ac-α(2→8)-Neu5Ac-α(2→8)-Neu5Ac] and 6 mU inorganic phosphatase.

Samples were analyzed by PAGE as exemplified in the following. For analysis and quantification by PAGE (25%), the samples were separated and stained using a combined Alcian blue/silver staining procedure to prove in vitro synthesis of long CPS chains as described in Bergfeld et al., J Biol Chem (2009), 284(1): 6-16. Briefly, samples were diluted with one volume of loading buffer (1 M sucrose) prior to loading on 25% Polyacrylamide gels (89 mM Tris, 89 mM boric acid, 2 mM EDTA, 25% Polyacrylamide). Additionally a mix of standard dyes with defined molecular size was applied (0.05% trypan blue, 0.02% Xylene cyanol, bromphenol blue, bromcrescole purple, phenol red) and the samples were electrophoresed (4° C., 23 V/cm) until the phenol red band reached the end of the gel. The gels were subsequently fixed for 1 h (40% EtOH, 5% acetic acid) and stained with 0.5% Alcian blue for 30 min. Prior to the 5 min oxidizing step (0.7% periodic acid, 40% ethanol, 5% acetic acid), background staining was removed with water. Following oxidation, gels were washed three times with water, incubated in silver stain (0.6% silver nitrate, 20 mM NaOH, 0.4% NH₄OH) for 10 min and again washed with water three times. Finally gels were incubated in developer (0.05% formaldehyde, 240 μM citric acid) until the polysaccharide bands were clearly visible. The development reaction was stopped by incubation in 5% acetic acid solution. Results are shown in FIGS. 8 B&C. For better estimation of the amount of formed product, a dilution series of serogroup W-135 CPS was included in a second PAGE. Results are shown in FIGS. 8B and C.

### EXAMPLE 9

### Cloning, Expression and Purification of His₆-Tagged Leishmania. major USP

The entire open reading frame of L. major UDP-sugar pyrophosphorylase (LmnjF17.1160) (Damerow et al., J Biol Chem (2010), 285)(2): 878-887) was amplified with the primer set ACL115 (CTG ACT CCA TAT GAC GAA CCC GTC CAA CTC C) and ACL116 (CTT AGC GGC CGC ATC AAC TTT GCC GGG TCA GCC G), containing integrated restriction sites for NdeI and NotI, respectively and inserted into a pET22b expression vector (Novagen), containing a C-terminal His₆-tag. For recombinant expression the vector was transformed into Ca²⁺-competent E. coli BL21(DE3) via heat shock. Cells were grown in Power Broth (AthenaES) at 37° C. to an OD of 1.0, transferred to 15° C. and the expression induced at 1.2 OD by addition of 1 mM isopropyl 1-thio-β-D-galactopyranoside. After 20 h the cells were harvested by centrifugation (6000×g, 15 min, 4° C.) and washed with phosphate-buffered saline.

A bacterial pellet obtained from 500 mL Power Broth solution was resuspended in 15 mL Ni²⁺-chelating buffer A_{Ni} (50 mM Tris/HCl pH 7.8, 300 mM NaCl) including protease inhibitors (40 μg/mL bestatin (Sigma), 4 g/mL pepstatin (Sigma), 0.5 g/mL leupeptin (Serva) and 1 mM phenylmethylsulfonyl fluoride (Roche Applied Science)). Cells were lysed by sonication with a microtip (Branson Sonifier, 50% duty cycle, output control 5, eight 30 s pulses for 8 min) and cell debris were removed by centrifugation (20.000×g, 15 min, 4° C.). The soluble fraction was loaded onto a 1 mL HisTrap HP Ni²⁺-chelating column (GE Healthcare). After a 20 mL wash with buffer A_{Ni} (50 mM Tris/HCl pH 8, 300 mM NaCl), the column was eluted with 20 mL buffer A_{Ni} containing 40 mM imidazole followed by a final elution step of 5 mL buffer A_{Ni} containing 300 mM imidazole. The fractions containing L. major USP were pooled and passed over a HiPrep 26/10 desalting column (GE Healthcare) to exchange buffer A_{Ni} to buffer A_{Q} (50 mM Tris/HCl pH 8.0). The sample was then loaded on a 1 mL Q-Sepharose FF anion exchange column (GE Healthcare) that was successively washed and eluted with 20 mL buffer A_{Q}, 20 mL buffer A_{Q} containing 100 mM NaCl and a final final volume of 5 mL buffer A_{Q} containing 300 mM NaCl. Again, the fractions containing the recombinant L. major USP were pooled and exchanged to standard buffer (Tris/HCl pH 7.8, 10 mM MgCl₂) via HiPrep 26/10 column. Purified samples were snap-frozen in liquid nitrogen and stored in standard buffer at −80° C.

Complementation of the E. coli DEV6 galU mutant strain was performed as previously described (Lamerz et al., J Biol Chem 2006, 281:16314-16322).

### EXAMPLE 10

### Size Exclusion Chromatography

Size exclusion chromatography on a Superdex 200 10/300 GL column (10×300 mm) (GE Healthcare) was used to determine the quaternary organization of the recombinant L. major USP (Damerow et al., J Biol Chem (2010), 285(2): 878-887). The column was equilibrated with 50 mL of standard buffer (50 mM Tris/HCl, pH 7.8, 10 mM MgCl₂, loaded with 100 μL of one of the following standard proteins, bovine carbonic anhydrase (3 mg/mL), bovine serum albumin (10 mg/mL), yeast alcohol dehydrogenase (1 mg/mL), potato β-amylase (4 mg/mL), and thyroglobulin (3 mg/mL) (protein standard kit; Sigma) or with purified recombinant His₆-tagged L. major USP (4 mg/mL) and eluted at a flow rate of 1 mL/min. The apparent molecular weight was determined by standard curve.

### EXAMPLE 11

### In Vitro Pyrophosphorylase Enzyme Assays

The formation of pyrophosphate in the forward reaction was detected with the EnzChek® Pyrophosphate Assay Kit (Molecular Probes). The assay medium contained 50 mM Tris/HCl pH 7.8, 10 mM MgCl₂, 1 mM DTT, 0.2 mM 2-amino-6-mercapto-7-methylpurine ribo-nucleoside (MESG), 0.03 units APP, 2.0 units PNP and varying amounts of sugar-1-phosphate and UTP ranging from 0.5 to 3 mM. Enzyme reactions were performed at 25° C. in a total volume of 100 μL and started by the addition of L. major USP (Damerow et al., J Biol Chem (2010), 285(2): 878-887). A control without USP was used for normalization.

UTP produced in the reverse reaction, was converted into one equivalent of inorganic phosphate by E. coli Cytidine Triphosphate (CTP)-synthase in presence of ATP, L-Gln and the cofactor GTP. Inorganic phosphate was then quantified using the EnzChek® Pyrophosphate Assay Kit (Molecular Probes) but omitting the first coupling enzyme. For these experiments, the CTP-synthase gene was recombinantly cloned from E. coli XL1-blue in a pET22b expression vector with a primer set including Nde I and Not I restriction sites (SD13: CTT ACA TAT GCA TCA TCA TCA TCA TCA CGC TAG CGG ATC CAT GAC AAC GAA CTA TAT TTT TGT GAC C, SD14: CTT AGC GGC CGC TTA CTT CGC CTG ACG TTT CTG G). The N-terminal His-tagged CTP-synthase was expressed and purified as described above for the USP, but without anion exchange chromatography. The assay mixture for the reverse reaction contained 50 mM Tris/HCl pH 7.8, 10 mM MgCl₂, 1 mM DTT, 0.2 mM MESG, 1 mM ATP, 1 mM L-Gln, 0.25 mM GTP, 3 μg CTP-synthase, 2.0 units PNP and 2 mM of UDP-sugar and pyrophosphate in a final volume of 100 μl. The reaction was initiated by addition of USP and normalized to buffer control.

Measurements were performed in 96-well half-area flat-bottom microplates (Greiner Bio-One) with the Power-WaveTM340 KC4 System (Bio-Tek). To exclude cross reactions all substrates and cofactors of coupling enzymes were tested against USP inhibition or competition and vice versa (data not shown). The determinations of K_{M} and Vₘₐₓ values were performed using varying substrate concentrations up to twelve triplicates, whereas the second substrate was set to a constant saturating concentration. The initial linear rates (y) were plotted against the substrate concentrations (x) and the Michaelis-Menten-kinetic was analysed in PRISM using nonlinear-regression (y=Vₘₐₓ·x/(K_{M}+x).

### EXAMPLE 12

### SDS-PAGE Analysis and Immunoblotting

SDS-PAGE was performed according to Laemmli (Laemmli, Nature 1970, 227: 680). Protein samples were separated on SDS-polyacrylamide gels composed of a 5% stacking gel and a 10% separating gel. Protein bands were visualized by Coomassie brilliant blue staining. For Western blot analysis, proteins were transferred to nitrocellulose membranes (Schleicher & Schüll GmbH). His₆-tagged proteins were detected using the penta-His antibody (Qiagen) at a concentration of 1 μg/mL and a goat anti-mouse Ig alkaline phosphatase-conjugate (Jackson ImmunoResearch).

### EXAMPLE 13

### STD-NMR

All STD NMR experiments were performed on a Bruker Avance DRX 600 MHz spectrometer equipped with a triple axis cryoprobe at 298 K in 50 mM deuterated TRIS buffer, pH 7.8 and 10 mM MgCl₂. The protein was saturated with a cascade of 40 selective Gaussian-shaped pulses of 50 ms duration with a 100 μs delay between each pulse resulting in a total saturation time of ˜2 s. The on- and off-resonance frequency was set to 0.7 ppm and ppm, respectively. In a typical STD NMR experiment, 0.5 M recombinant USP was used and all investigated ligands were added at a molecular ratio (protein/ligand) of 1:100. A total of 1024 scans per STD-NMR experiment were acquired, and a WATERGATE sequence was used to suppress the residual HDO signal. A spin lock filter with strength of 5 kHz and duration of 10 ms was applied to suppress protein background. Relative STD effects were calculated according to the equation A_{STD}=(I₀−Iₛₐₜ)/I₀=I_{STD}/I₀ by comparing the intensity of the signals in the STD-NMR spectrum (I_{STD}) with signal intensities of a reference spectrum (I₀). The STD signal with the highest intensity was set to 100%, and other STD signals were calculated accordingly (Mayer et al., Journal of the American Chemical Society 2001, 123:6108-6117).

### EXAMPLE 14

### Detection of Chimeric Capsular Polysaccharide Serogroup B/W-135 CPS

An ELISA-plate (Falcon REF: 353911 flexible) was precoated with 20 μl inactive Fndnsialidase (Schwarzer et., J B iol Chem (2009), 284(14): 9465-9474) 10 μg/ml in PBS for 90 min. Saturation of the plates surface was done by incubation of 175 μl 1% BSA for 16 h at 4° C. Reaction mixtures containing serogroup B CPS as at least one component of the chimeric CPS as described in example 7 were adsorbed at the surface of the plate at 25° C. for at least 1 h. After three consecutive steps of washing with PBS, wells were incubated with primary antibody mAb MNW1-3, (Longworth et al., FEMS Immunol Med Microbiol (2002), 32(2): 119-123) or mAb 735 (Frosch et al., Proc Natl Acad Sci USA (1985), 82(4): 1194-1198.) with 5 g/ml in 1% BSA/PBS for 1 h at 25° C. Detecting the (i) NmW-135 CPS (mAb MNW1-3) or (ii) NmB CPS (rmAb 735). For the development the secondary antibody, anti-mouse POX (SothernBiotech 1010-05) was used in recommended concentrations in a final volume of 20 μl/well in 1% BSA containing PBS for 80 min. After each antibody incubation three washing steps with PBS were applied. Development was done by applying ABTS (Roche) as described in its manual. The results of this assay are shown in FIG. 19A.

### EXAMPLE 15

### Detection of Chimeric Capsular Polysaccharide Serogroup B/Y CPS

An ELISA-plate (Falcon REF: 353911 flexible) was precoated with 20 μl inactive Endosialidase (Schwarzer et al., J Biol Chem (2009), 284(14): 9465-9474) 10 μg/ml in PBS for 90 min. Saturation of the plates surface was done by incubation of 175 μl 1% BSA for 16 h at 4° C. Reaction mixtures containing serogroup B CPS as at least one component of the chimeric CPS as described in example 7 were adsorbed at the surface of the plate at 25° C. for at least 1 h. After three consecutive steps of washing with PBS, wells were incubated with primary antibody mAb MNY4-1, (Longworth et al., FEMS Immunol Med Microbiol (2002), 32(2): 119-123) or mAb 735 (Frosch et al., Proc Natl Acad Sci USA (1985), 82(4): 1194-1198.) with 5 μg/ml in 1% BSA/PBS for 1 h at 25° C. Detecting the (i) NmY CPS (mAb MNY4-1) or (ii) NmB CPS (mAb 735). For the development the secondary antibody, anti-mouse POX (SothernBiotech 1010-05) was used in recommended concentrations in a final volume of 20 μl/well in 1% BSA containing PBS for 80 min. After each antibody incubation three washing steps with PBS were applied. Development was done by applying ABTS (Roche) as described in its manual. The results of this assay are shown in FIG. 19B.

### EXAMPLE 16

### Expression and Purification of CP-A and NmA-Epimerase

Freshly transformed E. coli BL21 (DE3) transformed with either pET22b-Strep-NmA or pET22b-Strep-NmA epimerase were grown at 15° C. and 225 rpm in PowerBroth (Athena) medium containing 100 μg/ml carbenicillin to an optical density OD600 of 1.8 before induction with 0.1 mM IPTG. Cells were harvested after 24 h (6000×g, 15 min, 4° C.), washed once with PBS and stored at −20° C. Bacterial pellets from 500 ml of cultures were re-suspended in binding buffer (50 mM Tris/HCl pH 8.0, 300 mM NaCl) supplemented with protease inhibitors (40 mg/ml Bestatin, 1 μg/ml Pepstatin and 1 mM PMSF) to give a final volume of 20 ml. Cells were disrupted by sonication and samples were centrifuged (16000×g; 30 min, 4° C.). Lysates were filtered (Sartorius Minisart 0.8 μm) and recombinant proteins were bound to 1 ml HisTrap affinity columns (GE Healthcare). After washing with 10 column volumes of washing buffer (50 mM Tris/HCl, pH 8.0, 300 mM NaCl, and 50 mM imidazole) bound proteins were eluted (50 mM Tris/HCl pH 8.0, 300 mM NaCl, 150 mM imidazole). Fractions containing the recombinant proteins were pooled, filtered (Millipore Ultrafree MC 0.2 μm) and applied to a Hi Prep 26/10 Desalting column (GE Healthcare) for further purification. Proteins were eluted at a flowrate of 1 ml/min with 50 mM Tris/HCl, pH 8.0, 50 mM NaCl. Obtained protein samples were concentrated to 6 mg/ml using Amicon Ultra centrifugal devices (Millipore; 30 KDa MWCO), flash-frozen in liquid nitrogen and stored at −80° C. Results are shown in FIG. 20. The nucleotide sequence of capsule polymerase cloned from Neisseria meningitidis serogroup A carrying an N-terminal StrepII and a C-terminal 6×His-tag is shown in SEQ ID NO: 21, the corresponding polypeptide sequence is shown in SEQ ID NO: 22. The nucleotide sequence of UDP-GlcNAc-epimerase cloned from Neisseria meningitidis serogroup A carrying an N-terminal StrepII and a C-terminal 6×His-tag is shown in SEQ ID NO: 23, the corresponding polypeptide sequence is shown in SEQ ID NO: 24.

### EXAMPLE 17

### Enzymatic In Vitro Synthesis of Serogroup a CPS

Purified CP-A (5 μg) and NmA UDP-GlcNAc epimerase (5 μg) were assayed in reaction buffer (50 mM Tris/HCl pH 8.0, 50 mM MgCl₂, 5 mM DTT) containing 2 mM [¹⁴C]labelled UDP-[¹⁴C]-GlcNAc, (Perkin Elmer) and either 3 μl of NmA capsular polysaccharide (a kind gift of U. Vogel, Wirzburg) or no further acceptor in a total volume of 25 μl. Samples were incubated at 37° C. and reactions were stopped at appropriate time intervals by mixing 5 μl aliquots of the reaction solution with 5 μl of chilled ethanol (96%). Samples were spotted on Whatman 3MM CHR paper and the chromatographically immobile radio-labelled reaction products were quantified by scintillation counting following descending paper chromatography in 96% ethanol/1M ammonium acetate, pH 7.5 (7:3, v/v). Results are shown in FIG. 21A. Moreover, CP-A was also found to start polymer synthesis de novo. Equal reactions were carried out with non radio-labelled substrates and reactions were stopped at appropriate time intervals by mixing 10 μl aliquots of the reaction solution with 10 μl 2M Sucrose. Samples were stored at −20° C. The samples were separated by PAGE (25°,%) and stained using a combined Alcian blue/silver staining procedure to prove in vitro synthesis of long CPS chains as described in (Bergfeld et al., J Biol Chem (2009), 284(1): 6-16). Briefly, samples were diluted with one volume of loading buffer (1 M sucrose) prior to loading on 25% Polyacrylamide gels (89 mM Tris, 89 mM boric acid, 2 mM EDTA, 25% Polyacrylamide). Additionally, a mix of standard dyes with defined molecular size was applied (0.05% trypan blue, 0.02% Xylene cyanol, bromphenol blue, bromcrescole purple, phenol red) and the samples were electrophoresed (4° C., 23 V/cm) until the phenol red band reached the end of the gel. The gels were subsequently fixed for 1 h (40% EtOH, 5% acetic acid) and stained with 0.5% Alcian blue for 30 min. Prior to the 5 min oxidizing step (0.7% periodic acid, 40% ethanol, 5% acetic acid), background staining was removed with water. Following oxidation, gels were washed three times with water, incubated in silver stain (0.6% silver nitrate, 20 mM NaOH, 0.4% NH₄OH) for 10 min and again washed with water three times. Finally gels were incubated in developer (0.05% formaldehyde, 240 μM citric acid) until the polysaccharide bands were clearly visible. The development reaction was stopped by incubation in 5% acetic acid solution. Results are shown in FIG. 21B. Again, CP-A was found to start polymer synthesis de novo.

### Sequences Referred to in the Specification:

[Capsule Polymerase Cloned from Neisseria meningitidis Serogroup W-135, Coding Sequence]

>CP_NmW135_cds(Y13970).seq
SEQ ID NO: 1
atggctgttattatatttgttaacggaattcgggctgtaaatggccttgttaaatcatctatcaatactgcaaac
gattttgctgaagaaggactggatgttcatttaattaattttgttggcaatattactggaggagagcatttatac
cccccattccacttacatcccaatgtcaaaacctccagcatcatagatttatttaatgacattccagaaaatgtt
agctgccgaaatactcctttttattctattcatcaacaattcttcaaagctgaatatagtgcccactataagcat
gttttgatgaaaattgaatctttattatctgcagaagatagcattatcttcactcatcctcttcaactggaaatg
tatcgtttagcgaataatgatatcaagtcaaaagccaaactaattgtacaaattcatggtaattatatggaagaa
atccataactatgaaattttggcacgaaatatcgattatgttgactatcttcaaacggtatctgatgaaatgctg
gaagaaatgcattcccatttcaaaatcaaaaaagacaaattagtttttattccaaacatcacttatcccatttca
ttagaaaaaaaagaagctgatttctttattaaggataatgaagacatcgataatgctcagaaatttaaacgtatc
tctattgttggcagcattcagccaagaaaaaaccaattggatgccattaaaatcatcaataaaattaaaaatgaa
aattacattttacagatatatggcaaatctattaataaagattactttgaattaattaaaaaatatattaaagac
aataagttacaaaaccgtatcttattcaaaggtgaatcttccgagcaggaaatttatgaaaatacagatatcctg
atcatgacatcagaaagtgagggatttccatatatatttatggaaggcatggtgtatgatattccaatcgttgta
tatgattttaaatatggagcgaatgattacagtaactataatgaaaatggttgtgtttttaaaactggtgatatt
tctggaatggcaaaaaaaataattgagctattaaataacccagaaaaatataaagaattagttcaatataatcac
aatcgcttcttaaaagaatatgcaaaagatgtggttatggctaaatatttcactattcttccgcgcagctttaat
aacgtatcattatcgtctgctttcagccgaaaagaattggacgaattccaaaatattactttttctattgaagat
tctaatgatttagctcatatttggaatttcgagctaaccaatcctgcacaaaatatgaatttttttgctttagtt
ggcaagcgaaaatttccaatggatgctcatatccaaggaacacagtgtacgattaagatagctcataaaaagaca
gggaatttattgtcgcttttactaaaaaaacgaaatcagttgaatttatcaaggggatataccttaattgcagaa
gataatagctatgaaaaatatattggagcaatatctaataaaggtaactttgaaattattgcaaataaaaagagc
tcattagttactataaacaaaagtaccttagagttgcatgagattccccatgaactacatcagaataaattactg
attgctttacccaacatgcaaacgcctctaaaaattactgatgataatttaatacctatccaagcctccataaaa
ttagaaaagattggaaatacttattacccatgtttcttgccatctggcatatttaataatatctgcttagattac
ggtgaagaatccaaaattattaattttagtaaatattcttataaatatatctatgactcaattcgtcatattgag
caacatacagatatatcggatattatcgtttgcaatgtttattcttgggaacttattcgtgcctcagttattgag
agccttatggaatttaccggaaaatgggaaaaacactttcagacttctcctaaaattgattatcgatttgatcat
gaaggtaagcgttcgatggatgatgtcttttcagaagaaacatttattatggaatttccgcgtaaaaatggtata
gataagaaaacagcagccttccaaaatataccaaacagtattgtaatggagtatccgcagaccaatggttacagt
atgcgcagtcattcactgaaaagtaatgtagttgcggcaaaacattttcttgaaaaattaaataaaattaaggta
gatattaaatttaaaaagcatgaccttacaaacatcaaaaaaatgaatcgaattatttatgagcatttaggcatt
aacataaatatcgaagcatttctaaaaccacgattagaaaaatttaagcgtgaagaaaaatattttcatgatttc
ttcaaaagaaataattttaaagaggtaatttttccaagcacttattggaatccaggtattatttgtgctgcacat
aaacaaggtattaaggtatctgatattcaatatgctgccattactccttatcatcctgcgtattttaaatcacca
aaatcacattacgttgctgataaattgttattatggtctgaatattggaatcatgagcttttaccaaatccaaca
cgagagattggttctggtgccgcatattggtatgcattagatgatgtgagattttcagaaaaactgaattatgac
tatatctttctatctcaaagtaggatttcttcgcgcttgcttagttttgcaattgagtttgcattaaaaaatcct
caactacagcttttattttctaagcatccagatgaaaatatagatttaaagaacagaattattcctgataatctt
ataatctccacggaatcttctatacaaggcatcaatgaatctcgcgttgctgtaggtgtttattcaactagctta
tttgaggcattagcatgcggcaaacaaacttttgttgttaaatatccgggatatgaaattatgtcaaatgaaata
gattcagggttattctttgcagtagaaacacctgaagaaatgcttgagaaaacaagcccgaattgggtggctgtg
gcagatattgaaaaccagttttttggccaagaaaaataa

[Capsule Polymerase Cloned from Neisseria meningitidis Serogroup W-135, Amino Acid Sequence]

>CP_NmW135_(Y13970).pro
SEQ ID NO: 2
MAVIIFVNGIRAVNGLVKSSINTANAFAEEGLDVHLINFVGNITGAEHLYPPFHLHPNVKTSSIIDLFNDIPENV
SCRNTPFYSIHQQFFKAEYSAHYKHVLMKIESLLSAEDSIIFTHPLQLEMYRLANNDIKSKAKLIVQIHGNYMEE
IHNYEILARNIDYVDYLQTVSDEMLEEMHSHFKIKKDKLVFIPNITYPISLEKKEADFFIKDNEDIDNAQKFKRI
SIVGSIQPRKNQLDAIKIINKIKNENYILQIYGKSINKDYFELIKKYIKDNKLQNRILFKGESSEQEIYENTDIL
IMTSESEGFPYIFMEGMVYDIPIVVYDFKYGANDYSNYNENGCVFKTGDISGMAKKIIELLNNPEKYKELVQYNH
NRFLKEYAKDVVMAKYFTILPRSFNNVSLSSAFSRKELDEFQNITFSIEDSNDLAHIWNFELTNPAQNMNFFALV
GKRKFPMDAHIQGTQCTIKIAHKKTGNLLSLLLKKRNQLNLSRGYTLIAEDNSYEKYIGAISNKGNFEIIANKKS
SLVTINKSTLELHEIPHELHQNKLLIALPNMQTPLKITDDNLIPIQASIKLEKIGNTYYPCFLPSGIFNNICLDY
GEESKIINFSKYSYKYIYDSIRHIEQHTDISDIIVCNVYSWELIRASVIESLMEFTGKWEKHFQTSPKIDYRFDH
EGKRSMDDVFSEETFIMEFPRKNGIDKKTAAFQNIPNSIVMEYPQTNGYSMRSHSLKSNVVAAKHFLEKLNKIKV
DIKFKKHDLANIKKMNRIIYEHLGININIEAFLKPRLEKFKREEKYFHDFFKRNNFKEVIFPSTYWNPGIICAAH
KQGIKVSDIQYAAITPYHPAYFKSPKSHYVADKLFLWSEYWNHELLPNPTREIGSGAAYWYALDDVRFSEKLNYD
YIFLSQSRISSRLLSFAIEFALKNPQLQLLFSKHPDENIDLKNRIIPDNLIISTESSIQGINESRVAVGVYSTSL
FEALACGKQTFVVKYPGYEIMSNEIDSGLFFAVETPEEMLEKTSPNWVAVADIENQFFGQEK

[Capsule Polymerase Cloned from Neisseria meningitidis Serogroup Y, Coding Sequence]

>CP_NmY_cds(Y13969).seq
SEQ ID NO: 3
atggctgttattatatttgttaacggaattcgggctgtaaatggccttgttaaatcatctatcaatactgcaaac
gcttttgctgaagaaggactggatgttcatttaattaattttgttggcaatattactggagcagagcatttatcc
cccccattccacttacatcccaatgtcaaaacctccagcatcatagatttatttaatgacattccagaaaatgtt
agctgccgaaatattcctttttattctatccatcaacaattcttcaaagccgaatacagtgcccactataagcat
gttttgatgaaaattgaatctttattatctgaagaagatagcattatcttcactcatcctcttcaactggaaatg
tatcgtttagcgaataataatattaagtcaaaagccaagctaattgtacaaattcatggtaactatatggaagaa
atccataactatgaaatttgggcacgaaatatcgattatgttgattatcttcaaacggtatctgatgaaatgctg
gaagaaatgcattcccatttcaaaatcaaaaaagacaaattagtttttattccaaacatcacttatcccatttca
ttagaaaaaaaagaagctgatttctttattaaggataataaagacattgataatgctcagaaatttaaacgtatc
tctattgttggcagtattcagccaagaaaaaaccaattggatgccattaaaatcatcaataaaattaaaaatgaa
aattacattttacagatatatggcaaatctattaataaagattactttgaattaattaaaaaatatattaaagac
aataagttacaaaaccgtatcttattcaaaggtgaatcttccgagcaggaaatttatgagaatacagatatccta
atcatgacatctcaaagcgaaggctttggttatatatttctagagggtatggtgtacgatatccctatccttgcc
tataattttaaatatggagcgaatgattttagcaattataatgaaaacgcttcagtttttaaaactggtgatatt
tctggaatggcaaaaaaaataattgagctattaaataacccagaaaaatataaagaattagttcaatataatcac
aatcgcttcttaaaagaatatgcaaaagatgtggttatggctaaatatttcactattcttccgcgcagctttaat
aacgtatcattatcgtctgctttcagccgaaaagaattggacgaattccaaaatattactttttctattgaagat
tctaatgatttagctcatatttggaatttcgagctaaccaatcctgcacaaaatatgaatttttttgctttagtt
ggcaagcgaaaatttccaatggatgctcatatccaaggaacacagtgtacgattaagatagctcataaaaagaca
gggaatttattgtcgcttttactaaaaaaacgaaatcagttgaatttatcaaggggatataccttaattgcagaa
gataatagctatgaaaaatatattggagcaatatctaataaaggtaactttgaaattattgcaaataaaaagaac
tcattagttactataaacaaaagtaccttagagttgcatgagattccccatgaactacatcagaataaattactg
attgctttacccaacatgcaaacgcctctaaaaattactgatgataatttaatacctatccaagcctccataaaa
ttagaaaagattggaaatacttattacccatgtttcttgccatctggcatatttaataatatctgcttagattac
ggtgaagaatccaaaattattaattttagtaaatattcttataaatatatctatgactcaattcgtcatattgag
caacatacagatatatcggatattatcgtttgcaatgtttattcttgggaacttattcgtgcctcagttattgag
agccttatggaatttaccggaaaatgggaaaaacactttcagacttctcctaaaattgattatcgatttgatcat
gaaggtaagcgttcgatggatgatgtcttttcagaagaaacatttattatggaatttccgcgtaaaaatggtata
gataagaaaacagcagccttccaaaatataccaaacagtattgtaatggagtatccgcagaccaatggttacagt
atgcgcagtcattcactgaaaagtaatgtagttgoggcaaaacattttcttgaaaaattaaataaaattaaggta
gatattaaatttaaaaagcatgaccttgcaaacatcaaaaaaatgaatcgaattatttatgagcatttaggcatt
aacataaatatcgaagcatttctaaaaccacgattagaaaaatttaagcgtgaagaaaaatattttcatgatttc
ttcaaaagaaataattttaaagaggtaatttttccaagcacttattggaatccaggtattatttgtgctgcacat
aaacaaggtattaaggtatctgatattcaatatgctgccattactccttatcatcctgcgtattttaaatcacca
aaatcacattacgttgctgataaattgttcttatggtctgaatattggaatcatgagcttttaccaaatccaaca
cgagagattggttctggtgccgcatattggtatgcattagatgatgtgagattttcagaaaaactgaattatgac
tatatctttctatctcaaagtaggatttcttcgcgcttgcttagttttgcaattgagtttgcattaaaaaatcct
caactacagcttttattttctaagcatctagatgaaaatatagatttaaagaacagaattattcctgataatctt
ataatctccacggaatcttctatacaaggcatcaatgaatctcgcgttgctgtaggtgtttattcaactagctta
tttgaggcattagcatgcggcaaacaaacttttgttgttaaatatccgggatatgaaattatgtcaaatgaaata
gattcagggttattctttgcagtagaaacacctgaagaaatgcttgagaaaacaagcccgaattgggtggctgtg
gcagatattgaaaaccagttttttggccaagaaaaataa

[Capsule Polymerase Cloned from Neisseria Meningitis Serogroup Y, Amino Acid Sequence]

>CP_NmY_(Y13969).pro
SEQ ID NO: 4
MAVIIFVNGIRAVNGLVKSSINTANAFAEEGLDVHLINFVGNITGAEHLSPPFHLHPNVKTSSIIDLFNDIPENV
SCRNIPFYSIHQQFFKAEYSAHYKHVLMKIESLLSEEDSIIFTHPLQLEMYRLANNNIKSKAKLIVQIHGNYMEE
IHNYEIWARNIDYVDYLQTVSDEMLEEMHSHFKIKKDKLVFIPNITYPISLEKKEADFFIKDNEDIDNAQKFKRI
SIVGSIQPRKNQLDAIKIINKIKNENYILQIYGKSINKDYFELIKKYIKDNKLQNRILFKGESSEQEIYENTDIL
IMTSQSEGFGYIFLEGMVYDIPILAYNFKYGANDFSNYNENASVFKTGDISGMAKKIIELLNNPEKYKELVQYNH
NRFLKEYAKDVVMAKYFTILPRSFNNVSLSSAFSRKELDEFQNITFSIEDSNDLAHIWNFELTNPAQNMNFFALV
GKRKFPMDAHIQGTQCTIKIAHKKTGNLLSLLLKKRNQLNLSRGYTLIAEDNSYEKYIGAISNKGNFEIIANKKN
SLVTINKSTLELHEIPHELHQNKLLIALPNMQTPLKITDDNLIPIQASIKLEKIGNTYYPCFLPSGIFNNICLDY
GEESKIINFSKYSYKYIYDSIRHIEQHTDISDIIVCNVYSWELIRASVIESLMEFTGKWEKHFQTSPKIDYRFDH
EGKRSMDDVFSEETFIMEFPRKNGIDKKTAAFQNIPNSIVMEYPQTNGYSMRSHSLKSNVVAAKHFLEKLNKIKV
DIKFKKHDLANIKKMNRIIYEHLGININIEAFLKPRLEKFKREEKYFHDFFKRNNFKEVIFPSTYWNPGIICAAH
KQGIKVSDIQYAAITPYHPAYFKSPKSHYVADKLFLWSEYWNHELLPNPTREIGSGAAYWYALDDVRFSEKLNYD
YIFLSQSRISSRLLSFAIEFALKNPQLQLLFSKHLDENIDLKNRIIPDNLIISTESSIQGINESRVAVGVYSTSL
FEALACGKQTFVVKYPGYEIMSNEIDSGLFFAVETPEEMLEKTSPNWVAVADIENQFFGQEK.

[Capsule Polymerase Cloned from Neisseria meningitidis Serogroup X, Coding Sequence]

>CP_NmX_cds(AAP44500).seq
SEQ ID NO: 5
atgattatgagcaaaattagcaaattggtaacccacccaaaccttttctttcgagattatttcttaaaaaaagca
ccgttaaattatggcgaaaatattaaacctttaccagtcgaaacctcttctcatagcaaaaaaaatacagcccat
aaaacacccgtatcatccgaccaaccaattgaagatccatacccagtaacatttccaattgatgtagtttatact
tgggtagattcagatgatgaaaaattcaatgaagaacgcctaaagtttcaaaattcaagcacatctgagactcta
caaggcaaagcagaaagcaccgatattgcaagattccaatcacgcgacgaattaaaatattcgattcgaagcctg
atgaagtatgccccatgggtaaatcatatttacattgtaacaaatggtcaaataccaaaatggttagataccaac
aatacaaaggtaacgattatccctcactcaactattatcgacagtcaatttctccctacttttaattctcacgtc
attgaatcctctctatataaaatcccaggattatcagagcattacatttatttcaatgatgatgtcatgctagct
agagatttaagcccatcttatttctttacaagcagcggattagcaaaactgtttattaccaactctcgtctacca
aatggctataagaatgtgaaagacacaccaacccaatgggcctcaaaaaattcccgtgagcttttacatgcagaa
acaggattttgggctgaagccatgtttgcacatacttttcatccacaacgtaaaagtgtacatgaatctattgaa
cacctatggcatgaacaattaaatgtttgtcgtcaaaaccgtttccgtgatatttcagatattaacatggcgaca
ttcctgcaccaccattttgccattttgacaggccaagctcttgctacacgcactaaatgtatttactttaacgtt
cgctctcctcaagcagctcagcattacaaaacattattagctcgaaaaggaagcgaatacagcccacattctatc
tgcttaaatgatcatacatcgagcaataaaaatattttatctaattacgaagccaaattacaaagctttttagaa
acatactatccagatgtatcagaagcagaaattctccttcctactaaatctgaagtagctgaattagttaaacat
aaagattatttaactgtatatactaaattattacctattatcaataagcagctggtcaataaatataataaacct
tattcatatcttttctattatttaggtttatctgcccggtttttatttgaagaaacgcaacaagaacactaccgg
gaaactgctgaagaaaatttacaaatcttttgtggcctaaacccaaaacatacactagccctcaaatacttagcg
gatgtcaccctcacatcacagcctagtggacaataa

[Capsule Polymerase Cloned from Neisseria meningitidis Serogroup X, Amino Acid Sequence]

>CP_NmX_(AAP44500).pro
SEQ ID NO: 6
MIMSKISKLVTHPNLFFRDYFLKKAPLNYGENIKPLPVETSSHSKKNTAHKTPVSSDQPIEDPYPVTFPIDVVYT
WVDSDDEKENEERLKFQNSSTSETLQGKAESTDIARFQSRDELKYSIRSLMKYAPWVNHIYIVTNGQIPKWLDTN
NTKVTIIPHSTIIDSQFLPTFNSHVIESSLYKIPGLSEHYIYFNDDVMLARDLSPSYFFTSSGLAKLFITNSRLP
NGYKNVKDTPTQWASKNSRELLHAETGFWAEAMFAHTFHPQRKSVHESIEHLWHEQLNVCRQNRFRDISDINMAT
FLHHHFAILTGQALATRTKCIYFNVRSPQAAQHYKTLLARKGSEYSPHSICLNDHTSSNKNILSNYEAKLQSFLE
TYYPDVSEAEILLPTKSEVAELVKHKDYLTVYTKLLPIINKQLVNKYNKPYSYLFYYLGLSARFLFEETQQEHYR
ETAEENLQIFCGLNPKHTLALKYLADVTLTSQPSGQ.

[Capsule Polymerase Cloned from Neisseria meningitidis Serogroup A, Coding Sequence]

>CP-NmA (NC_003116 REGION: 183321 . . . 184958)
SEQ ID NO: 7
atgtttatacttaataacagaaaatggcgtaaacttaaaagagaccctagcgctttctttcgagatagtaaattt
aactttttaagatatttttctgctaaaaaatttgcaaagaattttaaaaattcatcacatatccataaaactaat
ataagtaaagctcaatcaaatatttcttcaaccttaaaacaaaatcggaaacaagatatgttaattcctattaat
ttttttaattttgaatatatagttaaaaaacttaacaatcaaaacgcaataggtgtatatattcttccttctaat
cttactcttaagcctgcattatgtattctagaatcacataaagaagactttttaaataaatttcttcttactatt
tcctctgaaaatttaaagcttcaatacaaatttaatggacaaataaaaaatcctaagtccgtaaatgaaatttgg
acagatttatttagcattgctcatgttgacatgaaactcagcacagatagaactttaagttcatctatatctcaa
ttttggttcagattagagttctgtaaagaagataaggattttatcttatttcctacagctaacagatattctaga
aaactttggaagcactctattaaaaataatcaattatttaaagaaggcatacgaaactattcagaaatatcttca
ttaccctatgaagaagatcataattttgatattgatttagtatttacttgggtcaactcagaagataagaattgg
caagagttatataaaaaatataagcccgactttaatagcgatgcaaccagtacatcaagattccttagtagagat
gaattaaaattcgcattacgctcttgggaaatgaatggatccttcattcgaaaaatttttattgtctctaattgt
gctcccccagcatggctagatttaaataaccctaaaattcaatgggtatatcacgaagaaattatgccacaaagt
gcccttcctacttttagctcacatgctattgaaaccagcttgcaccatataccaggaattagtaactattttatt
tacagcaatgacgacttcctattaactaaaccattgaataaagacaatttcttctattcgaatggtattgcaaag
ttaagattagaagcatggggaaatgttaatggtgaatgtactgaaggagaacctgactacttaaatggtgctcgc
aatgcgaacactctcttagaaaaggaatttaaaaaatttactactaaactacatactcactcccctcaatccatg
agaactgatattttatttgagatggaaaaaaaatatccagaagagtttaatagaacactacataataaattccga
tctttagatgatattgcagtaacgggctatctctatcatcattatgccctactctctggacgagcactacaaagt
tctgacaagacggaacttgtacagcaaaatcatgatttcaaaaagaaactaaataatgtagtgaccttaactaaa
gaaaggaattttgacaaacttcctttgagcgtatgtatcaacgatggtgctgatagtcacttgaatgaagaatgg
aatgttcaagttattaagttcttagaaactcttttcccattaccatcatcatttgagaaataa

[Capsule Polymerase Cloned from Neisseria meningitidis Serogroup A, Amino Acid Sequence]

>CP-NmA (YP_002341743).pro
SEQ ID NO: 8
Mfilnnrkwrklkrdpsaffrdskfnflryfsakkfaknfknsshihktniskaqsnisstlkqnrkqdmlipin
ffnfeyivkklnnqnaigvyilpsnltlkpalcileshkedflnkflltissenlklqykfngqiknpksvneiw
tdlfsiahvdmklstdrtlsssisqfwfrlefckedkdfilfptanrysrklwkhsiknnqlfkegirnyseiss
lpyeedhnfdidlvftwvnsedknwqelykkykpdfnsdatstsrflsrdelkfalrswemngsfirkifivsnc
appawldlnnpkiqwvyheeimpqsalptfsshaietslhhipgisnyfiysnddflltkplnkdnffysngiak
lrleawgnvngectegepdylngarnantllekefkkfttklhthspqsmrtdilfemekkypeefnrtlhnkfr
slddiavtgylyhhyallsgralqssdktelvqqnhdfkkklnnvvtltkernfdklplsvcindgadshlneew
nvqvikfletlfplpssfek

[UDP-Sugar Phosphorylase Cloned from Leishmania major, Coding Sequence]

>Leishmania_USP.seq
SEQ ID NO: 9
ATGACGAACCCGTCCAACTCCAACCTGCAGGCCTTGCGCGAGGAGCTCTGCACGCCTGGCCTGGATCAGGGTCAC
CTCTTCGAGGGATGGCCGGAGACTGTGGATGAGTGCAACGAGAGGCAGATCGCCCTCCTCACAGATTTGTACATG
TTTTCCAACATGTATCCCGGCGGCGTTGCTCAGTACATCCGCAACGGGCACGAGCTGCTGGCGCGTGAGAGCGAA
GAGGTGGACTTTGCAGCGCTGGAGATGCCCCCTCTCATCTTCGAGGCGCCGTCGCTGCACCGGCGCACGGCTGAG
AGGACGGCGCTGGAGAACGCCGGAACCGCGATGCTGTGCAAGACGGTGTTCGTGCTGGTTGCTGGCGGTCTGGGC
GAACGTCTGGGCTACTCGAGCATCAAGGTGAGCCTGCCGGTGGAGACGGCGACGAACACAACGTATCTCGCCTAC
TACCTCCGGTGGGCCCAGCGGGTGGGGGGGAAGGAGGTACCATTTGTGATAATGACCTCTGACGACACGCACGAC
CGCACGCTGCAGCTCCTGCGCGAGCTGCAGTTGGAGGTGCCCAACTTGCATGTGCTCAAGCAGGGGCAGGTCTTC
TGTTTTGCCGACAGCGCCGCGCACCTCGCCCTGGACGAGACAGGGAAGCTGCTGCGCAAGCCACACGGTCACGGC
GACGTGCACTCCCTCATCTACAACGCGACTGTGAAGAGAGACGTGGTGCCGGACTCCGGCGACGGTACCGCGACG
GCGCAGCCACTCGTGAACGACTGGCTGGCGGCCGGCTACGAGTCCATTGTCTTCATCCAGGACACCAACGCCGGC
GCGACGATCACAATCCCCATCAGCCTCGCCTTGAGTGCCGAGCACTCGCTCGACATGAACTTCACCTGCATCCCT
CGTGTGCCGAAGGAGCCGATCGGGCTGCTATGCCGAACCAAGAAGAATAGCGGCGACCCGTGGCTGGTCGCGAAC
GTGGAGTACAACGTCTTTGCCGAGGTCTCGCGCGCGCTTAACAAGGATGGTGGCGATGAAGTCAGTGACCCCACT
GGCTTCTCCCCGTTCCCTGGCAGCGTCAACACCCTCGTGTTCAAGCTCTCCAGCTACGTGGACCGGCTGCGGGAG
TCGCACGGTATCGTGCCGGAGTTCATCAATCCCAAGTACTCGGACGAGACGCGCCGCTCCTTCAAGAAGCCCGCA
CGCATCGAGTCCCTGATGCAGGACATCGCGCTGCTCTTCTCCGAGGATGACTACCGTGTCGGCGGTACCGTCTTT
GAGCGATTCTCGTACCAGCCAGTGAAGAACTCGCTAGAGGAGGCGGCAGGGCTTGTGGCGCAGGGCAACGGCGCC
TACTGCGCCGCCACGGGAGAGGCTGCCTTCTACGAGCTGCAGCGGCGCCGTCTCAAGGCCATCGGGCTGCCGCTC
TTCTACAGCTCGCAGCCGGAGGTGACGGTGGCGAAGGACGCCTTTGGCGTGCGTCTCTTCCCGATAATCGTGCTG
GATACGATGTGCGCGTCAAGCGGATCCCTCGACGACCTTGCGCGCGTCTTTCCGACGCCGGAAAAGGTGCACATC
GATCAGCACAGCACCTTGATTGTTGAGGGCCGTGTCATCATCGAGAGCCTGGAGCTATACGGTGCACTCACGATT
CGCGGCCCGACAGACTCGATGGCGCTGCCGCACGTAGTACGAAACGCTGTGGTGCGCAATGCCGGCTGGTCGGTA
CACGCGATCTTGTCTCTCTGCGCTGGGCGCGATAGCAGGCTGTCCGAGGTGGACCGCATCCGCGGGTTTGTGCTG
AAGAAGACAGCCATGGCGGTGATGGACTGCAATACGAAGGGCGAGTCCGAGGCCGGTGCACCGTCTGGTGCGGCT
GACCCGGCAAAGTTGTAG

[UDP-Sugar Phosphorylase Cloned from Leishmania major, Amino Acid Sequence]

>Leishmania_USP.pro
SEQ ID NO: 10
MTNPSNSNLQALREELCTPGLDQGHLFEGWPETVDECNERQIALLTDLYMFSNMYPGGVAQYIRNGHELLARESE
EVDFAALEMPPLIFEAPSLHRRTAERTALENAGTAMLCKTVFVLVAGGLGERLGYSSIKVSLPVETATNTTYLAY
YLRWAQRVGGKEVPFVIMTSDDTHDRTLQLLRELQLEVPNLHVLKQGQVFCFADSAAHLALDETGKLLRKPHGHG
DVHSLIYNATVKRDVVPDSGDGTATAQPLVNDWLAAGYESIVFIQDTNAGATITIPISLALSAEHSLDMNFTCIP
RVPKEPIGLLCRTKKNSGDPWLVANVEYNVFAEVSRALNKDGGDEVSDPTGFSPFPGSVNTLVFKLSSYVDRLRE
SHGIVPEFINPKYSDETRRSFKKPARIESLMQDIALLFSEDDYRVGGTVFERFSYQPVKNSLEEAAGLVAQGNGA
YCAATGEAAFYELQRRRLKAIGLPLFYSSQPEVTVAKDAFGVRLFPIIVLDTMCASSGSLDDLARVFPTPEKVHI
DQHSTLIVEGRVIIESLELYGALTIRGPTDSMALPHVVRNAVVRNAGWSVHAILSLCAGRDSRLSEVDRIRGFVL
KKTAMAVMDCNTKGESTEAGAPSGAADPAKL

[UDP-GlcNAc-Epimerase (NmA) Clones from Neisseria Meningitidis Serotype A, Coding Sequence]

>UDP-GlcNAc-Epimerase-NmA (AF019760 REGION: 479 . . . 1597)
SEQ ID NO: 11
Atgaaagtcttaaccgtctttggcactcgccctgaagctattaaaatggcgcctgtaattctagagttacaaaaa
cataacacaattacttcaaaagtttgcattactgcacagcatcgtgaaatgctagatcaggttttgagcctattc
gaaatcaaagctgattatgatttaaatatcatgaaacccaaccagagcctacaagaaatcacaacaaatatcatc
tcaagccttaccgatgttcttgaagatttcaaacctgactgcgtccttgctcacggagacaccacaacaactttt
gcagctagccttgctgcattctatcaaaaaatacctgttggccacattgaagcaggcctgagaacttataattta
tactctccttggccagaggaagcaaataggcgtttaacaagcgttctaagccagtggcattttgcacctactgaa
gattctaaaaataacttactatctgaatcaataccttctgacaaagttattgttactggaaatactgtcatagat
gcactaatggtatctctagaaaaactaaaaataactacaattaaaaaacaaatggaacaagcttttccatttatt
caggacaactctaaagtaattttaattaccgctcatagaagagaaaatcatggggaaggtattaaaaatattgga
ctttctatcttagaattagctaaaaaatacccaacattctcttttgtgattccgctccatttaaatcctaacgtt
agaaaaccaattcaagatttattatcctctgtgcacaatgttcatcttattgagccacaagaatacttaccattc
gtatatttaatgtctaaaagccatataatattaagtgattcaggcggcatacaagaagaagctccatccctagga
aaaccagttcttgtattaagagatactacagaacgtcctgaagctgtagctgcaggaactgtaaaattagtaggt
tctgaaactcaaaatattattgagagctttacacaactaattgaataccctgaatattatgaaaaaatggctaat
attgaaaacccttacgggataggtaatgcctcaaaaatcattgtagaaactttattaaagaatagataa

[UDP-GlcNAc-Epimerase (NmA) Cloned from Neisseria meningitidis Serogroup A Amino Acid Sequence]

>UDP-GlcNAc-Epimerase-NmA (AAC38285).pro
SEQ ID NO: 12
mkvltvfgtrpeaikmapvilelqkhntitskvcitaqhremldqvlslfeikadydlnimkpnqslqeittnii
ssltdvledfkpdcvlahgdttttfaaslaafyqkipvghieaglrtynlyspwpeeanrrltsvlsqwhfapte
dsknnllsesipsdkvivtgntvidalmvsleklkittikkqmeqafpfiqdnskvilitahrrenhgegiknig
lsilelakkyptfsfviplhlnpnvrkpiqdllssvhnvhliepqeylpfvylmskshiilsdsggiqeeapslg
kpvlvlrdtterpeavaagtvklygsetqniiesftqlieypeyyekmanienpygignaskiivetllknr

[Capsule Polymerase Cloned from Neisseria meningitidis Serogroup W-135 Carrying an N-Terminal StrepII and a C-Terminal 6×His-tag, Coding Sequence]

>Strep_CP_NmW135_His_cds(Y13970).seq
SEQ ID NO: 13
ATGGCTAGCTGGAGCCACCCGCAGTTCGAAAAAGGCGCCCTGGTTCCGCGTGGATCCgctgttattatatttgtt
aacggaattcgggctgtaaatggccttgttaaatcatctatcaatactgcaaacgcttttgctgaagaaggactg
gatgttcatttaattaattttgttggcaatattactggagcagagcatttataccccccattccacttacatccc
aatgtcaaaacctccagcatcatagatttatttaatgacattccagaaaatgttagctgccgaaatactcctttt
tattctattcatcaacaattcttcaaagctgaatatagtgcccactataagcatgttttgatgaaaattgaatct
ttattatctgcagaagatagcattatcttcactcatcctcttcaactggaaatgtatcgtttagcgaataatgat
atcaagtcaaaagccaaactaattgtacaaattcatggtaattatatggaagaaatccataactatgaaattttg
gcacgaaatatcgattatgttgactatcttcaaacggtatctgatgaaatgctggaagaaatgcattcccatttc
aaaatcaaaaaagacaaattagtttttattccaaacatcacttatcccatttcattagaaaaaaaagaagctgat
ttctttattaaggataatgaagacatcgataatgctcagaaatttaaacgtatctatattgttggcagcattcag
ccaagaaaaaaccaattggatgccattaaaatcatcaataaaattaaaaatgaaaattacattttacagatatat
ggcaaatctattaataaagattactttgaattaattaaaaaatatattaaagacaataagttacaaaaccgtatc
ttattcaaaggtgaatcttccgagcaggaaatttatgaaaatacagatatcctgatcatgacatcagaaagtgag
ggatttccatatatatttatggaaggcatggtgtatgatattccaatcgttgtatatgattttaaatatggagcg
aatgattacagtaactataatgaaaatggttgtgtttttaaaactggtgatatttctggaatggcaaaaaaaata
attgagctattaaataacccagaaaaatataaagaattagttcaatataatcacaatcgcttcttaaaagaatat
gcaaaagatgtggttatggctaaatatttcactattcttccgcgcagctttaataacgtatcattatcgtctgct
ttcagccgaaaagaattggacgaattccaaaatattactttttctattgaagattctaatgatttagctcatatt
tggaatttcgagctaaccaatcctgcacaaaatatgaatttttttgctttagttggcaagcgaaaatttccaatg
gatgctcatatccaaggaacacagtgtacgattaagatagctcataaaaagacagggaatttattgtcgctttta
ctaaaaaaacgaaatcagttgaatttatcaaggggatataccttaattgcagaagataatagctatgaaaaatat
attggagcaatatctaataaaggtaactttgaaattattgcaaataaaaagagctcattagttactataaacaaa
agtaccttagagttgcatgagattccccatgaactacatcagaataaattactgattgctttacccaacatgcaa
acgcctctaaaaattactgatgataatttaatacctatccaagcctccataaaattagaaaagattggaaatact
tattacccatgtttcttgccatctggcatatttaataatatctgcttagattacggtgaagaatccaaaattatt
aattttagtaaatattcttataaatatatctatgactcaattcgtcatattgagcaacatacagatatatcggat
attatcgtttgcaatgtttattcttgggaacttattcgtgcctcagttattgagagccttatggaatttaccgga
aaatgggaaaaacactttcagacttctcctaaaattgattatcgatttgatcatgaaggtaagcgttcgatggat
gatgtcttttcagaagaaacatttattatggaatttccgcgtaaaaatggtatagataagaaaacagcagccttc
caaaatataccaaacagtattgtaatggagtatccgcagaccaatggttacagtatgcgcagtcattcactgaaa
agtaatgtagttgcggcaaaacattttcttgaaaaattaaataaaattaaggtagatattaaatttaaaaagcat
gaccttgcaaacatcaaaaaaatgaatcgaattatttatgagcatttaggcattaacataaatatcgaagcattt
ctaaaaccacgattagaaaaatttaagcgtgaagaaaaatattttcatgatttcttcaaaagaaataattttaaa
gaggtaatttttccaagcacttattggaatccaggtattatttgtgctgcacataaacaaggtattaaggtatct
gatattcaatatgctgccattactccttatcatcctgcgtattttaaatcaccaaaatcacattacgttgctgat
aaattgttattatggtctgaatattggaatcatgagcttttaccaaatccaacacgagagattggttctggtgcc
gcatattggtatgcattagatgatgtgagattttcagaaaaactgaattatgactatatctttctatctcaaagt
aggatttcttcgcgcttgcttagttttgcaattgagtttgcattaaaaaatcctcaactacagcttttattttct
aagcatccagatgaaaatatagatttaaagaacagaattattcctgataatcttataatctccacggaatcttct
atacaaggcatcaatgaatctcgcgttgctgtaggtgtttattcaactagcttatttgaggcattagcatgcggc
aaacaaacttttgttgttaaatatccgggatatgaaattatgtcaaatgaaatagattcagggttattctttgca
gtagaaacacctgaagaaatgcttgagaaaacaagcccgaattgggtggctgtggcagatattgaaaaccagttt
tttggccaagaaaaaCTCGAGCACCACCACCACCACCACTGA

[Capsule Polymerase Cloned from Neisseria meningitidis Serogroup W-135 Carrying an N-Terminal StrepII and a C-Terminal 6×His-tag, Amino Acid Sequence]

>Strep_CP_NmW135_His(Y13970).pro
SEQ ID NO: 14
MASWSHPQFEKGALVPRGSAVIIFVNGIRAVNGLVKSSINTANAFAEEGLDVHLINFVGNITGAEHLYPPFHLHP
NVKTSSIIDLFNDIPENVSCRNTPFYSIHQQFFKAEYSAHYKHVLMKIESLLSAEDSIIFTHPLQLEMYRLANND
IKSKAKLIVQIHGNYMEEIHNYEILARNIDYVDYLQTVSDEMLEEMHSHFKIKKDKLVFIPNITYPISLEKKEAD
FFIKDNEDIDNAQKFKRISIVGSIQPRKNQLDAIKIINKIKNENYILQIYGKSINKDYFELIKKYIKDNKLQNRI
LFKGESSEQEIYENTDILIMTSESEGFPYIFMEGMVYDIPIVVYDFKYGANDISNYNENGCVFKTGDISGMAKKI
IELLNNPEKYKELVQYNHNRFLKEYAKDVVMAKYFTILPRSFNNVSLSSAFSRKELDEFQNITFSIEDSNDLAHI
WNFELTNPAQNMNFFALVGKRKFPMDAHIQGTQCTIKIAHKKTGNLLSLLLKKRNQLNLSRGYTLIAEDNSYEKY
IGAISNKGNFEIIANKKSSLVTINKSTLELHEIPHELHQNKLLIALPNMQTPLKITDDNLIPIQASIKLEKIGNT
YYPCFLPSGIFNNICLDYGEESKIINFSKYSYKYIYDSIRHIEQHTDISDIIVCNVYSWELIRASVIESLMEFTG
KWEKHFQTSPKIDYRFDHEGKRSMDDVFSEETFIMEFPRKNGIDKKTAAFQNIPNSIVMEYPQTNGYSMRSHSLK
SNVVAAKHFLEKLNKIKVDIKFKKHDLANIKKMNRIIYEHLGININIEAFLKPRLEKFKREEKYFHDFFKRNNFK
EVIFPSTYWNPGIICAAHKQGIKVSDIQYAAITPYHPAYFKSPKSHYVADKLFLWSEYWNHELLPNPTREIGSGA
AYWYALDDVRFSEKLNYDYIFLSQSRISSRLLSFAIEFALKNPQLQLLFSKHPDENIDLKNRIIPDNLIISTESS
IQGINESRVAVGVYSTSLFEALACGKQTFVVKYPGYEIMSNEIDSGLFFAVETPEEMLEKTSPNWVAVADIENQF
FGQEKLEHHHHHH

[Capsule Polymerase Cloned from Neisseria meningitidis Serogroup Y Carrying an N-Terminal StrepII and a C-Terminal 6×His-Tag, Coding Sequence]

>Strep_CP_NmY_His_cds(Y13969).seq
SEQ ID NO: 15
ATGGCTAGCTGGAGCCACCCGCAGTTCGAAAAAGGCGCCCTGGTTCCGCGTGGATCCgctgttattatatttgtt
aacggaattcgggctgtaaatggccttgttaaatcatctatcaatactgcaaacgcttttgctgaagaaggactg
gatgttcatttaattaattttgttggcaatattactggagcagagcatttatcccccccattccacttacatccc
aatgtcaaaacctccagcatcatagatttatttaatgacattccagaaaatgttagctgccgaaatattcctttt
tattctatccatcaacaattcttcaaagccgaatacagtgcccactataagcatgttttgatgaaaattgaatct
ttattatctgaagaagatagcattatcttcactcatcctcttcaactggaaatgtatcgtttagcgaataataat
attaagtcaaaagccaagctaattgtacaaattcatggtaactatatggaagaaatccataactatgaaatttgg
gcacgaaatatcgattatgttgattatcttcaaacggtatctgatgaaatgctggaagaaatgcattcccatttc
aaaatcaaaaaagacaaattagtttttattccaaacatcacttatcccatttcattagaaaaaaaagaagctgat
ttctttattaaggataatgaagacattgataatgctcagaaatttaaacgtatctctattgttggcagtattcag
ccaagaaaaaaccaattggatgccattaaaatcatcaataaaattaaaaatgaaaattacattttacagatatat
ggcaaatctattaataaagattactttgaattaattaaaaaatatattaaagacaataagttacaaaaccgtatc
ttattcaaaggtgaatcttccgagcaggaaatttatgagaatacagatatcctaatcatgacatctcaaagcgaa
ggctttggttatatatttctagagggtatggtgtacgatatccctatccttgcctataattttaaatatggagcg
aatgattttagcaattataatgaaaacgcttcagtttttaaaactggtgatatttctggaatggcaaaaaaaata
attgagctattaaataacccagaaaaatataaagaattagttcaatataatcacaatcgcttcttaaaagaatat
gcaaaagatgtggttatggctaaatatttcactattcttccgcgcagctttaataacgtatcattatcgtctgct
ttcagccgaaaagaattggacgaattccaaaatattactttttctattgaagattctaatgatttagctcatatt
tggaatttcgagctaaccaatcctgcacaaaatatgaatttttttgctttagttggcaagcgaaaatttccaatg
gatgctcatatccaaggaacacagtgtacgattaagatagctcataaaaagacagggaatttattgtcgctttta
ctaaaaaaacgaaatcagttgaatttatcaaggggatataccttaattgcagaagataatagctatgaaaaatat
attggagcaatatctaataaaggtaactttgaaattattgcaaataaaaagaactcattagttactataaacaaa
agtaccttagagttgcatgagattccccatgaactacatcagaataaattactgattgctttacccaacatgcaa
acgcctctaaaaattactgatgataatttaatacctatccaagcctccataaaattagaaaagattggaaatact
tattacccatgtttcttgccatctggcatatttaataatatctgcttagattacggtgaagaatccaaaattatt
aattttagtaaatattcttataaatatatctatgactcaattcgtcatattgagcaacatacagatatatcggat
attatcgtttgcaatgtttattcttgggaacttattcgtgcctcagttattgagagccttatggaatttaccgga
aaatgggaaaaacactttcagacttctcctaaaattgattatcgatttgatcatgaaggtaagcgttcgatggat
gatgtcttttcagaagaaacatttattatggaatttccgcgtaaaaatggtatagataagaaaacagcagccttc
caaaatataccaaacagtattgtaatggagtatccgcagaccaatggttacagtatgcgcagtcattcactgaaa
agtaatgtagttgcggcaaaacattttcttgaaaaattaaataaaattaaggtagatattaaatttaaaaagcat
gaccttgcaaacatcaaaaaaatgaatcgaattatttatgagcatttaggcattaacataaatatcgaagcattt
ctaaaaccacgattagaaaaatttaagcgtgaagaaaaatattttcatgatttcttcaaaagaaataattttaaa
gaggtaatttttccaagcacttattggaatccaggtattatttgtgctgcacataaacaaggtattaaggtatct
gatattcaatatgctgccattactccttatcatcctgcgtattttaaatcaccaaaatcacattacgttgctgat
aaattgttcttatggtctgaatattggaatcatgagcttttaccaaatccaacacgagagattaattctggtgcc
gcatattggtatgcattagatgatgtgagattttcagaaaaactgaattatgactatatctttctatctcaaagt
aggatttcttcgcgcttgcttagttttgcaattgagtttgcattaaaaaatcctcaactacagcttttattttct
aagcatctagatgaaaatatagatttaaagaacagaattattcctgataatcttataatctccacggaatcttct
atacaaggcatcaatgaatctcgcgttgctgtaggtgtttattcaactagcttatttgaggcattagcatgcggc
aaacaaacttttgttgttaaatatccgggatatgaaattatgtcaaatgaaatagattcagggttattctttgca
gtagaaacacctgaagaaatgcttgagaaaacaagcccgaattgggtggctgtggcagatattgaaaaccagttt
tttggccaagaaaaaCTCGAGCACCACCACCACCACCACTGA

[Capsule Polymerase Cloned from Neisseria meningitidis Serogroup Y Carrying an N-Terminal StrepII and a C-Terminal 6×His-Tag, Amino Acid Sequence]

>Strep_CP_NmY_His(Y13969).pro
SEQ ID NO: 16
MASWSHPQFEKGALVPRGSAVIIFVNGIRAVNGLVKSSINTANAFAEFGLDVHLINFVGNITGAEHLSPPFHLHP
NVKTSSIIDLFNDIPENVSCRNIPFYSIHQQFFKAEYSAHYKHVLMKIESLLSEEDSIIFTHPLQLEMYRLANNN
IKSKAKLIVQIHGNYMEEIHNYEIWARNIDYVDYLQTVSDEMLEEMHSHFKIKKDKLVFIPNITYPISLEKKEAD
FFIKDNEDIDNAQKFKRISIVGSIQPRKNQLDAIKIINKIKNENYILQIYGKSINKDYFELIKKYIKDNKLQNRI
LFKGESSEQEIYENTDILIMTSQSEGEGYIFLEGMVYDIPILAYNFKYGANDFSNYNENASVFKTGDISGMAKKI
IELLNNPEKYKELVQYNHNRFLKEYAKDVVMAKYFTILPRSFNNVSLSSAFSRKELDEFQNITFSIEDSNDLAHI
WNFELTNPAQNMNFFALVGKRKFPMDAHIQGTQCTIKIAHKKTGNLLSLLLKKRNQLNLSRGYTLIAEDNSYEKY
IGAISNKGNFEIIANKKNSLVTINKSTLELHEIPHELHQNKLLIALPNMQTPLKITDDNLIPIQASIKLEKIGNT
YYPCFLPSGIFNNICLDYGEESKIINFSKYSYKYIYDSIRHIEQHTDISDIIVCNVYSWELIRASVIESLMEFTG
KWEKHFQTSPKIDYRFDHEGKRSMDDVFSEETFIMEFPRKNGIDKKTAAFQNIPNSIVMEYPQTNGYSMRSHSLK
SNVVAAKHFLEKLNKIKVDIKFKKHDLANIKKMNRIIYEHLGININIEAFLKPRLEKFKREEKYFHDFFKRNNFK
EVIFPSTYWNPGIICAAHKQGIKVSDIQYAAITPYHPAYFKSPKSHYVADKLFLWSEYWNHELLPNPTREIGSGA
AYWYALDDVRFSEKLNYDYIFLSQSRISSRLLSFAIEFALKNPQLQLLFSKHLDENIDLKNRIIPDNLIISTESS
IQGINESRVAVGVYSTSLFEALACGKQTFVVKYPGYEIMSNEIDSGLFFAVETPEEMLEKTSPNWVAVADIENQF
FGQEKLEHHHHHH

[Capsule Polymerase Cloned from Neisseria meningitidis Serogroup W-135 Carrying a C-Terminal 6×His-Tag, Coding Sequence]

>CP_NmW135_His_cds(Y13970).seq
SEQ ID NO: 17
atggctgttattatatttgttaacggaattcgggctgtaaatggccttgttaaatcatctatcaatactgcaaac
gcttttgctgaagaaggactggatgttcatttaattaattttgttggcaatattactggagcagagcatttatac
cccccattccacttacatcccaatgtcaaaacctccagcatcatagatttatttaatgacattccagaaaatgtt
agctgccgaaatactcctttttattctattcatcaacaattcttcaaagctgaatatagtgcccactataagcat
gttttgatgaaaattgaatctttattatctgcagaagatagcattatcttcactcatcctcttcaactggaaatg
tatcgtttagcgaataatgatatcaagtcaaaagccaaactaattgtacaaattcatggtaattatatggaagaa
atccataactatgaaattttggcacgaaatatcgattatgttgactatcttcaaacggtatctgatgaaatgctg
gaagaaatgcattcccatttcaaaatcaaaaaagacaaattagtttttattccaaacatcacttatcccatttca
ttagaaaaaaaagaagctgatttctttattaaggataatgaagacatcgataatgctcagaaatttaaacgtatc
tctattgttggcagcattcagccaagaaaaaaccaattggatgccattaaaatcatcaataaaattaaaaatgaa
aattacattttacagatatatggcaaatctattaataaagattactttgaattaattaaaaaatatattaaagac
aataagttacaaaaccgtatcttattcaaaggtgaatcttccgagcaggaaatttatgaaaatacagatatcctg
atcatgacatcagaaagtgagggatttccatatatatttatggaaggcatggtgtatgatattccaatcgttgta
tatgattttaaatatggagcgaatgattacagtaactataatgaaaatggttgtgtttttaaaactggtgatatt
tctggaatggcaaaaaaaataattgagctattaaataacccagaaaaatataaagaattagttcaatataatcac
aatcgcttcttaaaagaatatgcaaaagatgtggttatggctaaatatttcactattcttccgcgcagctttaat
aacgtatcattatcgtctgctttcagccgaaaagaattggacgaattccaaaatattactttttctattgaagat
tctaatgatttagctcatatttggaatttcgagctaaccaatcctgcacaaaatatgaatttttttgctttagtt
ggcaagcgaaaatttccaatggatgctcatatccaaggaacacagtgtacgattaagatagctcataaaaagaca
gggaatttattgtcgcttttactaaaaaaacgaaatcagttgaatttatcaaggggatataccttaattgcagaa
gataatagctatgaaaaatatattggagcaatatctaataaaggtaactttgaaattattgcaaataaaaagagc
tcattagttactataaacaaaagtaccttagagttgcatgagattccccatgaactacatcagaataaattactg
attgctttacccaacatgcaaacgcctctaaaaattactgatgataatttaatacctatccaagcctccataaaa
ttagaaaagattggaaatacttattacccatgtttcttgccatctggcatatttaataatatctgcttagattac
ggtgaagaatccaaaattattaattttagtaaatattcttataaatatatctatgactcaattcgtcatattgag
caacatacagatatatcggatattatcgtttgcaatgtttattcttgggaacttattcgtgcctcagttattgag
agccttatggaatttaccggaaaatgggaaaaacactttcagacttctcctaaaattgattatcgatttgatcat
gaaggtaagcgttcgatggatgatgtcttttcagaagaaacatttattatggaatttccgcgtaaaaatggtata
gataagaaaacagcagccttccaaaatataccaaacagtattgtaatggagtatccgcagaccaatggttacagt
atgcgcagtcattcactgaaaagtaatgtagttgcggcaaaacattttcttgaaaaattaaataaaattaaggta
gatattaaatttaaaaagcatgaccttgcaaacatcaaaaaaatgaatcgaattatttatgagcatttaggcatt
aacataaatatcgaagcatttctaaaaccacgattagaaaaatttaagcgtgaagaaaaatattttcatgatttc
ttcaaaagaaataattttaaagaggtaatttttccaagcacttattggaatccaggtattatttgtgctgcacat
aaacaaggtattaaggtatctgatattcaatatgctgccattactccttatcatcctgcgtattttaaatcacca
aaatcacattacgttgctgataaattgttcttatggtctgaatattggaatcatgagcttttaccaaatccaaca
cgagagattggttctggtgccgcatattggtatgcattagatgatgtgagattttcagaaaaactgaattatgac
tatatctttctatctcaaagtaggatttcttcgcgcttgcttagttttgcaattgagtttgcattaaaaaatcct
caactacagcttttattttctaagcatccagatgaaaatatagatttaaagaacagaattattcctgataatctt
ataatctccacggaatcttctatacaaggcatcaatgaatctcgcgttgctgtaggtgtttattcaactagctta
tttgaggcattagcatgcggcaaacaaacttttgttgttaaatatccgggatatgaaattatgtcaaatgaaata
gattcagggttattctttgcagtagaaacacctgaagaaatgcttgagaaaacaagcccgaattgggtggctgtg
gcagatattgaaaaccagttttttggccaagaaaaaCTCGAGCACCACCACCACCACCACTGA

[Capsule Polymerase Cloned from Neisseria meningitidis Serogroup W-135 Carrying a C-Terminal 6×His-Tag, Amino Acid Sequence]

>CP_NmW135_His(Y13970).pro
SEQ ID NO: 18
MAVIIFVNGIRAVNGLVKSSINTANAFAEEGLDVHLINFVGNITGAEHLYPPFHLHPNVKTSSIIDLFNDIPENV
SCRNTPFYSIHQQFFKAEYSAHYKHVLMKIESLLSAEDSIIFTHPLQLEMYRLANNDIKSKAKLIVQIHGNYMEE
IHNYEILARNIDYVDYLQTVSDEMLEEMHSHFKIKKDKLVFIPNITYPISLEKKEADFFIKDNEDIDNAQKFKRI
SIVGSIQPRKNQLDAIKIINKIKNENYILQIYGKSINKDYFELIKKYIKDNKLQNRILFKGESSEQEIYENTDIL
IMTSESEGFPYIEMEGMVYDIPIVVYDFKYGANDYSNYNENGCVFKTGDISGMAKKIIELLNNPEKYKELVQYNH
NRFLKEYAKDVVMAKYFTILPRSFNNVSLSSAFSRKELDEFQNITFSIEDSNDLAHIWNFELTNPAQNMNFFALV
GKRKFPMDAHIQGTQCTIKIAHKKIGNLLSLLLKKRNQLNLSRGYTLIAEDNSYEKYIGAISNKGNFEIIANKKS
SLVTINKSTLELHEIPHELHQNKLLIALPNMQTPLKITDDNLIPIQASIKLEKIGNTYYPCFLPSGIFNNICLDY
GEESKIINFSKYSYKYIYDSIRHIEQHTDISDIIVCNVYSWELIRASVIESLMEFTGKWEKHFQTSPKIDYRFDH
EGKRSMDDVFSEETFIMEFPRKNGIDKKTAAFQNIPNSIVMEYPQTNGYSMRSHSLKSNVVAAKHFLEKLNKIKV
DIKFKKHDLANIKKMNRIIYEHLGININIEAFLKPRLEKFKREEKYFHDFFKRNNFKEVIFPSTYWNPGIICAAH
KQGIKVSDIQYAAITPYHPAYEKSPKSHYVADKLFLWSEYWNHELLPNPTREIGSGAAYWYALDDVRFSEKLNYD
YIFLSQSRISSRLLSFAIEFALKNPQLQLLFSKHPDENIDLKNRIIPDNLIISTESSIQGINESRVAVGVYSTSL
FEALACGKQTFVVKYPGYEIMSNEIDSGLFFAVETPEEMLEKTSPNWVAVADIENQFFGQEKLEHHHHHH

[Capsule Polymerase Cloned from Neisseria meningitidis Serogroup X Carrying an N-Terminal MBP and a C-Terminal 6×His-Tag, Coding Sequence]

>MBP_CP_NmX_His_cds(AAP44500).seq
SEQ ID NO: 19
ATGAAAACTGAAGAAGGTAAACTGGTAATCTGGATTAACGGCGATAAAGGCTATAACGGTCTCGCTGAAGTCGGT
AAGAAATTCGAGAAAGATACCGGAATTAAAGTCACCGTTGAGCATCCGGATAAACTGGAAGAGAAATTCCCACAG
GTTGCGGCAACTGGCGATGGCCCTGACATTATCTTCTGGGCACACGACCGCTTTGGTGGCTACGCTCAATCTGGC
CTGTTGGCTGAAATCACCCCGGACAAAGCGTTCCAGGACAAGCTGTATCCGTTTACCTGGGATGCCGTACGTTAC
AACGGCAAGCTGATTGCTTACCCGATCGCTGTTGAAGCGTTATCGCTGATTTATAACAAAGATCTGCTGCCGAAC
CCGCCAAAAACCTGGGAAGAGATCCCGGCGCTGGATAAAGAACTGAAAGCGAAAGGTAAGAGCGCGCTGATGTTC
AACCTGCAAGAACCGTACTTCACCTGGCCGCTGATTGCTGCTGACGGGGGTTATGCGTTCAAGTATGAAAACGGC
AAGTACGACATTAAAGACGTGGGCGTGGATAACGCTGGCGCGAAAGCGGGTCTGACCTTCCTGGTTGACCTGATT
AAAAACAAACACATGAATGCAGACACCGATTACTCCATCGCAGAAGCTGCCTTTAATAAAGGCGAAACAGCGATG
ACCATCAACGGCCCGTGGGCATGGTCCAACATCGACACCAGCAAAGTGAATTATGGTGTAACGGTACTGCCGACC
TTCAAGGGTCAACCATCCAAACCGTTCGTTGGCGTGCTGAGCGCAGGTATTAACGCCGCCAGTCCGAACAAAGAC
CTGGCGAAAGAGTTCCTCGAAAACTATCTGCTGACTGATGAAGGTCTGGAAGCGGTTAATAAAGACAAACCGCTG
GGTGCCGTAGCGCTGAAGTCTTACGAGGAAGAGTTGGCGAAAGATCCACGTATTGCCGCCACCATGGAAAACGCC
CAGAAAGGTGAAATCATGCCGAACATCCCGCAGATGTCCGCTTTCTGGTATGCCGTGCGTACTGCGGTGATCAAC
GCCGCCAGCGGTCGTCAGACTGTCGATGAAGCCCTGAAAGACGCGCAGACTAATTCGAGCTCGGTACCCGGCCGG
GGATCCattatgagcaaaattagcaaattggtaacccacccaaaccttttctttcgagattatttcttaaaaaaa
gcaccgttaaattatggcgaaaatattaaacctttaccagtcgaaacctcttctcatagcaaaaaaaatacagcc
cataaaacacccgtatcatccgaccaaccaattgaagatccatacccagtaacatttccaattgatgtagtttat
acttgggtagattcagatgatgaaaaattcaatgaagaacgcctaaagtttcaaaattcaagcacatctgagact
ctacaaggcaaagcagaaagcaccgatattgcaagattccaatcacgcgacgaattaaaatattcgattcgaagc
ctgatgaagtatgccccatgggtaaatcatatttacattgtaacaaatggtcaaataccaaaatggttagatacc
aacaatacaaaggtaacgattatccctcactcaactattatcgacagtcaatttctccctacttttaattctcac
gtcattgaatcctctctatataaaatcccaggattatcagagcattacatttatttcaatgatgatgtcatgcta
gctagagatttaagcccatcttatttctttacaagcagcggattagcaaaactgtttattaccaactctcgtcta
ccaaatggctataagaatgtgaaagacacaccaacccaatgggcctcaaaaaattcccgtgagattttacatgca
gaaacaggattttgggctgaagccatgtttgcacatacttttcatccacaacgtaaaagtgtacatgaatctatt
gaacacctatggcatgaacaattaaatgtttgtcgtcaaaaccgtttccgtgatatttcagatattaacatggcg
acattcctgcaccaccattttgccattttgacaggccaagctcttgctacacgcactaaatgtatttactttaac
gttcgctctcctcaagcagctcagcattacaaaacattattagctcgaaaaggaagcgaatacagcccacattct
atctgcttaaatgatcatacatcgagcaataaaaatattttatctaattacgaagccaaattacaaagcttttta
gaaacatactatccagatgtatcagaagcagaaattctccttcctactaaatctgaagtagctgaattagttaaa
cataaagattatttaactgtatatactaaattattacctattatcaataagcagctggtcaataaatataataaa
ccttattcatatcttttctattatttaggtttatctgcccggtttttatttgaagaaacgcaacaagaacactac
cgggaaactgctgaagaaaatttacaaatcttttgtggcctaaacccaaaacatacactagccctcaaatactta
gcggatgtcaccctcacatcacagcctagtggacaaCTCGAGCACCACCACCACCACCAC

[Capsule Polymerase Cloned from Neisseria meningitidis Serogroup X Carrying an N-Terminal MBP and a C-Terminal 6×His-Tag, Amino Acid Sequence]

>MBP_CP_NmX_His_(AAP44500).pro
SEQ ID NO: 20
MKTEEGKLVIWINGDKGYNGLAEVGKKFEKDTGIKVTVEHPDKLEEKFPQVAATGDGPDIIFWAHDRFGGYAQSG
LLAEITPDKAFQDKLYPFTWDAVRYNGKLIAYPIAVEALSLIYNKDLLPNPPKTWEEIPALDKELKAKGKSALMF
NLQEPYFTWPLIAADGGYAFKYENGKYDIKDVGVDNAGAKAGLTFLVDLIKNKHMNADTDYSIAEAAFNKGETAM
TINGPWAWSNIDTSKVNYGVTVLPTFKGQPSKPFVGVLSAGINAASPNKELAKEFLENYLLTDEGLEAVNKDKPL
GAVALKSYEEELAKDPRIAATMENAQKGEIMPNIPQMSAFWYAVRTAVINAASGRQTVDEALKDAQTNSSSVPGR
GSIMSKISKLVTHPNLFFRDYFLKKAPLNYGENIKPLPVETSSHSKKNTAHKTPVSSDQPIEDPYPVTFPIDVVY
TWVDSDDEKENEERLKFQNSSTSETLQGKAESTDIARFQSRDELKYSIRSLMKYAPWVNHIYIVTNGQIPKWLDT
NNTKVTIIPHSTIIDSQFLPTFNSHVIESSLYKIPGLSEHYIYFNDDVMLARDLSPSYFFTSSGLAKLFITNSRL
PNGYKNVKDTPTQWASKNSRELLHAETGFWAEAMFAHTFHPQRKSVHESIEHLWHEQLNVCRQNRFRDISDINMA
TFLHHHFAILTGQALATRTKCIYENVRSPQAAQHYKTLLARKGSEYSPHSICLNDHTSSNKNILSNYEAKLQSFL
ETYYPDVSEAEILLPTKSEVAELVKHKDYLTVYTKLLPIINKQLVNKYNKPYSYLFYYLGLSARFLFEETQQEHY
RETAEENLQIFCGLNPKHTLALKYLADVTLTSQPSGQLEHHHHHH

[Capsule Polymerase Cloned from Neisseria Meningitidis Serogroup a Carrying an N-Terminal Strep-tag and a C-Terminal 6×His-Tag, Coding Sequence]

>Strep_CP_NmA_His_cds(NC_003116 REGION: 183321 . . . 184958).seq
SEQ ID NO: 21
atggctagctggagccacccgcagttcgaaaaaggcgccctggttccgcgtggatcttttatacttaataacaga
aaatggcgtaaacttaaaagagaccctagcgctttctttcgagatagtaaatttaactttttaagatatttttct
gctaaaaaatttgcaaagaattttaaaaattcatcacatatccataaaactaatataagtaaagctcaatcaaat
atttcttcaaccttaaaacaaaatcggaaacaagatatgttaattcctattaatttttttaattttgaatatata
gttaaaaaacttaacaatcaaaacgcaataggtgtatatattcttccttctaatcttactcttaagcctgcatta
tgtattctagaatcacataaagaagactttttaaataaatttcttcttactatttcctctgaaaatttaaagctt
caatacaaatttaatggacaaataaaaaatcctaagtccgtaaatgaaatttggacagatttatttagcattgct
catgttgacatgaaactcagcacagatagaactttaagttcatctatatctcaattttggttcagattagagttc
tgtaaagaagataaggattttatcttatttcctacagctaacagatattctagaaaactttggaagcactctatt
aaaaataatcaattatttaaagaaggcatacgaaactattcagaaatatcttcattaccctatgaagaagatcat
aattttgatattgatttagtatttacttgggtcaactcagaagataagaattggcaagagttatataaaaaatat
aagcccgactttaatagcgatgcaaccagtacatcaagattccttagtagagatgaattaaaattcgcattacgc
tcttgggaaatgaatggatccttcattcgaaaaatttttattgtctctaattgtgctcccccagcatggctagat
ttaaataaccctaaaattcaatgggtatatcacgaagaaattatgccacaaagtgcccttcctacttttagctca
catgctattgaaaccagcttgcaccatataccaggaattagtaactattttatttacagcaatgacgacttccta
ttaactaaaccattgaataaagacaatttcttctattcgaatggtattgcaaagttaagattagaagcatgggga
aatgttaatggtgaatgtactgaaggagaacctgactacttaaatggtgctcgcaatgcgaacactctcttagaa
aaggaatttaaaaaatttactactaaactacatactcactcccctcaatccatgagaactgatattttatttgag
atggaaaaaaaatatccagaagagtttaatagaacactacataataaattccgatctttagatgatattgcagta
acgggctatctctatcatcattatgccctactctctggacgagcactacaaagttctgacaagacggaacttgta
cagcaaaatcatgatttcaaaaagaaactaaataatgtagtgaccttaactaaagaaaggaattttgacaaactt
cctttgagcgtatgtatcaacgatggtgctgatagtcacttgaatgaagaatggaatgttcaagttattaagttc
ttagaaactcttttcccattaccatcatcatttgagaaactcgagcaccaccaccacaaccactga

[Capsule Polymerase Cloned from Neisseria Meningitidis Serogroup A Carrying an N-Terminal Strep-Tag and a C-Terminal 6×His-Tag, Amino Acid Sequence]

>Strep_CP_NmA_His_(YP_002341743).pro
SEQ ID NO: 22
MASWSHPQFEKGALVPRGSFILNNRKWRKLKRDPSAFFRDSKFNFLRYFSAKKFAKNFKNSSHIHKTNISKAQSN
ISSTLKQNRKQDMLIPINFENFEYIVKKINNQNAIGVYILPSNLTLKPALCILESHKEDFLNKFLLTISSENLKL
QYKFNGQIKNPKSVNEIWTDLFSIAHVDMKLSTDRTLSSSISQFWFRLEFCKEDKDFILEPTANRYSRKLWKHSI
KNNQLFKEGIRNYSEISSLPYEEDHNFDIDLVFTWVNSEDKNWQELYKKYKPDFNSDATSTSRFLSRDELKFALR
SWEMNGSFIRKIFIVSNCAPPAWLDLNNPKIQWVYHEEIMPQSALPTFSSHAIETSLHHIPGISNYFIYSNDDFL
LTKPLNKDNFFYSNGIAKLRLEAWGNVNGECTEGEPDYLNGARNANTLLEKEFKKFTTKLHTHSPQSMRTDILFE
MEKKYPEFFNRTLHNKFRSLDDIAVTGYLYHHYALLSGRALQSSDKTELVQQNHDFKKKENNVVTLTKERNFDKL
PLSVCINDGADSHLNEEWNVQVIKFLETLFPLPSSFEKLEHHHHHH

[UDP-GlcNAc-Epimerase Cloned from Neisseria meningitidis Serogroup a Carrying an N-Terminal Strep-Tag and a C-Terminal 6×His-Tag, Coding Sequence]

>Strep UDP-GlCNAc-Epimerase-NmA His(AF019760 REGION: 479 . . . 1597).seq
SEQ ID NO: 23
atggctagctggagccacccgcagttcgaaaaaggcgccctggttccgcgtggatccaaagtcttaaccgtcttt
ggcactcgccctgaagctattaaaatggcgcctgtaattctagagttacaaaaacataacacaattacttcaaaa
gtttgcattactgcacagcatcgtgaaatgctagatcaggttttgagcctattcgaaatcaaagctgattatgat
ttaaatatcatgaaacccaaccagagcctacaagaaatcacaacaaatatcatctcaagccttaccgatgttctt
gaagatttcaaacctgactgcgtccttgctcacggagacaccacaacaacttttgcagctagccttgctgcattc
tatcaaaaaatacctgttggccacattgaagcaggcctgagaacttataatttatactctccttggccagaggaa
gcaaataggcgtttaacaagcgttctaagccagtggcattttgcacctactgaagattctaaaaataacttacta
tctgaatcaataccttctgacaaagttattgttactggaaatactgtcatagatgcactaatggtatctctagaa
aaactaaaaataactacaattaaaaaacaaatggaacaagcttttccatttattcaggacaactctaaagtaatt
ttaattaccgctcatagaagagaaaatcatggggaaggtattaaaaatattggactttctatcttagaattagct
aaaaaatacccaacattctcttttgtgattccgctccatttaaatcctaacgttagaaaaccaattcaagattta
ttatcctctgtgcacaatgttcatcttattgagccacaagaatacttaccattcgtatatttaatgtctaaaagc
catataatattaagtgattcaggcggcatacaagaagaagctccatccctaggaaaaccagttcttgtattaaga
gatactacagaacgtcctgaagctgtagctgcaggaactgtaaaattagtaggttctgaaactcaaaatattatt
gagagctttacacaactaattgaataccctgaatattatgaaaaaatggctaatattgaaaacccttacgggata
ggtaatgcctcaaaaatcattgtagaaactttattaaagaatagactcgagcaccaccaccaccaccactga

[UDP-GlcNAc-Epimerase Cloned from Neisseria meningitidis Serogroup A Carrying an N-Terminal Strep-Tag and a C-Terminal 6×His-Tag, Amino Acid Sequence]

>Strep_UDP-GlcNAc-Epimerase-NmA His(AAC38285).pro
SEQ ID NO: 24
MASWSHPQFEKGALVPRGSKVLTVFGTRPEAIKMAPVILELQKHNTITSKVCITAQHREMLDQVLSLFEIKADYD
LNIMKPNQSLQEITTNIISSLTDVLEDFKPDCVLAHGDTTTTFAASLAAFYQKIPVGHIEAGLRTYNLYSPWPEE
ANRRLTSVLSQWHFAPTEDSKNNLLSESIPSDKVIVTGNTVIDALMVSLEKLKITTIKKQMEQAFPFIQDNSKVI
LITAHRRENHGEGIKNIGLSILELAKKYPTFSFVIPLHLNPNVRKPIQDLLSSVHNVHLIEPQEYLPFVYLMSKS
HIILSDSGGIQEEAPSLGKPVLVLRDTTERPEAVAAGTVKLVGSETQNIIESFTQLIEYPEYYEKMANIENPYGI
GNASKIIVETLLKNRLEHHHHHH.

1. In vitro method for producing capsular polysaccharides (CPS) of Neisseria meningitidis, said method comprising the steps:
(a) contacting at least one donor carbohydrate with at least one capsule polymerase (CP);
(b) incubation of said carbohydrate with said capsular polymerases, wherein said carbohydrate is activated or wherein said carbohydrate is activated during this step; and
(c) isolating the resulting capsular polysaccharide,
wherein the obtained capsular polysaccharides are synthetic or artificial capsular polysaccharides of Neisseria meningitidis serogroup W-135, specific capsular polysaccharides or wherein the obtained capsular polysaccharides are artificial chimeric capsular polysaccharides comprising capsular polysaccharides or capsular polysaccharide subunits of Neisseria meningitidis serogroups Y/W-135, W-135/Y, B/W-135, C/W-135, B/Y/W-135, C/Y/W-135, B/W-135/Y, C/W-135/Y, X/A or A/X.

2. Method according to claim 1, wherein said chimeric capsular polysaccharide comprises capsular polysaccharides or capsular polysaccharides subunits of Neisseria meningitidis serogroups W-135 and Y.

3. Method according to claim 1, wherein in step (a) said at least one donor carbohydrate and said at least one capsule polymerase are further contacted with an acceptor carbohydrate.

4. (canceled)

5. Method according to claim 1, wherein said at least one donor carbohydrate is activated by linkage of an activating nucleotide.

6. Method according to claim 5, wherein said activating nucleotide is selected from the group consisting of: CMP, UDP, TDP and AMP.

7. Method according to claim 1, wherein said capsule polymerase is
(a) CP-W-135 as shown in SEQ ID NOs 1 or 2; or
(b) a functional derivative of CP-W-135 having at least 80% sequence identity to SEQ ID NOs 1 or 2, whereby the functional derivative of CP-W-135 is capable of synthesizing capsular polysaccharides of serogroup W-135 and serogroup Y.

8. Method according to claim 1, wherein at least one donor carbohydrate is
(a) CMP-Neu5Ac and at least one donor carbohydrate is UDP-Gal or UDP-Glc; or
(b) selected from the group consisting of: Gal-1-P and sialic acid.

9-16. (canceled)

17. Method according to claim 8, wherein said sialic acid is Neu5Ac.

18. Method according to claim 1, wherein at least one donor carbohydrate is GlcNAc-1-P or ManNAc-1-P

19. (canceled)

20. Method according to any one of claims 8, wherein at least one donor carbohydrate is contacted with an activating enzyme during step (a) of claim 1 and activated during step (b) of claim 1, wherein Gal-1-P is activated by the UDP-sugar pyrophosphorylase and Neu5Ac is activated by CMP-NeuNAc synthetase.

21. Nucleic acid molecule encoding a pyrophosphorylase or a fragment thereof, said nucleic acid molecule comprising a nucleic acid molecule selected from the group consisting of:
(a) nucleic acid molecule having the nucleotide sequence of SEQ ID NO: 9;
(b) nucleic acid molecule encoding a polypeptide comprising the amino acid sequence of SEQ ID NO: 10;
(c) nucleic acid molecule having the nucleotide sequence of SEQ ID NO: 9, wherein one or more nucleotides are added, deleted or substituted;
(d) nucleic acid molecule encoding a polypeptide comprising the amino acid sequence of SEQ ID NO: 10, wherein one or more amino acid residue is added, deleted or substituted;
(e) nucleic acid molecule which is at least 45% identical to the nucleotide sequence of SEQ ID NO: 9;
(f) nucleic acid molecule encoding a polypeptide comprising an amino acid sequence which is at least 45% identical to the amino acid sequence of SEQ ID NO: 10;
(g) nucleic acid molecule complementary to the nucleic acid molecule of any one of (a) to (f);
(h) nucleic acid molecule which hybridizes under stringent conditions to any of the nucleic acid molecules of (a) to (g);
(i) nucleic acid molecule which differs from the sequence of a nucleic acid molecule of any one of (a) to (h) due to the degeneracy of the genetic code; and
(j) a functional fragment of a nucleic acid molecule of (a) to (i).

22. Vector containing the nucleic acid molecule of claim 21.

23. Host cell containing the vector of claim 22.

24. Polypeptide encoded by the nucleic acid molecule of claim 21.

25. Method according to claim 8, wherein Gal-1-P or Glc-1-P is contacted with a nucleic acid molecule encoding a pyrophosphorylase or a polypeptide encoded by said nucleic acid molecule during step (a) of claim 1 and activated during step (b) of claim 1, wherein said nucleic acid molecule encoding a pyrophosphorylase or a fragment thereof comprises a nucleic acid molecule selected from the group consisting of:
(a) nucleic acid molecule having the nucleotide sequence of SEQ ID NO: 9;
(b) nucleic acid molecule encoding a polypeptide comprising the amino acid sequence of SEQ ID NO: 10;
(c) nucleic acid molecule having the nucleotide sequence of SEQ ID NO: 9, wherein one or more nucleotides are added, deleted or substituted;
(d) nucleic acid molecule encoding a polypeptide comprising the amino acid sequence of SEQ ID NO: 10, wherein one or more amino acid residue is added, deleted or substituted;
(e) nucleic acid molecule which is at least 45% identical to the nucleotide sequence of SEQ ID NO: 9;
(f) nucleic acid molecule encoding a polypeptide comprising an amino acid sequence which is at least 45% identical to the amino acid sequence of SEQ ID NO: 10;
(g) nucleic acid molecule complementary to the nucleic acid molecule of any one of (a) to (f);
(h) nucleic acid molecule which hybridizes under stringent conditions to any of the nucleic acid molecules of (a) to (g);
(i) nucleic acid molecule which differs from the sequence of a nucleic acid molecule of any one of (a) to (h) due to the degeneracy of the genetic code; and
(j) a functional fragment of a nucleic acid molecule of (a) to (i).

26. Method according to claim 25, wherein in step (a) at least one donor carbohydrate is further contacted with PEP and/or at least one nucleotide.

27. Method according to claim 26, wherein said at least one nucleotide is selected from the group consisting of: CMP, CDP, CTP, UMP, UDP and UTP.

28. Method according to claim 3, wherein said acceptor is oligomeric or polymeric W-135 capsule polysaccharide, oligomeric or polymeric Y capsule polysaccharide, oligomeric or polymeric α2,8-linked sialic acid and/or oligomeric or polymeric α2,9-linked sialic acid; or wherein said acceptor is capsule polysaccharide of Neisseria meningitidis serogroup A or X or a carbohydrate structure containing terminal GlcNAc residues.

29. Method according to claim 28, wherein the acceptor carries one or more additional functional groups at its reducing end.

30. (canceled)

31. Method according to claim 28, wherein the carbohydrate structure containing terminal GlcNAc-residues is selected from the group consisting of: Hyaluronic acid, heparin sulphate, heparan sulphate and protein-linked oligosaccharides.

32. Method according to claim 28, wherein said acceptor capsule polysaccharide is purified and/or hydrolysed.

33. (canceled)

34. A chimeric capsular polysaccharide of Neisseria meningitidis obtainable by the method according to claim 1.

35. A pharmaceutical composition comprising the chimeric capsular polysaccharide of claim 34 and an acceptable pharmaceutical carrier.

36. (canceled)

37. A method for the prophylaxis or treatment of a patient having a Neisseria meningitides disease, the method comprising administering to the patient an effective amount of a chimeric capsular polysaccharide in accordance with claim 34 or a pharmaceutical composition comprising the chimeric capsular polysaccharide and an acceptable pharmaceutical carrier.

38. The method of claim 37, wherein the subject is human.

39. The method of compound of claim 37, wherein the Neisseria meningitides disease is a disease caused by Neisseria meningitidis serogroup A, B, C, W-135, X or Y.

40-41. (canceled)
